(19) United States
(12) Patent Application Publication
(10) Pub. No.: US 2007/0197524 A1
(43) Pub. Date: Aug. 23, 2007

(54) Fluorenes and carbazoles as ligands of the EP2 receptor

(76) Inventors: Nico Brauer, Jena (DE); Bernd Buchmann, Hohen Neuendorf (DE); Christoph Huwe, Berlin (DE); Bernhard Lindenthal, Berlin (DE); Gernot Langer, Berlin (DE); Olaf Peters, Tabarz (DE); Gerd Schubert, Jena (DE); Ulrich Bothe, Berlin (DE); Luisella Toschi, Berlin (DE); Michaele Peters-Kottig, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/642,975

(22) Filed: Dec. 21, 2006

Related U.S. Application Data
(60) Provisional application No. 60/754,186, filed on Dec. 28, 2005.

(30) Foreign Application Priority Data
Dec. 22, 2005 (DE) 10 2005 062 741.2

(51) Int. Cl. A61K 31/5377; A61K 31/4709; A61K 31/428; A61K 31/4178; A61K 31/416; A61K 31/403; C07D 403/02

(52) U.S. Cl. 514/232.5; 514/411; 514/397; 514/367; 514/378; 514/314; 514/406; 544/142; 546/167; 548/444; 548/311.1; 548/159; 548/247; 548/364.1

(57) ABSTRACT

The present invention relates to carbazoles/fluorenes, to processes for their preparation and to their use for preparing pharmaceutical agents for the treatment of disorders connected to the EP₂ receptor.

This application claims the benefit of the filing date of U.S. Provisional Application Ser. No. 60/754,186 filed Dec. 28, 2005.

The present invention relates to carbazoles and fluorenes as EP₂ receptor modulators, to processes for their preparation and to their use as medicaments.

It has long been known that prostaglandins are the key molecules in the processes of female reproductive biology, for example control of ovulation, of fertilization, of nidation, of decidualization (e.g. placenta formation) and of menstruation. Prostaglandins likewise play an important part in the pathological changes in the reproductive tract, including menorrhagia, dysmenorrhea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis and proliferation.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtype to which prostaglandin E₂ binds appears to be of particular interest for the receptor-mediated effects which play a role in the regulation of fertility. Thus, it has been possible to show that reproductive functions are impaired in EP₂-knockout mice (EP₂ ^{-/-}), and that these animals have a smaller “litter size”,(Matsumoto et al., 2001, Biology of Reproduction 64, 1557-1565). It was likewise possible to show that these EP₂-knockout mice (Hizaki et al. Proc Natl Acad Sci U. S. A. 1999 Aug. 31; 96(18):10501-10506) show distinctly reduced cumulus expansion and severe subfertility, demonstrating the significance of the prostaglandin EP₂ receptor for this process. The EP₂ receptor accordingly represents an important target for the development of medicaments for controlling female fertility. The 4 subclasses of the EP₂ receptor open up the possibility of targeted development of selectively active PGE₂ compounds. However, to date, scarcely any selective EP₂ receptor ligands are known, since most of the known compounds also bind to the other EP₂ receptor subtypes such as, for example, to the EP₄ receptor. EP₂ receptor antagonists are described, for example, in the application US 2005059742 (Jabbour, Medical Research Council). What is claimed is a method in which an EP₂ and/or EP₄ antagonist can be used for the treatment of menorrhagia and dysmenorrhea. AH6809 is disclosed as an antagonist of the EP₂ or EP₄ receptor; no other specific antagonists and no new compounds are disclosed.

An early application of the same group (EP1467738) claims EP₂ or EP₄ antagonists for the treatment of pathological states, for example uterus carcinoma, myoma and endometriosis. Likewise, no new compounds are disclosed.

In the application WO 03/016254, Ono Pharmaceutical claims the preparation of benzene acid or saturated carboxylic acid derivatives which have been substituted by aryl or heterocycles, for uses including as prostaglandin E₂ receptor antagonists. The compounds disclosed are claimed for the treatment of a multitude of disorders, including allergic disorders, Alzheimer's disease, pain, abortion, menstrual complaints, menorrhagia and dysmenorrhea, endometriosis, disorders of the bones, ischemia, etc. However, the compounds described feature a particularly high affinity for the EP₃ receptor. A further application (WO 04/032964) describes novel compounds which likewise feature a particularly high affinity for the EP₃ receptor, but also find use as EP₂ antagonists for the treatment and prophylaxis of allergic disorders.

In the application WO 04/39807 of Merck Frosst, Canada, the preparation of pyridopyrrolizines and pyrido-indolizines is disclosed. However, these compounds feature good binding to the PGD₂ receptor; this receptor is another subtype of the prostaglandin receptor.

Napthalene derivatives as EP₄ receptor ligands are disclosed by the SmithKline Beecham Cooperation in the application US 2004102508. The compounds claimed find use for the treatment or prophylaxis of pain, allergic reactions and neurodegenerative disorders.

EP₄ antagonists (γ-lactams) are claimed in the application WO 03/103604 (Applied Research Systems). The compounds bind about 60 times better to the EP₄ than to the EP₂ receptor and are claimed for uses including the treatment of premature labor, dysmenorrhea, asthma, infertility or fertility disorders. In the applications WO 03/053923 (substituted pyrrolidines) or WO 03/035064 (substituted pyrazolidines), the same company claims compounds for the treatment of disorders associated with prostaglandins, for example infertility, hypertension and osteoporosis. The compounds bind to the EP₄ and to the EP₂ receptor. The application WO 03/037433 claims ω-cycloalkyl, 17-heteroaryl prostaglandin derivatives as EP₂ receptor antagonists, especially for the treatment of elevated intraocular pressure.

The application WO 03/064391 (Pfizer Products) describes metabolites of [3-[[N-(4-tert-butylbenzyl) (pyridin-3-ylsulfonyl)amino]methyl]acetic acid which inhibit the binding of [³H] prostaglandin E₂ to the EP₂ receptor. The use of these metabolites for the treatment of osteoporosis is disclosed.

Tani et al. claim, in the application US 2005124577, 8-azaprostaglandin derivatives for the treatment of immunological disorders, allergic disorders, premature labor, abortion, etc. The compounds bind to the EP₂ and to the EP₄ receptor.

European patent EP 1306087 describes EP₂ receptor agonists which are used in the treatment of erectile dysfunction. The same structural class is described in European patent EP 860430, and their use for producing a medicament for the treatment of immunological disorders, asthma and abortion is claimed. The application WO 04/32965 describes the EP₂ receptor agonists which are used for the treatment and prevention of disorders caused by an organ dysfunction caused by ischemia. WO 04/009117 describes EP₂ and EP₄ receptor agonists for the treatment of disorders caused by uterine contraction, for example menstrual complaints.

The applications WO 03/74483 and WO 03/09872 describe agonists which bind equally to the EP₂ and the EP₄ receptor (Ono Pharmaceuticals).

Agonists of the EP₂ and of the EP₄ receptor are frequently described in connection with the treatment of osteoporosis (WO 99/19300, US 2003/0166631, WO 03/77910, WO 03/45371, WO 03/74483 and WO 03/09872) and for glaucoma treatment (WO 04/37813, WO 04/37786, WO 04/19938, WO 03/103772, WO 03/103664, U.S. Pat. Nos. 6,747,037, 6,410,591, WO 03/40123, WO 03/47513, WO 03/47417.

The patent application WO 04/12656 claims EP₂ receptor agonists in connection with inflammation.

The patent application WO 03/77919 claims EP₄ receptor agonists for the treatment of fertility.

Selective EP₂ receptor agonists and antagonists which control the processes which are ultimately responsible for nidation and decidualization and thus for the promotion or inhibition of fertility have not been described to date.

This gives rise to the object of providing stable, selective and effective compounds which bind to the EP₂ receptor for the development of novel medicaments.

It has now been found that, surprisingly, compounds of the general formula D 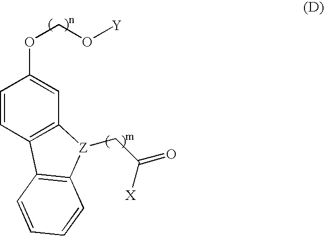
where
Y is a 5-12-membered, mono- or bicyclic aryl or heteroaryl radical which may optionally be mono- or polysubstituted by
   a C₁-C₆-alkyl which may be straight or branched, saturated or unsaturated, and may optionally be mono- or polysubstituted by R¹ where R¹ is the —O—C₁-C₆-alkyl group, cyano, —N(C₁-C₆-alkyl)₂, —NH—C₁-C₆-alkyl, —C₄-C₈-cycloamine, —NH₂, —CO—CH₃—, —C₁-C₆-alkyl, —SO₂—NH₂, —SO₂—NH—CO—C₁-C₆-alkyl, —SO₂—NH—C₁-C₆-alkyl, —SO₂-C₁-C₆-alkyl, —NH—CO—C₁-C₆-alkyl, NO₂, —OH, —COOH or halogen,
   R¹ where R¹ is as defined above,
   a saturated or unsaturated 4-8-membered heterocycle which optionally bears 1-3 nitrogen or oxygen atoms and is optionally substituted by —OCH₃, —COCH₃, —C₁-C₆-alkyl, —C₁-C₂-alkyl-O—CH₃ or a keto group,
   an —SO₂—NH₂, —SO₂—CH₃, —NH—CO—CH₃ group, NO₂, —OH, —COOH or halogen,
   or a fused 5-7-membered carbocycle optionally substituted by a keto group,
   a 4-12-membered, mono- or bicyclic hetero-cycle which may be saturated or unsaturated, is interrupted once or more than once by nitrogen, oxygen or sulfur, and is optionally mono- or polysubstituted by R¹, where R¹ is as defined above, with an oxygen or a keto group,
Z is a carbon or nitrogen radical,
X is an —OH, —NH₂ group, an —O—C₁-C₆-alkyl, —N(C₁-C₆-alkyl)₂, —NH—C₁-C₆-alkyl, C₄-C₈-cyclo-amine, —NH—SO₂—C₁-C₆-alkyl or a saturated or unsaturated —C₃-C₈-cycloalkyl radical which may optionally be substituted,
n is 1-7,
m is 1-4 and their salts with physiologically accept-able bases and their cyclodextrin clathrates overcome the known disadvantages, and a better selectivity for the EP₂ receptor and hence better activity and prolonged action can be achieved.

The saturated, unbranched C₁-C₆-alkyl substituents specified under Y are, for example, a methyl, ethyl, n-propyl, n-butyl, n-pentyl, n-hexyl group; the branched C₃-C₆-alkyl groups are an isopropyl, isobutyl, sec-butyl, tert-butyl, isopentyl, neopentyl, 2-methylpentyl, 2,2-dimethylbutyl, 2,3-dimethylbutyl group.

The alkyl groups may optionally be mono- to polysubstituted by halogen atoms, for example fluorine, chlorine or bromine.

Unsaturated C₁-C₆-alkyl is understood to mean the following:
vinyl, allyl, homoallyl, (E)-but-2-enyl, (Z)-but-2-enyl, pent-4-enyl, (E)-pent-3-enyl, (Z)-pent-3-enyl, (E)-pent-2-enyl, (Z)-pent-2-enyl, 2-methylvinyl, 3-methylbut-3-enyl, 2-methylbut-3-enyl, (E)-2-methylbut-2-enyl, (Z)-2-methylbut-2-enyl, 2-ethylprop-2-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, 1-methylpent-4-enyl, (E)-1-methylpent-3-enyl, (Z)-1-methylpent-3-enyl, 1-ethylbut-3-enyl, (E)-1-methylpent-2-enyl, (Z)-1-methylpent-2-enyl.

Halogen is understood to mean the following: fluorine, chlorine, bromine, iodine.

C₁-C₆-Cycloalkyl is understood to mean monocyclic alkyl rings such as cyclopropyl, cyclobutyl, cyclopentyl, cyclohexyl or cycloheptyl, or cyclooctyl, but also bicyclic rings, for example decahydronaphthalene, tricyclic rings, or bridged rings, for example adamantanyl.

An —O—C₁-C₆-alkyl radical is in each case understood to mean a straight-chain or branched C₁-C₆-alkoxy radical, for example methoxy, ethoxy, propoxy, isopropoxy, butoxy, isobutoxy, sec-butoxy, pentoxy, isopentoxy, hexoxy.

A 5-12-membered, mono- or bicyclic aryl or heteroaryl radical which may optionally be mono- or polysubstituted is understood to mean 5-12-membered ring systems which, in place of the carbon, may contain one or more identical or different heteroatoms, such as oxygen, nitrogen or sulfur, in the ring, may be mono-, bi- or tricyclic and may additionally in each case be benzofused.

Examples of a 5-12-membered, mono- or bicyclic aryl radical include the following: cyclopentadienyl, phenyl, tropyl, cyclooctadienyl, indenyl, naphthyl, azulenyl, biphenyl.

The 5-12-membered, mono- or bicyclic heteroaryl groups may be a pyridinyl, pyrimidinyl, quinolinyl, isoquinolinyl, benzofuranyl, benzothienyl, 1,3-benzodioxolyl, 2,1,3-benzothiadiazolyl, indolyl, furanyl, thienyl, oxazolyl, isoxazolyl, thiazolyl, pyrrolyl, pyrazolyl, pyrazinyl, pyridazinyl or imidazolyl group bonded via one of the substitutable positions.

The 4-12-membered, saturated or unsaturated, mono- or bicyclic heterocycles may be a piperidinyl, morpholinyl, thiomorpholinyl, piperazinyl, tetrahydro-furanyl, tetrahydrothienyl, imidazolidinyl or pyrrolidinyl group bonded via one of the substitutable positions.

The 4-8-membered, saturated or unsaturated heterocycle which is possible as a substituent of the (Het)aryl radical in Y may be the following radical: furanyl, thiophenyl, pyrazoyl, pyrrolyl, oxazolyl, thiazolyl, imidazolyl, where the heterocycle may bear methyl, ethyl or propyl groups at any one or more than one position, and may be bonded to the parent system via any carbon atom.

A fused 5-7-membered carbocycle is understood to mean, for example, a cyclopentyl, cyclohexyl or cycloheptyl radical which is optionally substituted by a keto group; examples include: cyclopentanone, cyclohexanone or cycloheptanone.

A C₄-C₈ cycloamine radical is understood to mean 4- to 8-membered ring systems, for example piperidine, morpholine, piperazine, azetidine, azepane, diazepane. The ring system may optionally be interrupted by sulfur, oxygen or nitrogen, where the nitrogen may be substituted by R¹, where R¹ is as defined above.

When an acidic function is present, suitable salts are the physiologically acceptable salts of organic and inorganic bases, for example the readily soluble alkali metal and alkaline earth metal salts, and also N-methylglucamine, dimethylglucamine, ethylglucamine, lysine, 1,6-hexadiamine, ethanolamine, glucosamine, sarcosine, serinol, tris(hydroxymethyl)aminomethane, aminopropanediol, Sovak base, 1-amino-2,3,4-butane-triol.

The hydroxyl group in X may be protected by one of the customary hydroxyl protecting groups known to those skilled in the art.

The customary hydroxyl protecting groups are described comprehensively in T. W. Greene, P. G. M. Wuts “Protective Groups in Organic Synthesis”, 2nd Edition, John Wiley & Sons, 1991).

The protecting groups are preferably alkyl-, aryl- or mixed alkylaryl-substituted silyl groups, for example the trimethylsilyl (TMS), triethylsilyl (TES), tert-butyldimethylsilyl (TBDMS), tert-butyldiphenylsilyl (TBDPS) or triisopropylsilyl groups (TIPS), or another common hydroxyl protecting group (methoxymethyl, methoxyethoxymethyl, ethoxyethyl, tetrahydrofuranyl and tetrahydropyranyl groups).

The free C₁-C₆-alkyl alcohols or C₁-C₆-alkylcarboxylic acids in X may also be present in the form of esters and are thus prodrugs of the physiological compounds of the general formula D, which metabolize in the organism to give compounds of the general formula D.

Suitable compounds are detailed, for example, in Hans Bundgaard (editor), Design of Prodrugs, Elsevier, Amsterdam 1985.

C1-C₆-Alkyl alcohols are understood to mean straight or branched alcohols which may, for example, also be protected as the acetate, propionate, butyrate, valerate and caproate.

The esters of the C₁-C₆-alkylcarboxylic acids may be straight or branched C₁-C₆-alkyl esters; examples include: methyl esters, acetyl esters, propyl esters, isopropyl esters, butyl esters, pentyl esters, hexyl esters.

Preference is given to the compounds of the general formula D where
Y is a 5-12-membered, mono- or bicyclic aryl or heteroaryl radical which may optionally be mono- or polysubstituted by
   a C₁-C₆-alkyl which may be straight or branched, saturated or unsaturated, and may optionally be mono- or poly-substituted by R², where R² is the —O—C₁-C₃-alkyl group, cyano, —N(C₁-C₃-alkyl)₂, NH—C₁-C₃-alkyl, —NH₂, —CO—CH₃—, —C₁-C₃-alkyl, —SO₂—NH₂, —SO₂—NH—CO—C₁-C₃-alkyl, SO₂—NHC₁-C₃-alkyl, —SO₂—C₁-C₃-alkyl, —NH—CO—C₁-C₃-alkyl, NO₂, —OH, —COOH, —C₄-C₆-cycloamine or halogen,
   R², where R² is as defined above,
   a saturated or unsaturated 5-7-membered heterocycle which optionally bears 1-3 nitrogen or oxygen atoms and is optionally substituted by —OCH₃, —C₁-C₃-alkyl, —C₁-C₂-alkyl-OCH₃ or a keto group,
   an —SO₂—NH₂, —SO₂—CH₃, —NH—CO—CH₃ group, NO₂, —OH, —COOH or halogen,
   or by a fused 5-7-membered carbocycle optionally substituted by a keto group,
   a 6-12-membered, mono- or bicyclic hetero-cycle which may be saturated or unsaturated, is interrupted once or more than once by nitrogen, oxygen or sulfur, and is optionally mono- or polysubstituted by an —OCH₃, —CO—CH₃ group, —C₁-C₃-alkyl, cyano, oxygen or a keto group,
z is a carbon or nitrogen radical,
X is an OH or NH₂ group, an —O—C₁-C₆-alkyl, —N(C₁-C₆-alkyl)₂, —NH—C₁-C₆-alkyl, —C₄-C₈-cycloamine, —NH—SO₂—C₁-C₆-alkyl or a saturated or unsaturated —C₃-C₈-cycloalkyl radical which may optionally be substituted,
n is 2-5,
m is 1-3.

Preference is likewise given to the compounds of the general formula D where
Y is a 6-12-membered mono- or bicyclic aryl radical which may optionally be mono- or polysubstituted and which is optionally mono- or polysubstituted by
   a C₁-C₆-alkyl which may be straight or branched, saturated or unsaturated, and may optionally be mono- or poly-substituted by R³, where R³ is the —OCH₃ group, —CO—CH₃, cyano, —NH₂, —N (CH₃)₂, —NHCH₃, —SO₂—NH₂, —SO₂—NH—CO—CH₃, —SO₂—NH—CH₃, —SO₂—CH₃, —NH—CO—CH₃, —NO₂, —OH, —COOH or halogen,
R³, where R³ is as defined above,
   an —SO₂—NH₂, —SO₂—CH₃, —NH—CO—CH₃ group, NO₂, —OH, —COOH or halogen,
   or by the group 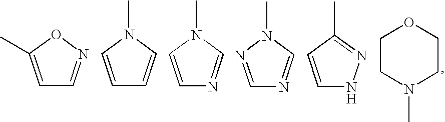
   represent the group 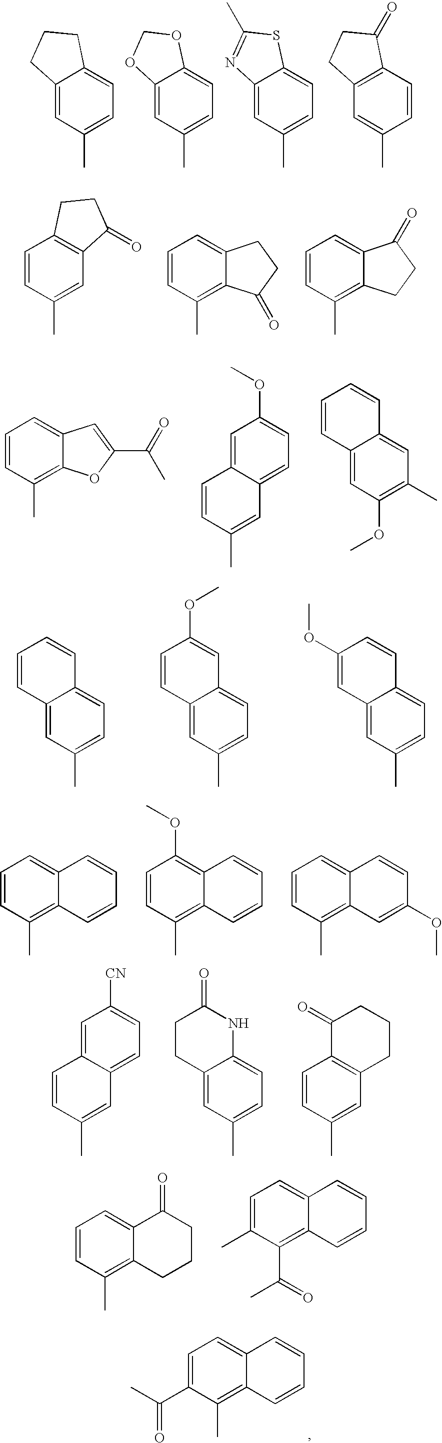
Z is a carbon or a nitrogen radical,
X is a hydroxyl group, —N(CH₃)₂, —NH—CH₃, —NHSO₂—CH₃,
n is 3-5,
m is 1-2.

Particular preference is given to the following compounds:
(R/S) [2-(4-m-tolyloxybutoxy)-9H-fluoren-9-yl]acetic acid
(R/S) {2-[4-(3-hydroxy-5-methoxyphenoxy)butoxy)]-9H-fluoren-9-yl}acetic acid
(R/S) {2-[4-(3-hydroxyphenoxy)butoxy]-9H-fluoren-9-yl}acetic acid
(R/S) {2-[4-(3-acetylaminophenoxy)butoxy]-9H-fluoren-9-yl}acetic acid
ethyl {2-[4-(3-hydroxyphenoxy)butoxy]carbazol-9-yl}-acetate
ethyl [2-(4-m-tolyloxybutoxy)carbazol-9-yl]acetate
ethyl {2-[4-(3-acetylaminophenoxy)butoxy)carbazol-9-yl}acetate
ethyl {2-[4-(3-hydroxy-5-methoxyphenoxy)butoxy]-carbazol-9-yl}acetate
methyl 3-[4-(9-ethoxycarbonylmethyl-9H-carbazol-2-yloxy)butoxy]benzoate
ethyl 3-{2-[4-(3-hydroxyphenoxy)butoxy]carbazol-9-yl}propionate
ethyl 3-[2-(4-m-tolyloxybutoxy)carbazol-9-yl]propionate
ethyl 3-{2-[4-(3-acetylaminophenoxy)butoxy]carbazol-9-yl}propionate
ethyl 3-{2-[4-(3-hydroxy-5-methoxyphenoxy)butoxy]-carbazol-9-yl}propionate
methyl 3-{4-[9-(2-ethoxycarbonylethyl)-9H-carbazol-2-yloxy]butoxy}benzoate
{2-[4-(3-hydroxyphenoxy)butoxy]carbazol-9-yl}acetic acid
[2-(4-m-tolyloxybutoxy)carbazol-9-yl]acetic acid
{2-[4-(3-acetylaminophenoxy)butoxy]carbazol-9-yl}acetic acid
{2-[4-(3-hydroxy-5-methoxyphenoxy)butoxy]carbazol-9-yl}acetic acid
3-[4-(9-carboxymethyl-9H-carbazol-2-yloxy)butoxy]-benzoic acid
3-{2-[4-(3-hydroxyphenoxy)butoxy]carbazol-9-yl}propionic acid
3-[2-(4-m-tolyloxybutoxy)carbazol-9-yl]propionic acid
3-{2-[4-(3-acetylaminophenoxy)butoxy]carbazol-9-yl}propionic acid
3-{2-[4-(3-hydroxy-5-methoxyphenoxy)butoxy]carbazol-9-yl}propionic acid
3-{4-[9-(2-carboxyethyl)-9H-carbazol-2-yloxy]butoxy}-benzoic acid
{2-[4-(2-acetylphenoxy)butoxy]carbazol-9-yl}acetic acid
{2-[4-(3-acetylphenoxy)butoxy]carbazol-9-yl}acetic acid
{2-[4-(4-nitrophenoxy)butoxy]carbazol-9-yl}acetic acid
{2-[4-(3-nitrophenoxy)butoxy]carbazol-9-yl}acetic acid
{2-[4-(3-oxoindan-4-yloxy)butoxy]carbazol-9-yl}acetic acid
{2-[4-(4-chlorophenoxy)butoxy]carbazol-9-yl}acetic acid
{2-[4-(3-fluorophenoxy)butoxy]carbazol-9-yl}acetic acid
{2-[4-(3,5-dimethoxyphenoxy)butoxy]carbazol-9-yl}acetic acid
{2-[4-(2-fluorophenoxy)butoxy]carbazol-9-yl}acetic acid
{2-[4-(2-pyrrol-1-ylphenoxy)butoxy]carbazol-9-yl}acetic acid
{2-(4-(3-isopropylphenoxy)butoxy]carbazol-9-yl}acetic acid
{2-[4-(4-pyrrol-1-ylphenoxy)butoxy]carbazol-9-yl}acetic acid
{2-[4-(2-isoxazol-5-ylphenoxy)butoxy]carbazol-9-yl}acetic acid
{2-[4-(2-isoxazol-5-yl-4-methylphenoxy)butoxy]-carbazol-9-yl}acetic acid
{2-[4-(2-acetylbenzofuran-7-yloxy)butoxy]carbazol-9-yl}acetic acid
{2-[4-(2-morpholin-4-ylphenoxy)butoxy]carbazol-9-yl}acetic acid
{2-[4-(6-methoxynaphthalen-2-yloxy)butoxy]carbazol-9-yl}acetic acid
{2-[4-(2-chlorophenoxy)butoxy]carbazol-9-yl}acetic acid
{2-[4-(3-chlorophenoxy)butoxy]carbazol-9-yl}acetic acid
{2-[4-(4-fluorophenoxy)butoxy]carbazol-9-yl}acetic acid
{2-[4-(4-[1,2,4]triazol-1-ylphenoxy]butoxy}carbazol-9-yl)acetic acid
(2-{4-[2-(1H-pyrazol-3-yl)phenoxy]butoxy}carbazol-9-yl)acetic acid
3-[2-(4-phenoxybutoxy)carbazol-9-yl]propionic acid
3-{2-[4-(2-acetylphenoxy)butoxy]carbazol-9-yl}propionic acid
3-{2-[4-(3-acetylphenoxy)butoxy]carbazol-9-yl}propionic acid
3-{2-[4-(4-acetylphenoxy)butoxy]carbazol-9-yl}propionic acid
3-{2-[4-(4-acetylaminophenoxy)butoxylcarbazol-9-yl}propionic acid
3-{2-[4-(4-methoxyphenoxy)butoxy]carbazol-9-yl}propionic acid
3-{2-[4-(4-nitrophenoxy)butoxy]carbazol-9-yl}propionic acid
3-{2-[4-(2-nitrophenoxy)butoxy]carbazol-9-yl}propionic acid
3-{2-[4-(3-oxoindan-4-yloxy)butoxy]carbazol-9-yl}propionic acid
3-{2-[4-(5-oxo-5,6,7,8-tetrahydronaphthalen-2-yloxy)butoxy]carbazol-9-yl}propionic acid
3-{2-[4-(1-oxoindan-5-yloxy)butoxy]carbazol-9-yl}propionic acid
3-{2-[4-(4-chlorophenoxy)butoxy]carbazol-9-yl}propionic acid
3-{2-[4-(2-methoxyphenoxy)butoxy]carbazol-9-yl}propionic acid
3-[2-(4-o-tolyloxybutoxy)carbazol-9-yl]propionic acid
3-[2-(4-p-tolyloxybutoxy)carbazol-9-yl]propionic acid
3-{2-[4-(3-methoxyphenoxy)butoxy]carbazol-9-yl}propionic acid
3-{2-[4-(naphthalen-2-yloxy)butoxy]carbazol-9-yl}propionic acid
3-{2-[4-(1-oxoindan-4-yloxy)butoxy]carbazol-9-yl}propionic acid
3-{2-[4-(5-oxo-5,6,7,8-tetrahydronaphthalen-1-yloxy)butoxy]carbazol-9-yl}propionic acid
3-{2-[4-(3-fluorophenoxy)butoxy]carbazol-9-yl}propionic acid
3-{2-[4-(3,5-dimethoxyphenoxy)butoxy]carbazol-9-yl}propionic acid
3-{2-(4-(4-imidazol-1-ylphenoxy)butoxy]carbazol-9-yl}propionic acid
3-{2-[4-(3-oxoindan-5-yloxy)butoxy]carbazol-9-yl}propionic acid
3-{2-[4-(3,4-dimethoxyphenoxy)butoxy]carbazol-9-yl}propionic acid
3-{2-[4-(2-fluorophenoxy)butoxy]carbazol-9-yl}propionic acid
3-{2-[4-(2-pyrrol-1-ylphenoxy)butoxy]carbazol-9-yl}propionic acid
3-{2-[4-(benzo[1,3]dioxol-5-yloxy)butoxy]carbazol-9-yl}propionic acid
3-(2-{4-[4-(2-oxopropyl)phenoxy]butoxy}carbazol-9-yl)propionic acid
3-{2-[4-(3-dimethylaminophenoxy)butoxy]carbazol-9-yl}propionic acid
3-{2-[4-(4-methoxymethylphenoxy)butoxy]carbazol-9-yl}propionic acid
3-{2-[4-(4-dimethylaminomethylphenoxy)butoxy]-carbazol-9-yl}propionic acid
3-(2-{4-[4-(2-methoxyethyl)phenoxy]butoxylcarbazol-9-yl)propionic acid
3-{2-[4-(2,3-dimethoxyphenoxy)butoxy]carbazol-9-yl}propionic acid
3-{2-[4-(2-isoxazol-5-ylphenoxy)butoxy]carbazol-9-yl}propionic acid
3-{2-[4-(2-oxo-1,2,3,4-tetrahydroquinolin-6-yloxy)butoxy]carbazol-9-yl}propionic acid
3-{2-[4-(2-methylbenzothiazol-5-yloxy)butoxy]-carbazol-9-yl}propionic acid
3-{2-[4-(2-methanesulfonylphenoxy)butoxy]carbazol-9-yl}propionic acid
3-{2-[4-(4-sulfamoylphenoxy)butoxy]carbazol-9-yl}propionic acid
3-{2-[4-(3-methoxynaphthalen-2-yloxy)butoxy]carbazol-9-yl}propionic acid
3-{2-[4-(2-isoxazol-5-yl-4-methylphenoxy)butoxy]-carbazol-9-yl}propionic acid
3-{2-[4-(2-acetylbenzofuran-7-yloxy)butoxy]carbazol-9-yl}propionic acid
3-{2-[4-(1-acetylnaphthalen-2-yloxy)butoxy]carbazol-9-yl}propionic acid
3-{2-[4-(3-morpholin-4-ylphenoxy)butoxy]carbazol-9-yl}propionic acid
3-{2-[4-(6-methoxynaphthalen-2-yloxy)butoxy]carbazol-9-yl}propionic acid
3-{2-[4-(4-cyanomethylphenoxy)butoxy]carbazol-9-yl}propionic acid
3-{2-[4-(4-cyanophenoxy)butoxy]carbazol-9-yl}propionic acid
3-{2-[4-(2-cyanophenoxy)butoxy]carbazol-9-yl}propionic acid
3-{2-[4-(2-chlorophenoxy)butoxy]carbazol-9-yl}propionic acid
3-{2-[4-(3-chlorophenoxy)butoxy]carbazol-9-yl}propionic acid
3-{2-[4-(4-fluorophenoxy)butoxy]carbazol-9-yl}propionic acid
3-{2-[4-(4-[1,2,4]triazol-1-ylphenoxy)butoxy]-carbazol-9-yl}propionic acid
3-(2-{4-[2-(1H-pyrazol-3-yl)phenoxy]butoxy}carbazol-9-yl)propionic acid
[2-(3-phenoxypropoxy)carbazol-9-yl]acetic acid
{2-[3-(2-acetylphenoxy)propoxy]carbazol-9-yl}acetic acid
{2-[3-(3-acetylphenoxy)propoxy]carbazol-9-yl}acetic acid
{2-[3-(4-acetylphenoxy)propoxy]carbazol-9-yl}acetic acid
{2-[3-(4-acetylaminophenoxy)propoxy]carbazol-9-yl}acetic acid
{2-[3-(4-methoxyphenoxy)propoxy]carbazol-9-yl}acetic acid
{2-[3-(3-nitrophenoxy)propoxy]carbazol-9-yl}acetic acid
{2-[3-(2-nitrophenoxy)propoxy]carbazol-9-yl}acetic acid
{2-[3-(3-oxoindan-4-yloxy)propoxy]carbazol-9-yl}acetic acid
{2-[3-(5-oxo-5,6,7,8-tetrahydronaphthalen-2-yloxy)propoxy]carbazol-9-yl}acetic acid
{2-[3-(1-oxoindan-5-yloxy)propoxy]carbazol-9-yl}acetic acid
{2-[3-(4-chlorophenoxy)propoxy]carbazol-9-yl}acetic acid
[2-(3-o-tolyloxypropoxy)carbazol-9-yl]acetic acid
[2-(3-m-tolyloxypropoxy)carbazol-9-yl]acetic acid
[2-(3-p-tolyloxypropoxy)carbazol-9-yl]acetic acid
{2-[3-(3-methoxyphenoxy)propoxy]carbazol-9-yl}acetic acid
(2-[3-(naphthalen-1-yloxy)propoxy]carbazol-9-yl}acetic acid
{2-[3-(3-acetylaminophenoxy)propoxy)carbazol-9-yl}acetic acid
{2-[3-(1-oxoindan-4-yloxy)propoxy]carbazol-9-yl}acetic acid
{2-[3-(5-oxo-5,6,7,8-tetrahydronaphthalen-1-yloxy)propoxy]carbazol-9-yl}acetic acid
{2-[3-(3-fluorophenoxy)propoxy]carbazol-9-yl}acetic acid
{2-[3-(3,5-dimethoxyphenoxy)propoxy]carbazol-9-yl}acetic acid
{2-(3-(4-imidazol-1-ylphenoxy)propoxy]carbazol-9-yl}acetic acid
{2-[3-(3-oxoindan-5-yloxy)propoxy]carbazol-9-yl}acetic acid
{2-[3-(3,4-dimethoxyphenoxy)propoxy]carbazol-9-yl}acetic acid
{2-[3-(2-acetylaminophenoxy)propoxy]carbazol-9-yl}acetic acid
{2-[3-(7-methoxynaphthalen-2-yloxy)propoxy]carbazol-9-yl}acetic acid
{2-[3-(2-fluorophenoxy)propoxy]carbazol-9-yl}acetic acid
{2-[3-(2-pyrrol-1-ylphenoxy)propoxy]carbazol-9-yl}acetic acid
{2-[3-(benzo[1,3]dioxol-5-yloxy)propoxy]carbazol-9-yl}acetic acid
(2-{3-[4-(2-oxopropyl)phenoxy]propoxy}carbazol-9-yl)acetic acid
{2-[3-(4-methoxynaphthalen-1-yloxy)propoxy]carbazol-9-yl}acetic acid
{2-[3-(2-isopropylphenoxy)propoxy]carbazol-9-yl}acetic acid
{2-[3-(3-isopropylphenoxy)propoxy]carbazol-9-yl}acetic acid
{2-[3-(3-dimethylaminophenoxy)propoxy]carbazol-9-yl}acetic acid
{2-[3-(2-dimethylaminomethylphenoxy)propoxy]carbazol-9-yl}acetic acid
(2-{3-[4-(2-methoxyethyl)phenoxy]propoxy}carbazol-9-yl)acetic acid
{2-[3-(2,3-dimethoxyphenoxy)propoxy]carbazol-9-yl}acetic acid
{2-[3-(2-isoxazol-5-ylphenoxy)propoxy]carbazol-9-yl}acetic acid
{2-[3-(2-methylbenzothiazol-5-yloxy)propoxy]carbazol-9-yl}acetic acid
{2-[3-(2-methanesulfonylphenoxy)propoxy]carbazol-9-yl}acetic acid
{2-[3-(3-methoxynaphthalen-2-yloxy)propoxy]carbazol-9-yl}acetic acid
{2-[3-(7-methoxynaphthalen-1-yloxy)propoxy]carbazol-9-yl}acetic acid
{2-[3-(2-isoxazol-5-yl-4-methylphenoxy)propoxy]-carbazol-9-yl}acetic acid
{2-[3-(2-acetylbenzofuran-7-yloxy)propoxy]carbazol-9-yl}acetic acid
{2-[3-(2-morpholin-4-ylphenoxy)propoxy]carbazol-9-yl}acetic acid
{2-[3-(2-acetylnaphthalen-1-yloxy)propoxy]carbazol-9-yl}acetic acid
{2-[3-(1-acetylnaphthalen-2-yloxy)propoxy]carbazol-9-yl}acetic acid
{2-[3-(3-morpholin-4-ylphenoxy)propoxy]carbazol-9-yl}acetic acid
{2-[3-(4-methanesulfonylphenoxy)propoxy]carbazol-9-yl}acetic acid
{2-[3-(2-cyanophenoxy)propoxy]carbazol-9-yl}acetic acid
{2-[3-(2-chlorophenoxy)propoxy]carbazol-9-yl}acetic acid
{2-[3-(3-chlorophenoxy)propoxy]carbazol-9-yl}acetic acid
{2-[3-(4-fluorophenoxy)propoxy]carbazol-9-yl}acetic acid
(2-{3-[2-(1H-pyrazol-3-yl)phenoxy]propoxy}carbazol-9-yl)acetic acid
[2-(5-phenoxypentyloxy)carbazol-9-yl]acetic acid
{2-[5-(2-acetylphenoxy)pentyloxy]carbazol-9-yl}acetic acid
{2-[5-(3-acetylphenoxy)pentyloxy]carbazol-9-yl}acetic acid
{2-[5-(4-acetylphenoxy)pentyloxy]carbazol-9-yl}acetic acid
{2-[5-(4-acetylaminophenoxy)pentyloxy]carbazol-9-yl}acetic acid
{2-[5-(4-methoxyphenoxy)pentyloxy]carbazol-9-yl}acetic acid
{2-[5-(3-cyanophenoxy)pentyloxy]carbazol-9-yl}acetic acid
{2-[5-(4-nitrophenoxy)pentyloxy]carbazol-9-yl}acetic acid
{2-[5-(3-nitrophenoxy)pentyloxy]carbazol-9-yl}acetic acid
{2-[5-(2-nitrophenoxy)pentyloxy]carbazol-9-yl}acetic acid
{2-[5-(3-oxoindan-4-yloxy)pentyloxy]carbazol-9-yl}acetic acid
{2-[5-(5-oxo-5,6,7,8-tetrahydronaphthalen-2-yloxy)pentyloxy]carbazol-9-yl}acetic acid
{2-[5-(1-oxoindan-5-yloxy)pentyloxy]carbazol-9-yl}acetic acid
{2-[5-(4-chlorophenoxy)pentyloxy]carbazol-9-yl}acetic acid
{2-[5-(2-methoxyphenoxy)pentyloxy]carbazol-9-yl}acetic acid
[2-(5-o-tolyloxypentyloxy)carbazol-9-yl]acetic acid
[2-(5-m-tolyloxypentyloxy)carbazol-9-yl]acetic acid
[2-(5-p-tolyloxypentyloxy)carbazol-9-yl]acetic acid
{2-[5-(3-methoxyphenoxy)pentyloxy]carbazol-9-yl}acetic acid
{2-[5-(naphthalen-1-yloxy)pentyloxy]carbazol-9-yl}acetic acid
{2-[5-(3-acetylaminophenoxy)pentyloxy]carbazol-9-yl}acetic acid
{2-[5-(1-oxoindan-4-yloxy)pentyloxy]carbazol-9-yl}acetic acid
{2-[5-(5-oxo-5,6,7,8-tetrahydronaphthalen-1-yloxy)pentyloxy]carbazol-9-yl}acetic acid
{2-[5-(3-fluorophenoxy)pentyloxy]carbazol-9-yl}acetic acid
{2-[5-(3,5-dimethoxyphenoxy)pentyloxy]carbazol-9-yl}acetic acid
{2-[5-(4-imidazol-1-ylphenoxy)pentyloxy]carbazol-9-yl}acetic acid
{2-[5-(3-oxoindan-5-yloxy)pentyloxy]carbazol-9-yl}acetic acid
{2-[5-(3,4-dimethoxyphenoxy)pentyloxy]carbazol-9-yl}acetic acid
{2-[5-(2-acetylaminophenoxy)pentyloxy]carbazol-9-yl}acetic acid
{2-[5-(2-fluorophenoxy)pentyloxy]carbazol-9-yl}acetic acid
{2-[5-(2-pyrrol-1-ylphenoxy)pentyloxy]carbazol-9-yl}acetic acid
{2-[5-(benzo[1,3]dioxol-5-yloxy)pentyloxy]carbazol-9-yl}acetic acid
{2-[5-(4-isopropylphenoxy)pentyloxy]carbazol-9-yl}acetic acid
{2-[5-(indan-5-yloxy)pentyloxy]carbazol-9-yl}acetic acid
{2-[5-(4-methoxynaphthalen-1-yloxy)pentyloxy]-carbazol-9-yl}acetic acid
{2-[5-(2-isopropylphenoxy)pentyloxy]carbazol-9-yl}acetic acid
{2-[5-(3-isopropylphenoxy)pentyloxy]carbazol-9-yl}acetic acid
{2-[5-(3-dimethylaminophenoxy)pentyloxy]carbazol-9-yl}acetic acid
{2-[5-(2-dimethylaminomethylphenoxy)pentyloxy]-carbazol-9-yl}acetic acid
(2-{5-[4-(2-methoxyethyl)phenoxy]pentyloxy}carbazol-9-yl)acetic acid
{2-[5-(2,3-dimethoxyphenoxy)pentyloxy]carbazol-9-yl}acetic acid
{2-[5-(2-isoxazol-5-ylphenoxy)pentyloxy]carbazol-9-yl}acetic acid
{2-[5-(2-oxo-1,2,3,4-tetrahydroquinolin-6-yloxy)pentyloxy]carbazol-9-yl}acetic acid
{2-[5-(6-cyanonaphthalen-2-yloxy)pentyloxy]carbazol-9-yl}acetic acid
{2-[5-(2-methanesulfonylphenoxy)pentyloxy]carbazol-9-yl}acetic acid
{2-[5-(2-isoxazol-5-yl-4-methylphenoxy)pentyloxy]-carbazol-9-yl}acetic acid
{2-[5-(2-morpholin-4-ylphenoxy)pentyloxy]carbazol-9-yl}acetic acid
{2-[5-(2-acetylnaphthalen-1-yloxy)pentyloxy]carbazol-9-yl}acetic acid
{2-[5-(3-morpholin-4-ylphenoxy)pentyloxy]carbazol-9-yl}acetic acid
{2-[5-(6-methoxynaphthalen-2-yloxy)pentyloxy]-carbazol-9-yl}acetic acid
{2-[5-(4-cyanomethylphenoxy)pentyloxy]carbazol-9-yl}acetic acid
{2-[5-(4-methanesulfonylphenoxy)pentyloxy]carbazol-9-yl}acetic acid
{2-[5-(4-cyanophenoxy)pentyloxy]carbazol-9-yl}acetic acid
{2-[5-(2-cyanophenoxy)pentyloxy]carbazol-9-yl}acetic acid
{2-[5-(2-chlorophenoxy)pentyloxy]carbazol-9-yl}acetic acid
{2-[5-(4-fluorophenoxy)pentyloxy]carbazol-9-yl}acetic acid
{2-[5-(4-[1,2,4]triazol-1-ylphenoxy)pentyloxy]-25 carbazol-9-yl}acetic acid
(2-{5-[2-(1H-pyrazol-3-yl)phenoxy]pentyloxy}carbazol-9-yl)acetic acid
3-[2-(4-m-tolyloxybutoxy)carbazol-9-yl]propionic acid
3-{2-[4-(3-acetylaminophenoxy)butoxy]carbazol-9-30 yl}propionic acid
N-ethyl-2-[2-(4-m-tolyloxybutoxy)carbazol-9-yl]acetamide
N,N-diethyl-2-[2-(4-m-tolyloxybutoxy)carbazol-9-yl]acetamide
N-{2-[2-(4-m-tolyloxybutoxy)carbazol-9-yl]acetyl}-methanesulfonamide.

Compared with the known prostaglandin E₂ ligands, the novel EP₂ agonists and antagonists are notable for greater selectivity and stability.

The present invention provides medicaments for the treatment and prophylaxis of disorders which include fertility disorders, infectious diseases, cancer, viral infections, cardiovascular disorders, elevated intraocular pressure, glaucoma, disorders of the skeletal system, angiogenic disorders, abnormalities of uterine contraction, pain, neuroinflammatory disorders, immunomodulatory infections and nephrological disorders.

Fertility disorders are understood to mean disorders leading to no ovulation taking place, to nidation of a fertilized oocyte not taking place and no decidualisation taking place; infectious diseases are understood to mean diseases caused by unicellular parasites; cancer is understood to mean solid tumors and leukemia; viral infections are understood to mean, for example, cytomegalus infections, hepatitis, hepatitis B and C and HIV disorders; immunomodulatory infections are understood to mean, for example, bird flu; cardiovascular disorders are understood to mean ischemic reperfusion disorder, stenoses, arterioscleroses and restenoses; angiogenic disorders are understood to mean, for example, endometriosis and fibrosis; elevated intraocular pressure is understood to mean glaucoma; abnormalities of uterine contraction are understood to mean, for example, menstrual complaints; disorders of the skeletal system are understood to mean osteoporosis; neuroinflammatory disorders are understood to mean multiple sclerosis, Alzheimer's disease, pain; and nephrological disorders are understood to mean glomerulonephritis.

The present invention likewise provides medicaments for the treatment and prophylaxis of the disorders listed above, which comprise at least one compound of the general formula D, and also medicaments comprising suitable formulation and carrier substances.

For use of the compounds according to the invention as medicaments, they are converted to the form of a pharmaceutical product which, in addition to the active ingredient, comprises pharmaceutical, organic or inorganic inert carrier materials suitable for enteral or parenteral administration, for example water, gelatin, gum arabic, lactate, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositories, emulsions, or in liquid form, for example as solutions, suspensions or emulsions.

If appropriate, they comprise excipients intended to function, for example, as fillers, binders, disintegrants, lubricants, solvents, solubilizers, masking flavors, dye, emulsifiers. Excipient types in the context of the invention are, for example, saccharides (mono-, di-, tri-, oligo- and/or polysaccharides), fats, waxes, oils, hydrocarbons, anionic, nonionic, cationic natural, synthetic or semisynthetic surfactants. If appropriate, they additionally comprise excipients such as preservatives, stabilizers, wetting agents or emulsifiers; salts to alter the osmotic pressure or buffers.

The present invention likewise provides these pharmaceutical products.

Aerosol solutions are appropriately produced for inhalation.

Particularly suitable for oral use are tablets, coated tablets or capsules with talc and/or carbohydrate carriers or binders, for example lactose, maize starch or potato starch. Use is also possible in liquid form, for example as fluid to which a sweetener is added where appropriate.

Sterile, injectable, aqueous or oily solutions are used for parenteral administration. Solutions for injection or suspensions are particularly suitable; especially aqueous solutions of the active compounds in polyethoxylated castor oil are suitable.

Suppositories, tampons or intrauterine devices, for example, are suitable and customary for vaginal administration.

For intraarticular injection, it is possible to use appropriately formulated crystal suspensions.

For intramuscular injection, it is possible to use aqueous and oily injection solutions or suspensions and corresponding depot preparations.

For rectal administration, it is possible to use the novel compounds in the form of suppositories, capsules, solutions (for example in the form of enemas) and ointments both for systemic and for local therapy.

For pulmonary administration of the novel compounds, they can be used in the form of aerosols and inhalations.

For local administration on eyes, the external auditory canal, middle ear, nasal cavity and paranasal sinuses, the novel compounds may be used as drops, ointments and tinctures in appropriate pharmaceutical formulations.

For topical administration, formulations in gels, ointments, greasy ointments, creams, pastes, powder, milk and tinctures are possible. The dosage of the compounds of the general formula D in these formulations should be 0.01%-20% in order to achieve a sufficient pharmacological effect.

Carrier systems which can also be used are surface-active excipients such as salts of bile acids or animal or vegetable phospholipids, but also mixtures thereof, and liposomes or constituents thereof.

The dosage of the active ingredients may vary depending on the route of administration, age and weight of the patient, nature and severity of the disorder to be treated and similar factors. The treatment can be effected in single doses or as a large number of doses over a prolonged period. The daily dose is 0.5-1000 mg, preferably 50-200 mg, it being possible for the dose to be given as a single dose to be administered once or divided into 2 or more daily doses.

The above-described formulations and administration forms likewise form part of the subject matter of the present invention.

Carrier systems which can also be used are surface-active excipients such as salts of bile acids or animal or vegetable phospholipids, but also mixtures thereof, and liposomes or constituents thereof.

The inventive compounds can be administered by any conventional method including oral and parenteral methods, for example by subcutaneous or intramuscular injections. Enteral, parenteral, vaginal and oral administration likewise form part of the subject matter of the present invention.

The inventive compounds of the general formula D binds to the EP₂ receptor and have agonist or antagonistic action. It can be determined by an agonism test (see example 1.2.1 of the biological examples) or by an antagonism test (see example 1.2.2 of the biological examples) whether agonistic or antagonistic action is present.

Antagonists are understood to mean those molecules which bind to their corresponding receptors and typically compete with the naturally occurring ligand of the receptor for the binding to the receptor and which inhibit the initiation of the signal transduction pathway coupled to the receptor.

Receptor antagonists typically bind selectively to their particular receptor and not to other receptors. They normally have a higher binding affinity than the natural ligand. Even though antagonists which have a higher affinity for the receptor than the natural ligand are preferred, it is likewise possible to use antagonists with a lower affinity.

The antagonists preferably bind reversibly to their corresponding receptors.

The EP₂ receptor antagonist has a preferential affinity for the EP₂ receptor over any other EP receptor. The antagonism is measured in the presence of the natural agonist (PGE₂).

Agonists are understood to mean those molecules which bind to their corresponding receptors and typically compete with the naturally occurring ligand of the receptor for the binding to the receptor and which stimulate the initiation of the signal transduction pathway coupled to the receptor. Agonists may also promote the binding of the natural ligand.

Receptor agonists typically bind selectively to their particular receptor and not to other receptors. They normally have a higher binding affinity than the natural ligand. Even though agonists which have a higher affinity for the receptor than the natural ligand are preferred, it is likewise possible to use agonists with a lower affinity. The agonists preferably bind reversibly to their corresponding receptors.

The EP₂ receptor agonist has a preferred affinity for the EP₂ receptor over any other EP receptor.

Agonists are tested via the initiation of the signal transduction and/or physiological action mediated the corresponding receptor.

Ligands refer to the compounds or low molecular weight substances which bind to a receptor. Their binding is typically reversible. The binding of a ligand to the corresponding receptor activates or inactivates the signal transduction pathway coupled to the receptor. In this manner, the ligand imparts its intracellular action. Ligands are understood to mean agonists and antagonists of a receptor.

Substances according to examples XIII/5 or VI/4 exhibit good activity in the cellular agonism test (EC₅₀ 6×10e−6M) without any inhibition in the antagonism test (IC₅₀>2×10e−5M); see also table 1.

The present invention likewise provides for the use of the inventive substances as EP₂ receptor agonists for the treatment of disorders caused by disruptions in the signal transduction chain in which the EP₂ receptor is involved, for example endometriosis, fertility disorders, autoimmune disorders and glaucoma.

The substance according to example XIII/12 exhibits no inhibition in the cellular agonism test (EC₅₀>2×10e−5M), but good activity in the antagonism test (IC₅₀=2.4×10e−6M).

The present invention likewise provides for the use of the inventive substances as EP₂ receptor antagonists for the treatment of disorders caused by disruptions in the signal transduction chain in which the EP₂ receptor is involved, for example pain, and which are likewise suitable for fertility control.

The inventive compounds of the general formula D have profertile action. In the preovulatory antral follicle, the oocyte is surrounded by cumulus cells which form a dense ring of cells around the oocyte. After the peak of the lutenizing hormone (LH peak), a series of processes is activated and leads to a great morphological change in this ring of cumulus cells. The cumulus cells form an extracellular matrix which leads to so-called cumulus expansion (Vanderhyden et al. Dev Biol. 1990 August; 140 (2):307-317). This cumulus expansion is an important part of the ovulatory process and of the subsequent possibility of fertilization.

In cumulus expansion, prostaglandins, and here prosta-glandin E₂ whose synthesis is induced by the LH peak, are of crucial significance. Prostanoid EP₂ knockout mice (Hizaki et al., Proc Natl Acad Sci USA 1999 Aug. 31; 96(18):10501-6) exhibit markedly reduced cumulus expansion and severe subfertility, which demonstrates the significance of the prostanoid EP₂ receptor for this process.

The inventive substances have both profertile effects and inhibitory effects in cumulus expansion tests.

The present invention provides for the use of the inventive substances for the treatment of fertility disorders, such as impaired or absent ovulation, disrupted implantation and disrupted decidualization.

While the EP₂ receptor antagonist AH 6809 suppresses the expansion of the cumulus by only about 20% at a concentration of 100-200 μm, an almost 50% suppression of cumulus expansion can be achieved at the same concentration in the presence of the substance according to example VI/3. In these tests, the test substances compete with the natural EP₂ receptor agonist PGE₂.

The present invention provides for the use of the inventive substances for the inhibition of oocyte maturation for contraception.

Prostaglandins play an important role in angiogenesis (Sales, Jabbour, 2003, Reproduction 126, 559-567).

Endometriosis is a chronic disorder caused by impairments of the blood vessels. About 10% of women regularly suffer from chronic bleeding during menstruation, caused by changes in the blood vessels of the endometrium. In addition, structural differences in the blood vessels have been observed, for example incomplete formation of the smooth muscle cell layer (Abberton et al., 1999, Hum. Reprod. 14, 1072-1079). Since blood loss during menstruation is controlled partly by the constriction of the blood vessels, it is obvious that the defects in the smooth muscle structure make a substantial contribution to the bleeding.

The present invention provides for the use of the substances of the general formula D for the treatment of endometriosis.

Prostaglandins play an important role in uterus contraction; excessively strong contractions are responsible for menstrual complaints (Sales, Jabbour, 2003, Reproduction 126, 559-567).

The present invention provides for the use of the substances of the general formula D for the treatment of menstrual complaints.

EP₂ receptor agonists and antagonists also play a significant role in the regulation of the intraocular pressure. It has been shown that EP₂ receptors in particular are present in a high concentration in the vessels of the trabecular meshwork (™) of the eye. Tears leave the eye via the TM and Schlemm's canal; EP₂ receptor agonists influence the dynamics of the tear fluid by stimulating the efflux of the tear fluid and thus leads to a decrease in the intraocular pressure (W. Kamphuis et al., Current Eye Res. 2004, 29, 17-26). The present invention provides for the use of the inventive substances for the treatment of elevated intraocular pressure, as in the case of disorders including glaucoma.

Prostaglandins also play an important role in the processes which counteract osteoporosis. The present invention therefore provides for the use of the inventive substances for the treatment of osteoporosis.

The immunomodulatory action of PGE₂ has already been known for some time. For instance, it influences cytokine production in dendritic cells (DCs). IL-1β and TNF-α stimulate cytokine production (IL-12) in DCs, which results in the secretion of IL-12, and also promoted development of the type 1 T-helper cells (Th1). DCs which are stimulated by IL-1β and TNF-α in the presence of PGE₂ exhibit impaired cytokine production (IL-12) and promoted development of the type 2 T-helper cells (Th2) (Hilkens C M et al., J. Immunol. 156: 1722-1727, 1996).

Peripheral blood mononuclear cells (PBMCs) of multiple sclerosis patients require higher PGE₂ levels for the stimulation of the advantageous cytokine secretion. Dore-Duffy et al. (E. Clin. Immunol. Immunopathol. 61: 119-128, 1990) have been able to show that monocytes of MS patients reacted less sensitively to the PGE₂-mediated increases in the cAMP level (mediated by the EP₂ or EP₄ receptor). These observations led to the conclusion that MS patients require higher PGE₂ levels in order that advantageous, immunomodulatory responses can be achieved.

Ruddle et al. (J. Exp. Med. 172(4): 1193-1200, 1990) state firstly that TNF-α-producing T cells and TNF-α itself play an important role in autoimmune disorders of the central nervous system.

INF-γ promotes a deterioration in the MS (Panitch et al., J. Neuroimmunol. 46 (1-2): 155-164), so a reduction in Th-1 cytokine expression, such as that of INF-γ, should be advantageous for MS patients. The cytokine expression of Th-2 should remain unchanged thereby. PGE₂ and PGE₂ agonists ensure lowered Th-1 cytokine expression and therefore have an advantageous effect on MS patients, and are likewise suitable for the treatment of other autoimmune disorders.

The present invention provides for the use of the inventive substances for the treatment of multiple sclerosis and other autoimmune disorders.

Reinold et al. (J. Clin. Invest. 115, 673-679 (2005)) describe PGE₂ receptors of the EP₂ subtype as the key signaling elements in inflammatory hyperalgesia. Mice which no longer have this receptor (EP₂^{-/-}) experience no spinal inflammatory pain. There are indications that inflammatory, enhanced pain sensitivity can be treated by modulating EP₂ receptors in a controlled manner.

The present invention provides for the use of the inventive substances for the treatment of inflammatory hyperalgesia.

Where the preparation of the starting compounds is not described, these can be prepared in a known manner or analogously to known compounds or processes described here. It is likewise possible to perform all reactions described here in parallel reactions or by means of combinatorial techniques.

The isomer mixtures can be separated into the enantiomers or E/Z isomers by customary methods, for example crystallization, chromatography or salt formation.

The salts are prepared in a customary manner by admixing a solution of the compound of the formula D with the equivalent amount or an excess of a base or acid, which may be in solution, and removing the precipitate or working up the solution in a customary manner.

The invention thus also relates to medicaments based on compounds of the general formula D and the customary excipients or carriers.

The present invention also further relates to processes for preparing the compounds of the general formula D, and in each case to processes for preparing the individual intermediates A to C: 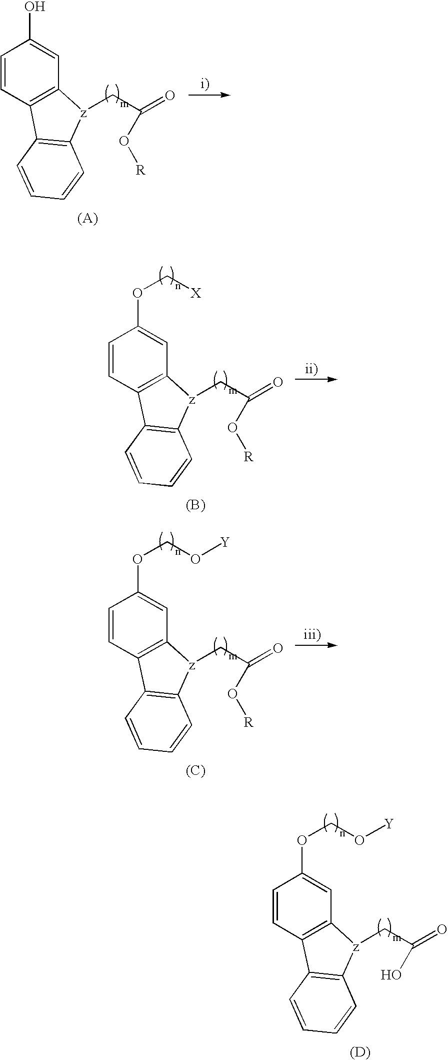
in which the X, Y, Z, R, m and n radicals are each as defined in the general formula D. Compounds A-C find use as intermediates in the preparation process to give the compounds of the general formula D:
i) 1, n-dibromoalkane/potassium carbonate
ii) compound of the general formula E/potassium carbonate
iii) 1M sodium hydroxide/ethanol

The inventive compounds of the general formula D can be prepared as described in the examples. Analogous procedure using homologous reagents to the reagents described in the examples allows the further compounds of the general formula D to be obtained. Substituents according to the general formula D can, though, instead of the described alkylation of an aromatic alcohol with an alkyl bromide, also equally be introduced by means of Mitsunobu reaction between aromatic alcohol and the appropriate aliphatic alcohol.

Substituents according to the general formula D can also be introduced at the stage of the general formula A. This may be viable and more effective especially in the case of the selective synthesis of a desired end compound.

When Z=CH, the inventive compounds may be present in the form of α,β-stereoisomers. In the case of the preparation of the compounds by the processes described, the compounds are obtained as mixtures of the corresponding α,β-isomers. The mixtures can be separated, for example, by chromatographic processes.

Starting material used for such syntheses is 2-hydroxy-9-fluorenone in the case of Z=CH, and 2-hydroxycarbazole when Z=N. 2-Hydroxyfluorenone can be converted to a compound of the general formula A in a reaction with triethyl phosphonoacetate, and 2-hydroxycarbazole in a reaction sequence composed of O-benzylation, N-alkylation and debenzylation.

The carboxylic esters initially obtained in this way, and also carboxylic acids, can be converted further to a variety of functional groups by methods known to those skilled in the art.

Frequently used abbreviations:
THF tetrahydrofuran
KOtBu potassium tert-butoxide
EA ethyl acetate
Cx cyclohexane
DMF N,N-dimethylformamide
MTBE tert-butyl methyl ether
Equiv. equivalents
HOBT 1-hydroxybenzotriazole
EDC N-(3-dimethylaminopropyl)-N′-ethylcarbodiimide
DMAP 4-dimethylaminopyridine

The products are characterized by means of GC-MS (column: HP5MS (30 m×0.25 mm×0.25 μm); T (injector): 310° C., T (transfer line): 310° C., temperature program: 60° C./10 min, then 50° C./min to 310° C., then 310° C./45 min; flow rate: 0.7 ml/min; 12 psi/220° C., split: 0.1 min: 24 ml, constant flow), or LC-MS (column: HP Hypersil ODS (5 μm, 4.6×250 mm); eluent 1: 90:10 acetonitrile/2 mM aqueous ammonium acetate solution, eluent 2: 80:20 acetonitrile/2 mM aqueous ammonium acetate solution, eluent 3: 80:20 acetonitrile/water+0.5% formic acid, eluent 4: 60:40 acetonitrile/water+0.5% formic acid, eluent 5: 70:30 acetonitrile/water+0.5% formic acid, eluent 6: 85:15 acetonitrile/2 mM aqueous ammonium acetate solution, eluent 7: 70:30 acetonitrile/2 mM aqueous ammonium acetate solution, eluent 8: 50:50 acetonitrile/2 mM aqueous ammonium acetate solution; flow rate: 1.0 ml/min; APCI positive).

### Synthesis Schemes

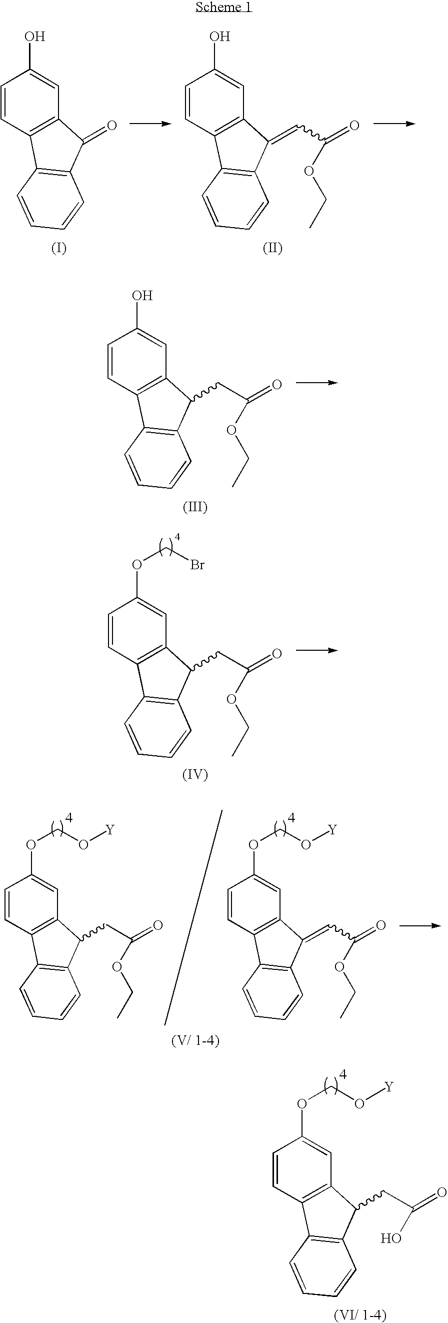 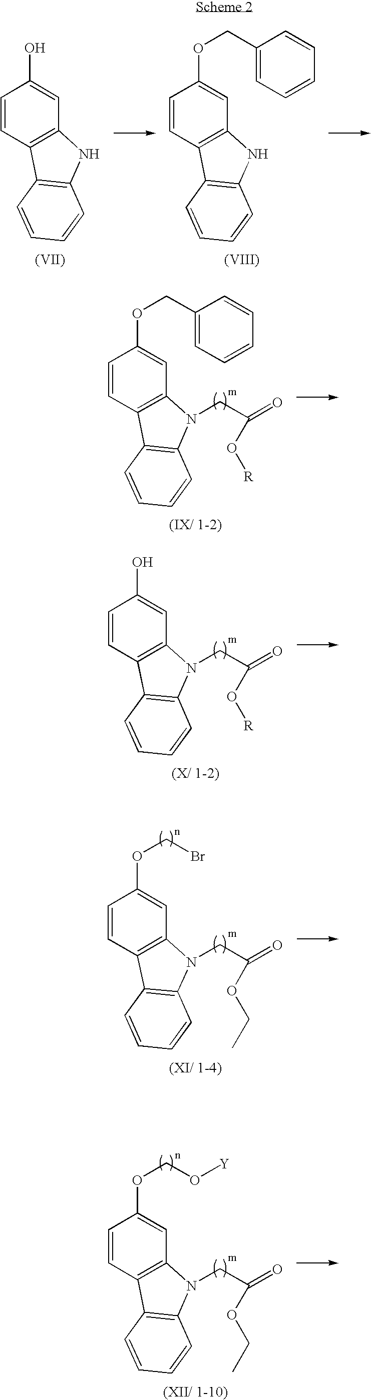 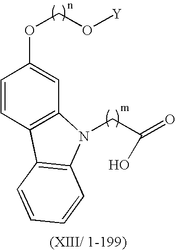 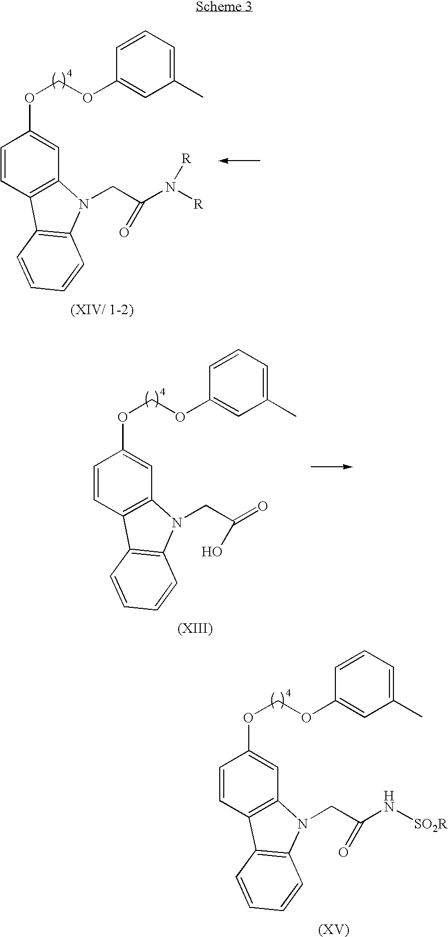 General Working Method 1: Introduction of the Bromo-Alkyl Side Chains

The appropriate 1,n-dibromoalkane (5 equiv.) and potash (10 equiv.) are added at room temperature to a solution of the appropriate phenol in DMF (3 ml/mmol). Subsequently, the mixture is stirred at room temperature until conversion is complete. Water is added, and the mixture is extracted repeatedly with MTBE, washed with water and sat. sodium chloride solution, dried over sodium sulfate and concentrated. The residues are purified by column chromatography (Cx/EA) and give rise to the corresponding alkyl bromides.

General Working Method 2: Aryl Ether Synthesis

The appropriate phenol (2.2 equiv.) and potash (8 equiv.) are added at room temperature to a solution of the appropriate alkyl bromide in DMF (5 ml/mmol). Subsequently, the mixture is stirred at room temperature until conversion is complete. Water is added, and the mixture is extracted repeatedly with EA, washed with water and sat. sodium chloride solution, dried over sodium sulfate and concentrated. The residues are purified by column chromatography (Cx/EA) and give rise to the corresponding aryl ethers.

General Working Method 3: Ester Hydrolysis

1M sodium hydroxide solution (15 ml/mmol) is added at room temperature to a solution of the appropriate ester in ethanol (15 ml/mmol). Subsequently, the mixture is stirred at room temperature until conversion is complete. Water is added, and the mixture is neutralized with 1M hydrochloric acid, extracted repeatedly with EA, washed with sat. sodium chloride solution, dried over sodium sulfate and concentrated. Further purification of the carboxylic acids thus obtained is not required.

In the case of the fluorenyl-/fluorenylideneacetic acids, the residue is dissolved in a sufficient amount of THF, admixed with palladium (10% on activated carbon, 0.5 mg/mg), and stirred under a hydrogen atmosphere until conversion is complete. Subsequently, the mixture is filtered through Celite and concentrated. The residues are purified by column chromatography (Cx/EA) and give rise to the corresponding carboxylic acids.

General Working Method 4: N-Alkylation

An appropriate amount of carbazole VIII is initially charged in DMF (6 ml/mmol) and cooled to 0° C. Subsequently, sodium hydride (55%, 3 equiv.) is added in portions and, 5 minutes later, the appropriate ethyl ω-halocarboxylate (3 equiv.). The mixture is stirred at 0° C. until conversion is complete. For workup, sat. ammonium chloride solution is added dropwise and the mixture is stirred for 15 minutes. Subsequently, water is added, and the mixture is extracted repeatedly with EA, washed with water and sat. sodium chloride solution, dried over sodium sulfate and concentrated. The residues are purified by column chromatography (Cx/EA) and give rise to the corresponding N-alkylated carbazoles.

General Working Method 5: Benzyl Ether Hydrogenolysis

A solution of the appropriate benzyl ether in THF (5 ml/mmol) is admixed with palladium (10% on activated carbon, 0.2 g/g) and stirred under a hydrogen atmosphere until conversion is complete. Subsequently, the mixture is filtered through Celite and concentrated. The phenols thus obtained are used in the next stage without further purification.

General Working Method 6: Synthesis of the Carboxylic Acids XIII/11-199 Starting From the Intermediates X/1-4

Potassium carbonate (10 equiv.) is initially charged, a solution of the appropriate phenol (0.4M in DMF; 2 equiv.) is added, the appropriate bromide solution X/1-4 (0.2M in DMF, 0.1 mmol) is added, the mixture is stirred at 80° C. for 10 h and diluted with MTBE (3.0 ml), water is added (1.5 ml), the mixture is extracted, the aqueous phase is discarded and the organic phase is concentrated. A potassium hydroxide solution (0.5M in 3/1/1 ethanol/THF/water; 10 equiv.) is added to the residue, the mixture is stirred at 60° C. for 10 h, the reaction mixture is concentrated and purified by means of preparative HPLC-MS, and the products are characterized by means of analytical HPLC-MS MS (column: LiChroCart Purospher (125×4.5 mm, RP18e, 5 μm); gradient 5-95% acetonitrile (0.1% trifluoroacetic acid) in water (0.1% trifluoroacetic acid) (15 min); flow rate 1.0 ml/min; MS ES+).

General Working Method 7: Synthesis of the Amides XIV from the Carboxylic Acids XIII

An appropriate amount of carboxylic acid XIII is initially charged in DMF (8 ml/mmol), admixed with HOBt (2 equiv.) and EDC (2 equiv.) and stirred at room temperature for 30 minutes. Subsequently, the appropriate amine (2 equiv.) is added and the mixture is stirred at room temperature until conversion is complete. For workup, the reaction solution is stirred into water, extracted repeatedly with EA, neutralized, washed with sat. sodium chloride solution and concentrated. The residues are recrystallized from dichloromethane/methanol and give rise to the corresponding amides XIV.

General Working Method 8: Synthesis of the Sulfonamides XV from the Carboxylic Acids XIII

An appropriate amount of carboxylic acid XIII is initially charged in dichloromethane (80 ml/mmol), admixed with EDC (2 equiv.), the corresponding sulfon-amide (2 equiv.) and DMAP (2 equiv.), and stirred at room temperature until conversion is complete. For workup, the mixture is diluted with water, extracted repeatedly with dichloromethane, neutralized, washed with sat. sodium chloride solution and concentrated. The residues are purified by column chromatography (Cx/EA) and give rise to the corresponding sulfonamides XV. The sulfonamides are characterized by means of LC-MS (column: Zorbax Extend C-18, 3.0 mm ID×50 mm, 3.5 micron).

The examples which follow serve to further illustrate the invention.

### (E/Z) Ethyl (2-hydroxyfluoren-9-ylidene)acetate (II)

20 ml of triethyl phosphonoacetate and then, in portions, 11.3 g of KOtBu are added at 15° C. to a solution of 4.8 g of the ketone I in 150 ml of THF. The mixture is stirred under reflux until conversion is complete. After cooling to room temperature, the mixture is admixed with 10% ammonium chloride solution and stirred for a further 30 minutes. It is extracted repeatedly with EA, washed with water and sat. sodium chloride solution, dried over sodium sulfate and concentrated. The residue is purified by column chromatography (Cx/EA) and gives rise to 1.6 g of olefin II (GC-MS: m/z theor.: 266, actual M⁺: 266, TR: 16.27 and 16.43 min).

### (R/S) Ethyl (2-hydroxy-9H-fluoren-9-yl)acetate (III)

300 mg of palladium (10% on activated carbon) are added to a solution of 1.6 g of olefin II in 25 ml of THF/ethanol (4:1). The mixture is stirred under a hydrogen atmosphere at room temperature until conversion is complete. Subsequently, the mixture is filtered through Celite and concentrated. The resulting ester III (GC-MS: m/z theor.: 268, actual M⁺: 268, TR: 15.77 min) is used in the next reaction without further purification.

### (R/S) Ethyl [2-(4-bromobutoxy)-9H-fluoren-9-yl]acetate (IV)

800 mg of phenol III, in the reaction with 1,4-dibromobutane analogous to general working method 1, give rise to 1940 mg of alkyl bromide IV (GC-MS: m/z theor. M⁺: 402, actual M⁺: 402, TR: 17.62 min).

### (E/Z) Ethyl [2-(4-m-tolyloxybutoxy)fluorenylidene]-acetate (V/1)

200 mg of alkyl bromide IV, in the reaction with m-cresol analogous to general working method 2, give rise to 103 mg of aryl ether V/1 as the α,β-unsaturated ester (LC-MS: eluent 1, m/z theor. (M+H)⁺: 429, actual (M+H)⁺: 429, TR: 8.65 and 9.44 min.

### (R/S) Ethyl {2-[4-(3-hydroxy-5-methoxyphenoxy)butoxy]-9H-fluoren-9-yl}acetate (V/2)

200 mg of alkyl bromide IV, in the reaction with 5-methoxyresorcinol analogous to general working method 2, give rise to 84 mg of aryl ether V/2 as a mixture of saturated and unsaturated compound (LC-MS: eluent 2, m/z theor. (M+H)⁺: 461/463, actual (M+H)⁺: 461/463, TR: 7.17 and 7.98/5.63 min).

### (R/S) Ethyl {2-[4-(3-hydroxyphenoxy)butoxy]-9H-fluoren-9-yl}acetate (V/3)

200 mg of alkyl bromide IV, in the reaction with resorcinol analogous to general working method 2, give rise to 132 mg of aryl ether V/3 as a mixture of saturated and unsaturated compound (LC-MS: eluent 2, m/z theor. (M+H)⁺: 431/433, actual (M+H)⁺: 431/433, 7.48 and 8.36/5.83 min).

### (R/S) Ethyl {2-[4-(3-acetylaminophenoxy)butoxy]-9H-fluoren-9-yl}acetate (V/4)

206 mg of alkyl bromide IV, in the reaction with 3-acetamidophenol analogous to general working method 2, give rise to 205 mg of aryl ether V/v as a mixture of saturated and unsaturated compound (LC-MS: eluent 2, m/z theor. (M+H)⁺: 472/474, actual: 472/474, TR: 7.09 and 7.84/5.56 min).

### (R/S) [2-(4-m-Tolyloxybutoxy)-9H-fluoren-9-yl]acetic acid (VI/1)

100 mg of ester V/1, in the reaction analogous to general working method 3, give rise to 34 mg of acid VI/1 (LC-MS: eluent 3, m/z theor. (M+H)⁺: 403, actual (M+H)⁺: 403, TR: 10.46 min)

### (R/S) {2-[4-(3-Hydroxy-5-methoxyphenoxy)butoxy]-9H-fluoren-9-yl}acetic acid (VI/2)

80 mg of ester V/2, in the reaction analogous to general working method 3, give rise to 57 mg of acid VI/2 (LC-MS: eluent 4, m/z theor. (M+H)⁺: 435, actual (M+H)⁺: 435, TR: 12.98 min)

### (R/S) {2-[4-(3-Hydroxyphenoxy)butoxy]-9H-fluoren-9-yl}acetic acid (VI/3)

130 mg of ester V/3, in the reaction analogous to general working method 3, give rise to 71 mg of acid VI/3 (LC-MS: eluent 4, m/z theor. (M+H)⁺: 405, actual (M+H)⁺: 405, TR: 13.28 min).

### (R/S) {2-[4-(3-Acetylaminophenoxy)butoxy]-9H-fluoren-9-yl}acetic acid (VI/4)

136 mg of ester V/4, in the reaction analogous to general working method 3, give rise to 76 mg of acid VI/4 (LC-MS: eluent 4, m/z theor. (M+H)⁺: 446, actual (M+H)⁺: 446, TR: 12.09 min).

### 2-Benzyloxy-9H-carbazole (VIII)

18.9 g of potash and 8.1 ml of benzyl bromide are added at room temperature to a solution of 5 g of 9H-carbazol-2-ol (VII) in 60 ml of DMF. The mixture is stirred at room temperature until conversion is complete. Water is added, and the mixture is extracted repeatedly with dichloromethane, washed with sat. ammonium chloride and sat. sodium chloride solution, dried over sodium sulfate and concentrated. The residue is purified by column chromatography (Cx/EA) and gives rise to 4 g of benzyl ether VIII (GC-MS: m/z theor. M⁺: 273, actual M⁺: 273, TR: 18.44 min)

### Ethyl (2-benzyloxycarbazol-9-yl)acetate (IX/1)

2.2 g of carbazole VIII, in the reaction with ethyl chloroacetate analogous to general working method 4, give rise to 2.9 g of acid IX/1 (GC-MS: m/z theor. M⁺: 359, actual M⁺: 359, TR: 19.65 min).

### Ethyl 3-(2-benzyloxycarbazol-9-yl)propionate (IX/2)

2.2 g of carbazole VIII, in the reaction with ethyl bromopropionate analogous to general working method 4, give rise to 2.8 g of acid IX/2 (GC-MS: m/z theor. M⁺: 373, actual M⁺: 373, TR: 20.78 min)

### Ethyl (2-hydroxycarbazol-9-yl)acetate (X/1)

2.9 g of benzyl ether IX/1, in the reaction analogous to general working method 5, give rise to 2.2 g of phenol X/1 (GC-MS: m/z theor. M⁺: 269, actual M⁺: 269, TR: 16.65 min).

### Ethyl 3-(2-hydroxycarbazol-9-yl)propionate (X/2)

2.8 g of benzyl ether IX/2, in the reaction analogous to general working method 5, give rise to 2.1 g of phenol X/2 (GC-MS: m/z theor. M⁺: 283, actual M⁺: 283, TR: 17.10 min).

### Ethyl [2-(3-bromopropoxy)carbazol-9-yl]acetate (XI/1)

2.8 g of phenol X/1, in the reaction with 1,3-dibromopropane analogous to general working method 1, give rise to 2.5 g of alkyl bromide XI/1 (LC-MS: eluent 2, m/z theor. (M+H)⁺: 390, actual (M+H)⁺: 390, TR: 5.33 min).

### Ethyl [2-(4-bromobutoxy)carbazol-9-yl]acetate (XI/2)

2.3 g of phenol X/1, in the reaction with 1,4-dibromobutane analogous to general working method 1, give rise to 2.9 g of alkyl bromide XI/2 (GC-MS: m/z theor. M⁺: 403, actual M⁺: 403, TR: 19.41 min).

### Ethyl [2-(5-bromopentyloxy)carbazol-9-yl]acetate (XI/3)

2.8 g of phenol X/1, in the reaction with 1,5-dibromopentane analogous to general working method 1, give rise to 3.8 g of alkyl bromide XI/3 (LC-MS: eluent 2, m/z theor. (M+H)⁺: 418, actual (M+H)⁺: 418, TR: 6.81 min).

### Ethyl 3-[2-(4-bromobutoxy)carbazol-9-yl]propionate (XI/4)

2.1 g of phenol X/2, in the reaction with 1,4-dibromobutane analogous to general working method 1, give rise to 2.5 g of alkyl bromide XI/4 (GC-MS: m/z theor. M⁺: 417, actual M⁺: 417, TR: 20.47 min).

### Ethyl {2-[4-(3-hydroxyphenoxy)butoxy]carbazol-9-yl}acetate (XII/1)

200 mg of alkyl bromide XI/2, in the reaction with resorcinol analogous to general working method 2, give rise to 100 mg of aryl ether XII/1 (LC-MS: eluent 2, m/z theor. (M+H)⁺: 434, actual (M+H)⁺: 434, TR: 4.62 min).

### Ethyl [2-(4-m-tolyloxybutoxy)carbazol-9-yl]acetate (XII/2)

227 mg of alkyl bromide XI/2, in the reaction with m-cresol analogous to general working method 2, give rise to 141 mg of aryl ether XII/2 (GC-MS: m/z theor. M⁺: 431, actual M⁺: 431, TR: 26.19 min).

### Ethyl {2-[4-(3-acetylaminophenoxy)butoxy]carbazol-9-yl}acetate (XII/3)

204 mg of alkyl bromide XI/2, in the reaction with 3-acetamidophenol analogous to general working method 2, give rise to 104 mg of aryl ether XII/3 (LC-MS: eluent 5, m/z theor. (M+H)⁺: 475, actual (M+H)⁺: 475, TR: 6.92 min).

### Ethyl {2-[4-(3-hydroxy-5-methoxyphenoxy)butoxy]carbazol-9-yl}acetate (XII/4)

218 mg of alkyl bromide XI/2, in the reaction with 5-methoxyresorcinol analogous to general working method 2, give rise to 96 mg of aryl ether XII/4 (LC-MS: eluent 2, m/z theor. (M+H)⁺: 464, actual (M+H)⁺: 464, TR: 4.51 min).

### Methyl 3-[4-(9-ethoxycarbonylmethyl-9H-carbazol-2-yloxy)butoxy]benzoate (XII/5)

216 mg of alkyl bromide XI/2, in the reaction with methyl 3-hydroxybenzoate analogous to general working method 2, give rise to 195 mg of aryl ether XII/5 (GC-MS: m/z theor. M⁺: 475, actual M⁺: 475, TR: 34.72 min).

### Ethyl 3-{2-[4-(3-hydroxyphenoxy)butoxy]carbazol-9-yl}-propionate (XII/6)

206 mg of alkyl bromide XI/4, in the reaction with resorcinol analogous to general working method 2, give rise to 101 mg of aryl ether XII/6 (LC-MS: eluent 6, m/z theor. (M+H)⁺: 448, actual (M+H)⁺: 448, TR: 4.32 min).

### Ethyl 3-[2-(4-m-tolyloxybutoxy)carbazol-9-yl]propionate (XII/7)

211 mg of alkyl bromide XI/4, in the reaction with m-cresol analogous to general working method 2, give rise to 177 mg of aryl ether XII/7 (GC-MS: m/z theor. M⁺: 445, actual M⁺: 445, TR: 28.87 min).

### Ethyl 3-{2-[4-(3-acetylaminophenoxy)butoxy]carbazol-9-yl}propionate (XII/8)

214 mg of alkyl bromide XI/4, in the reaction with 3-acetamidophenol analogous to general working method 2, give rise to 162 mg of aryl ether XII/8 (LC-MS: eluent 7, m/z theor. (M+H)⁺: 489, actual (M+H)⁺: 489, TR: 8.47 min).

### Ethyl 3-{2-[4-(3-hydroxy-5-methoxyphenoxy)butoxy]-carbazol-9-yl}propionate (XII/9)

206 mg of alkyl bromide XI/4, in the reaction with 5-methoxyresorcinol analogous to general working method 2, give rise to 109 mg of aryl ether XII/9 (LC-MS: eluent 6, m/z theor. (M+H)⁺: 478, actual (M+H)⁺: 478, TR: 4.26 min).

### Methyl 3-{4-[9-(2-ethoxycarbonylethyl)-9H-carbazol-2-yloxy]butoxy}benzoate (XII/10)

218 mg of alkyl bromide XI/4, in the reaction with methyl 3-hydroxybenzoate analogous to general working method 2, give rise to 223 mg of aryl ether XII/10 (GC-MS: m/z theor. M⁺: 489, actual M⁺: 489, TR: 40.05 min)

### {2-[4-(3-Hydroxyphenoxy)butoxy]carbazol-9-yl}acetic acid (XIII/1)

100 mg of ester XII/1, in the reaction analogous to general working method 3, give rise to 84 mg of acid XIII/1 (LC-MS: eluent 5, m/z theor. (M+H)⁺: 406, actual (M+H)⁺: 406, TR: 4.39).

### [2-(4-m-tolyloxybutoxy)carbazol-9-yl]acetic acid (XIII/2)

141 mg of ester XII/2, in the reaction analogous to general working method 3, give rise to 131 mg of acid XIII/2 (LC-MS: eluent 4, m/z theor. (M+H)⁺: 404, actual (M+H)⁺: 404, TR: 21.07 min).

### {2-[4-(3-Acetylaminophenoxy)butoxy]carbazol-9-yl}acetic acid (XIII/3)

104 mg of ester XII/3, in the reaction analogous to general working method 3, give rise to 91 mg of acid XIII/3 (LC-MS: eluent 4, m/z theor. (M+H)⁺: 447, actual (M+H)⁺: 447, TR: 6.33 min).

### {2-[4-(3-Hydroxy-5-methoxyphenoxy)butoxy]carbazol-9-yl}acetic acid (XIII/4)

96 mg of ester XII/4, in the reaction analogous to general working method 3, give rise to 87 mg of acid XIII/4 (LC-MS: eluent 4, m/z theor. (M+H)⁺: 436, actual (M+H)⁺: 436, TR: 6.83 min)

### 3-[4-(9-Carboxymethyl-9H-carbazol-2-yloxy)butoxy]-benzoic acid (XIII/5)

195 mg of ester XII/5, in the reaction analogous to general working method 3, give rise to 167 mg of acid XIII/5 (LC-MS: eluent 4, m/z theor. (M+H)⁺: 434, actual (M+H)⁺: 434, TR: 6.80 min).

### 3-{2-[4-(3-Hydroxyphenoxy)butoxy]carbazol-9-yl}-propionic acid (XIII/6)

82 mg of ester XII/6, in the reaction analogous to general working method 3, give rise to 61 mg of acid XIII/6 (LC-MS: eluent 5, m/z theor. (M+H)⁺: 420, actual (M+H)⁺: 420, TR: 4.81 min)

### 3-[2-(4-m-Tolyloxybutoxy)carbazol-9-yl]propionic acid (XIII/7)

177 mg of ester XII/7, in the reaction analogous to general working method 3, give rise to 166 mg of acid XIII/7 (LC-MS: eluent 5, m/z theor. (M+H)⁺: 418, actual (M+H)⁺: 418, TR: 11.52 min).

### 3-{2-[4-(3-Acetylaminophenoxy)butoxy]carbazol-9-yl}propionic acid (XIII/8)

126 mg of ester XII/8, in the reaction analogous to general working method 3, give rise to 116 mg of acid XIII/8 (LC-MS: eluent 5, m/z theor. (M+H)⁺: 461, actual (M+H)⁺: 461, TR: 4.66 min).

### 3-{2-[4-(3-Hydroxy-5-methoxyphenoxy)butoxy]carbazol-9-yl}propionic acid (XIII/9)

109 mg of ester XII/9, in the reaction analogous to general working method 3, give rise to 82 mg of acid XIII/9 (LC-MS: eluent 5, m/z theor. (M+H)⁺: 450, actual (M+H)⁺: 450, TR: 4.83 min).

### 3-{4-[9-(2-Carboxyethyl)-9H-carbazol-2-yloxy]butoxy}-benzoic acid (XIII/10)

253 mg of ester XII/10, in the reaction analogous to general working method 3, give rise to 162 mg of acid XIII/10 (LC-MS: eluent 5, m/z theor. (M+H)⁺: 448, actual (M+H)⁺: 448, TR: 4.88 min)

### N-Ethyl-2-[2-(4-m-tolyloxybutoxy)carbazol-9-yl]acetamide (XIV/1)

100 mg of carboxylic acid XIII/2, in the reaction with ethylamine analogous to general working method 7, give rise to 104 mg of amide XIV/1 (GC-MS: m/z theor. M⁺: 430, actual M⁺: 430, TR: 27.75 min)

### N,N-Diethyl-2-[2-(4-m-tolyloxybutoxy)carbazol-9-yl]acetamide (XIV/2)

100 mg of carboxylic acid XIII/2, in the reaction with diethylamine analogous to general working method 7, give rise to 109 mg of amide XIV/2 (GC-MS: m/z theor. M⁺: 458, actual M⁺: 458, TR: 32.78 min)

### N-{2-[2-(4-m-Tolyloxybutoxy)carbazol-9-yl]acetyl}methanesulfonamide (XV/1)

51 mg of carboxylic acid XIII/2, in the reaction with methanesulfonamide analogous to general working method 8, give rise to 49 mg of sulfonamide XV/1 (LC-MS: eluent 8, m/z theor. (M+H)⁺: 481, actual (M+H⁺) 481, TR: 0.72 min).

| Example | Structure XIII/1-10 |
|---|---|
| 1 | 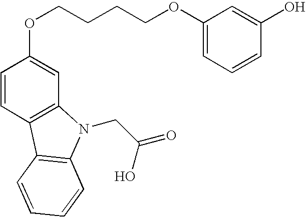 |
| 2 | 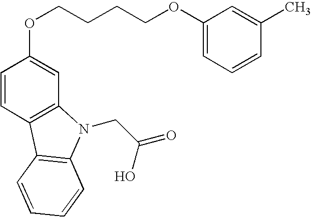 |
| 3 | 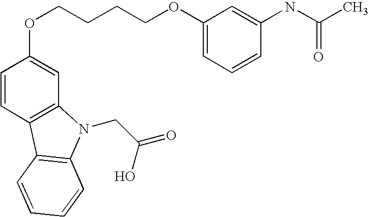 |
| 4 | 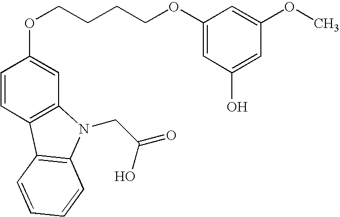 |
| 5 | 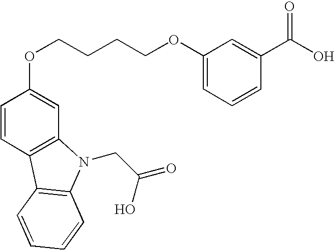 |
| 6 | 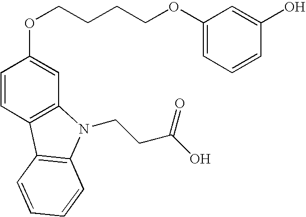 |
| 7 | 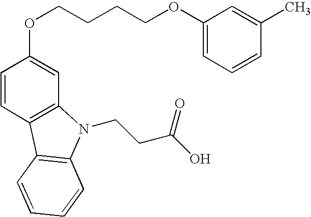 |
| 8 | 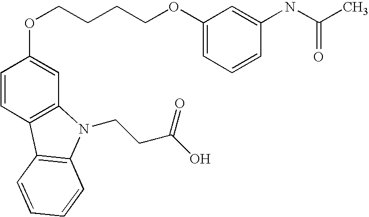 |
| 9 | 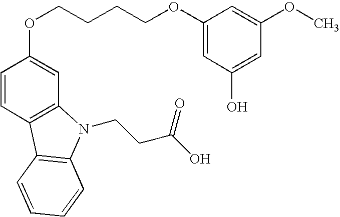 |
| 10 | 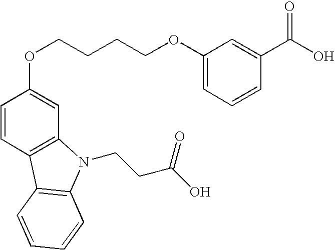 |

| Substance | | | | |
|---|---|---|---|---|
| according | | | | |
| to example | | MW | MW | RT |
| XIII/11-199 | Structure | calc. | ES+ | (min.) |
| 11 | 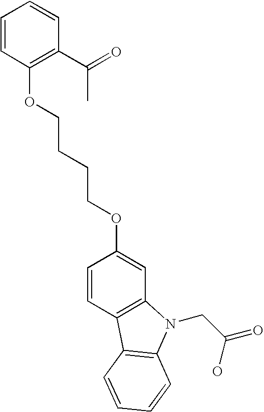 | 431 | 431 | 10.28 |
| 12 | 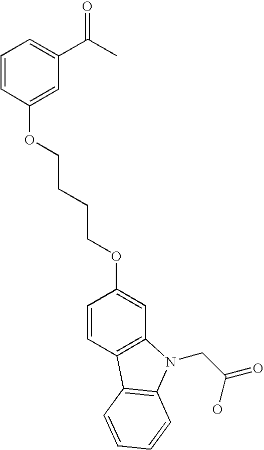 | 431 | 431 | 10.25 |
| 13 | 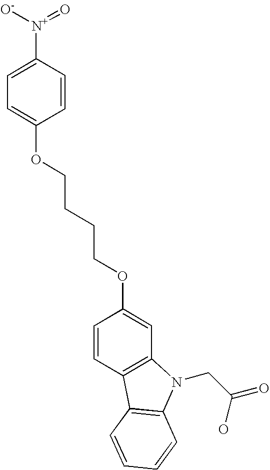 | 434 | 434 | 10.61 |
| 14 | 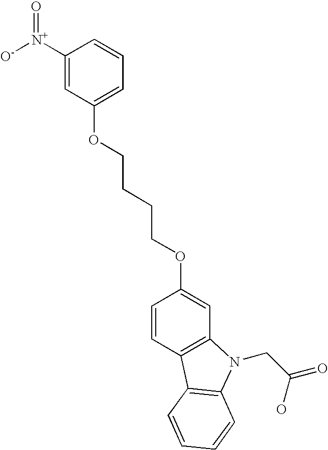 | 434 | 434 | 10.73 |
| 15 | 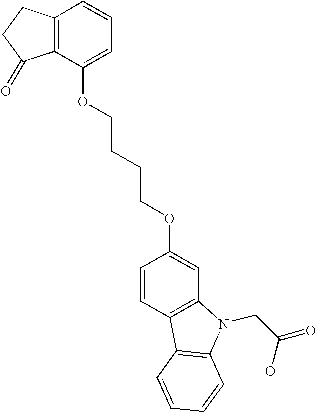 | 444 | 443 | 9.85 |
| 16 | 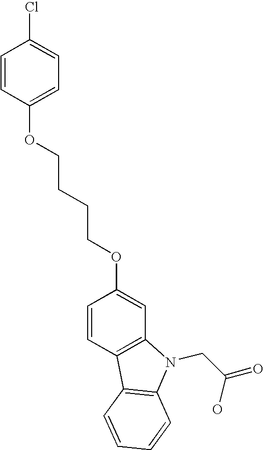 | 424 | 423 | 11.19 |
| 17 | 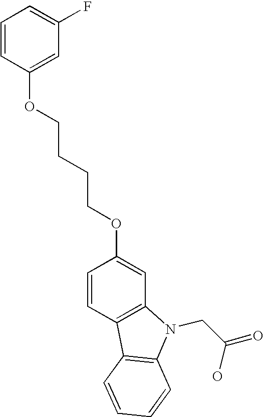 | 407 | 407 | 10.90 |
| 18 | 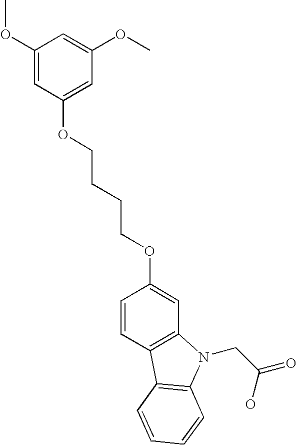 | 450 | 449 | 10.54 |
| 19 | 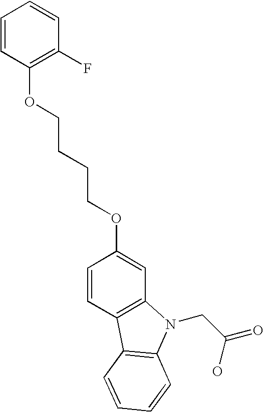 | 407 | 407 | 10.67 |
| 20 | 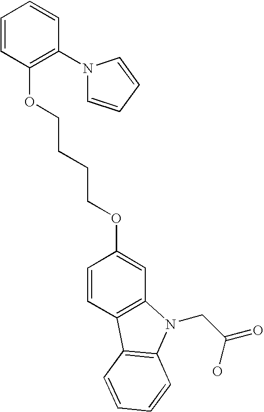 | 455 | 454 | 11.16 |
| 21 | 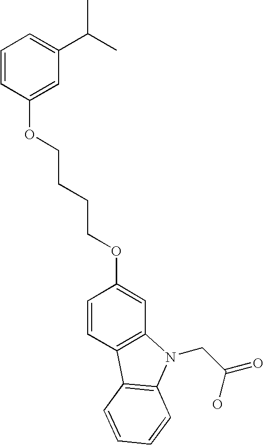 | 432 | 431 | 11.77 |
| 22 | 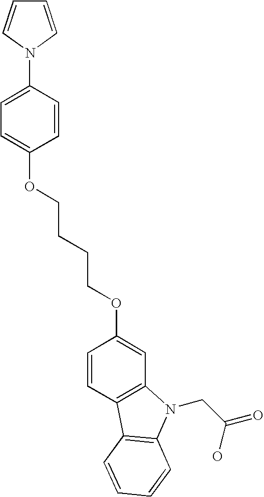 | 455 | 454 | 11.25 |
| 23 | 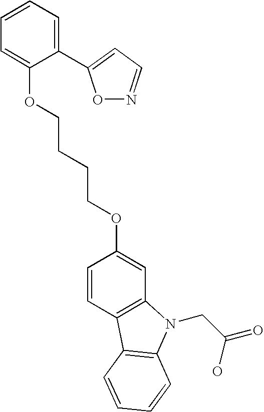 | 457 | 456 | 9.96 |
| 24 | 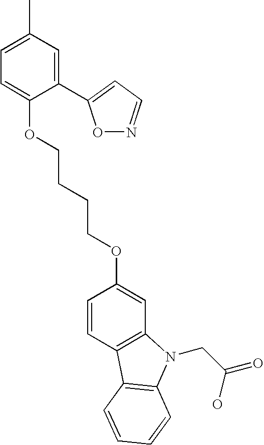 | 471 | 470 | 10.32 |
| 25 | 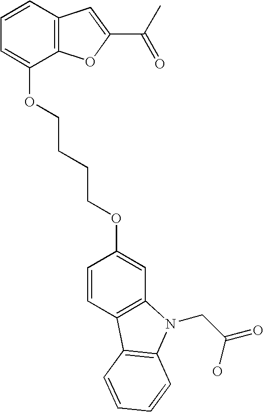 | 472 | 471 | 10.41 |
| 26 | 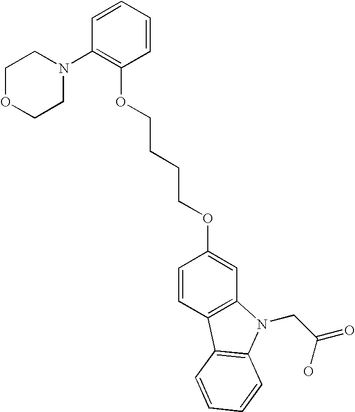 | 475 | 474 | 8.02 |
| 27 | 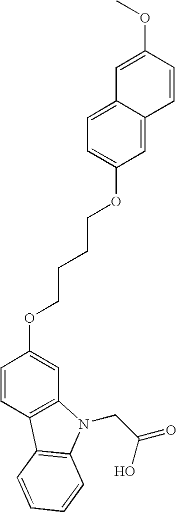 | 470 | 469 | 11.29 |
| 28 | 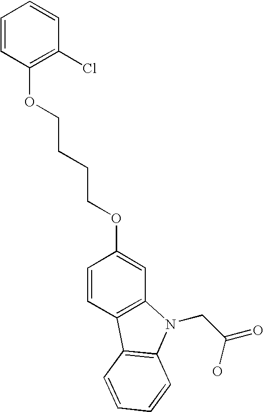 | 424 | 423 | 11.09 |
| 29 | 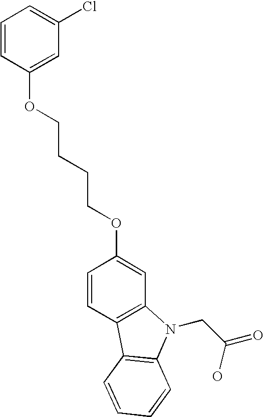 | 424 | 423 | 11.37 |
| 30 | 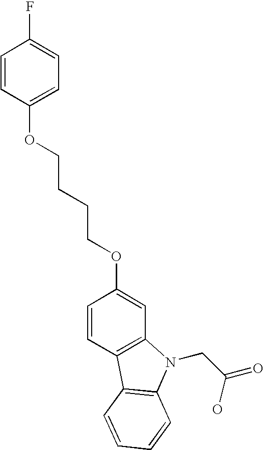 | 407 | 407 | 10.69 |
| 31 | 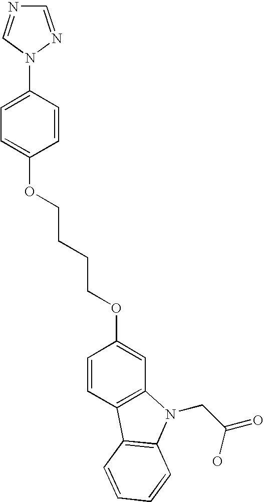 | 457 | 456 | 9.49 |
| 32 | 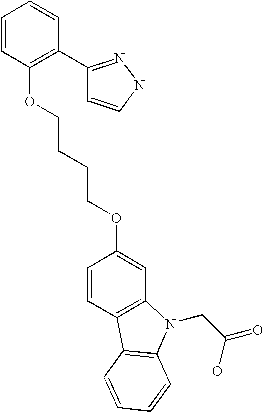 | 456 | 455 | 9.19 |
| 33 | 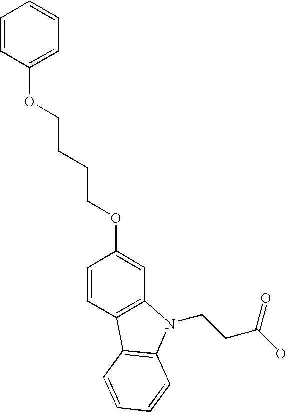 | 403 | 403 | 11.12 |
| 34 | 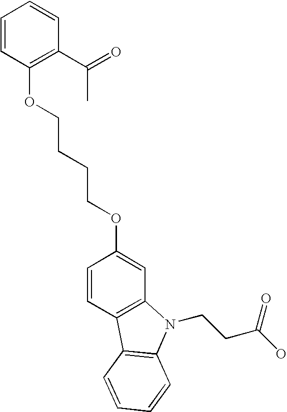 | 446 | 445 | 10.62 |
| 35 | 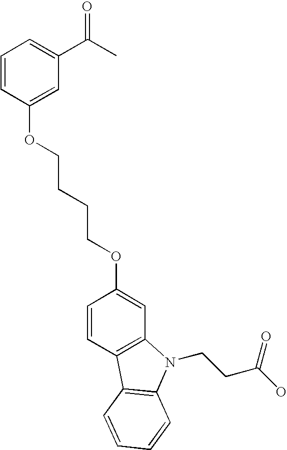 | 446 | 445 | 10.68 |
| 36 | 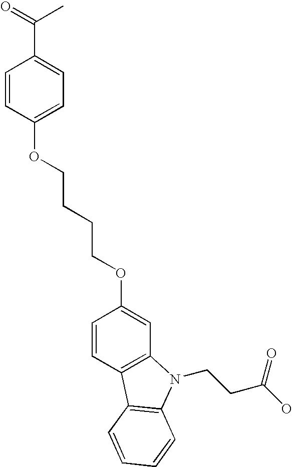 | 446 | 445 | 10.52 |
| 37 | 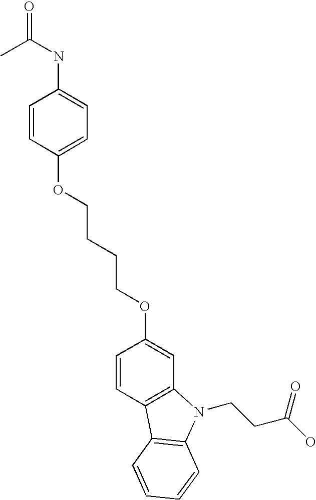 | 461 | 460 | 9.37 |
| 38 | 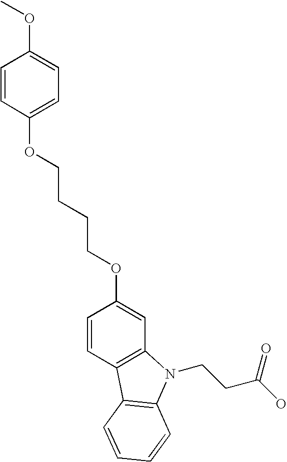 | 434 | 433 | 10.84 |
| 39 | 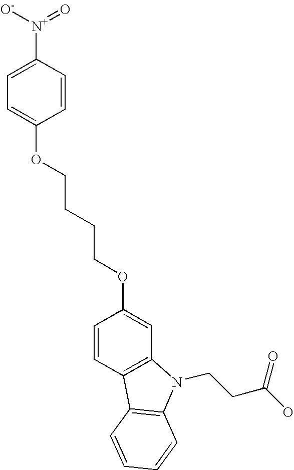 | 448 | 448 | 10.96 |
| 40 | 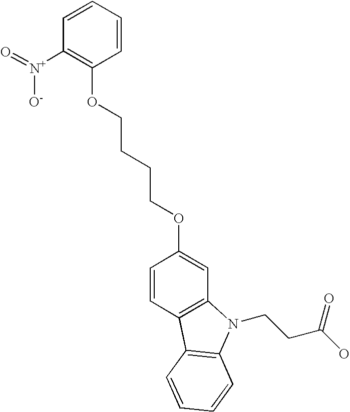 | 448 | 448 | 10.52 |
| 41 | 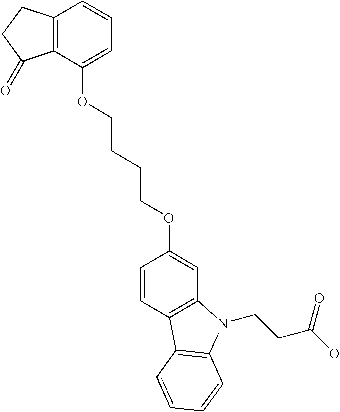 | 458 | 457 | 10.18 |
| 42 | 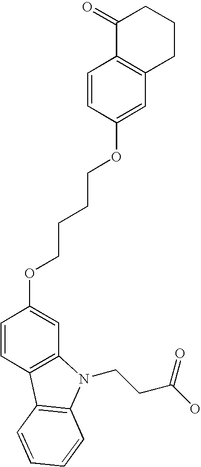 | 472 | 471 | 10.84 |
| 43 | 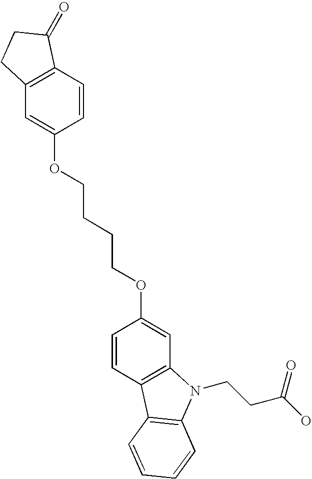 | 458 | 457 | 10.32 |
| 44 | 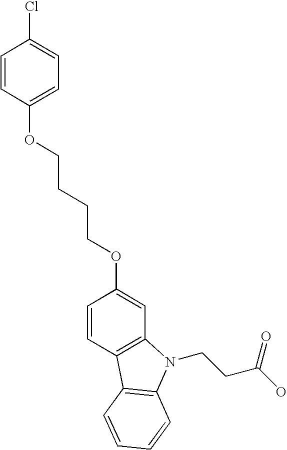 | 438 | 437 | 11.54 |
| 45 | 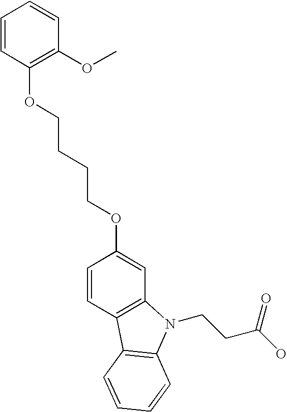 | 434 | 433 | 10.68 |
| 46 | 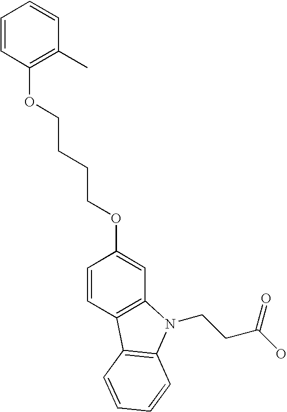 | 418 | 417 | 11.69 |
| 47 | 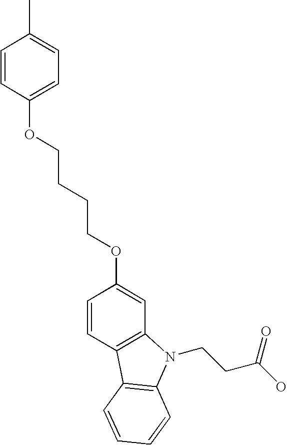 | 418 | 417 | 11.42 |
| 48 | 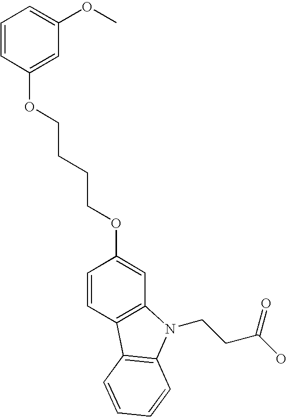 | 434 | 433 | 11.07 |
| 49 | 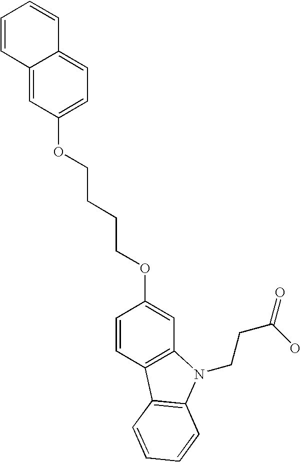 | 454 | 453 | 11.91 |
| 50 | 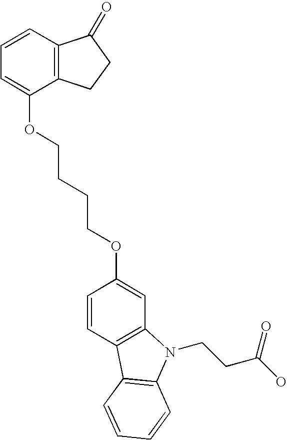 | 458 | 457 | 10.60 |
| 51 | 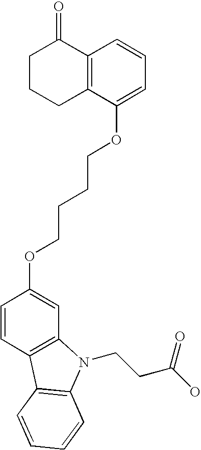 | 472 | 471 | 11.12 |
| 52 | 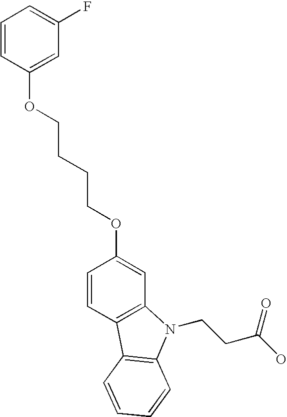 | 421 | 421 | 11.25 |
| 53 | 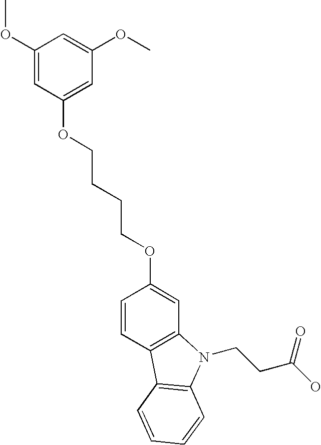 | 464 | 463 | 10.87 |
| 54 | 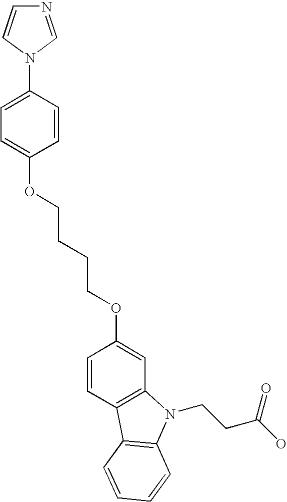 | 470 | 469 | 8.03 |
| 55 | 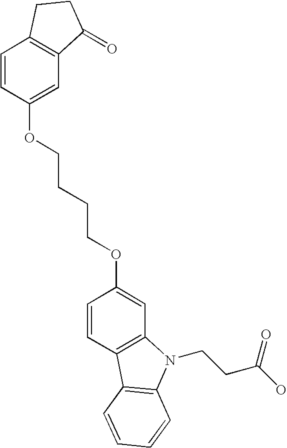 | 458 | 457 | 10.62 |
| 56 | 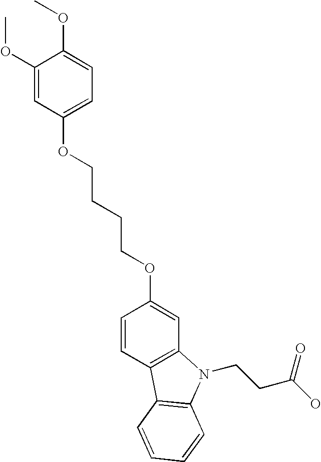 | 464 | 463 | 10.48 |
| 57 | 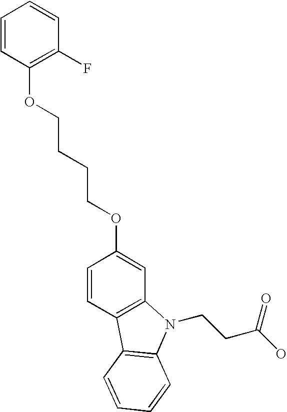 | 421 | 421 | 11.01 |
| 58 | 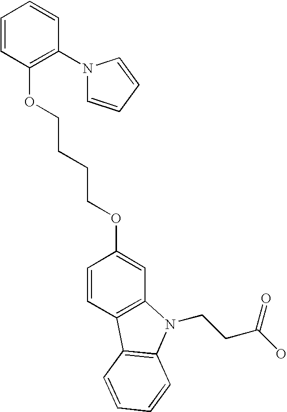 | 469 | 468 | 11.47 |
| 59 | 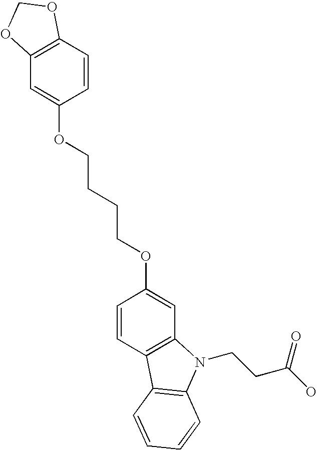 | 447 | 447 | 10.75 |
| 60 | 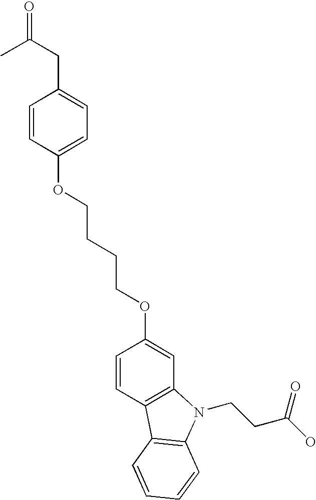 | 460 | 459 | 10.44 |
| 61 | 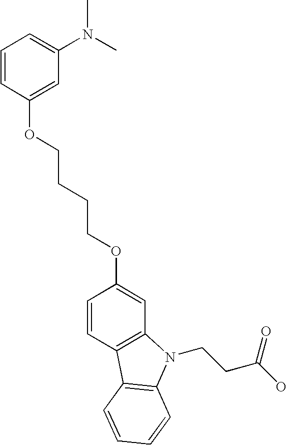 | 447 | 446 | 8.07 |
| 62 | 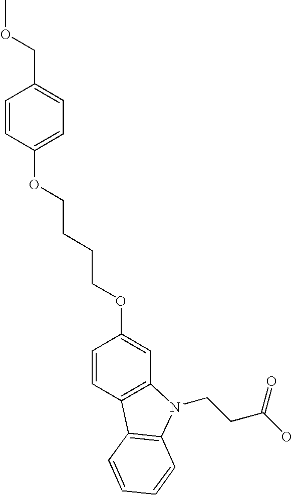 | 448 | 447 | 10.79 |
| 63 | 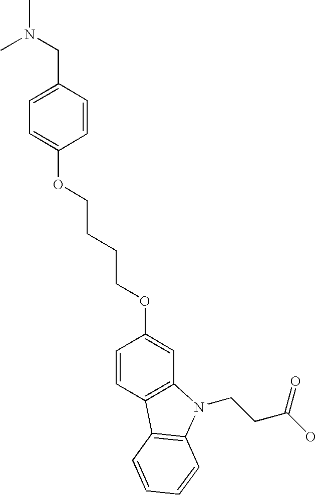 | 461 | 460 | 8.04 |
| 64 | 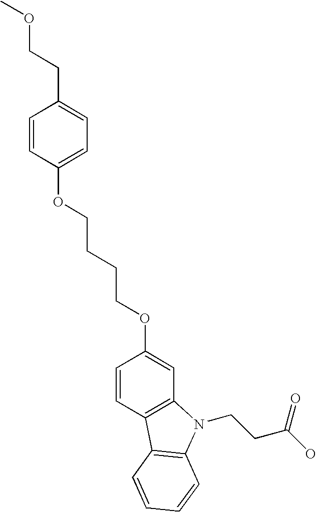 | 462 | 461 | 11.13 |
| 65 | 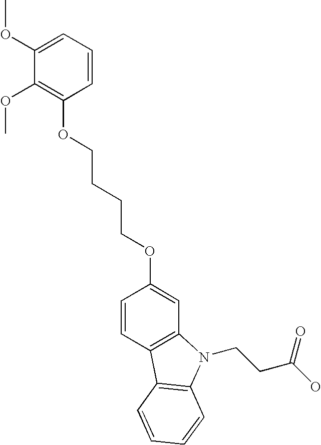 | 464 | 463 | 10.52 |
| 66 | 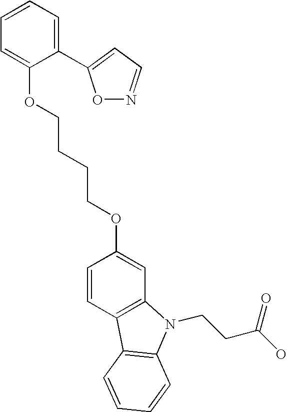 | 471 | 470 | 10.21 |
| 67 | 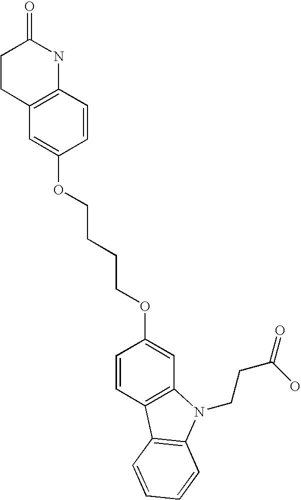 | 473 | 472 | 9.33 |
| 68 | 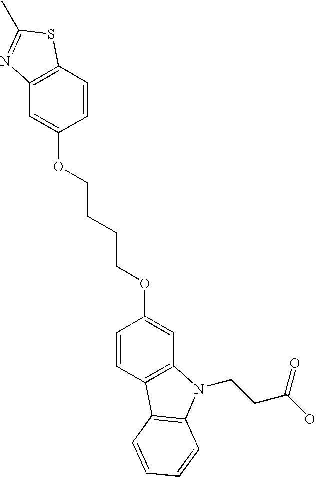 | 475 | 474 | 10.80 |
| 69 | 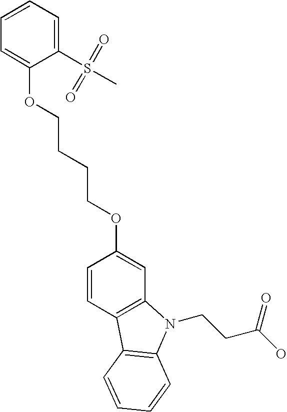 | 482 | 481 | 9.74 |
| 70 | 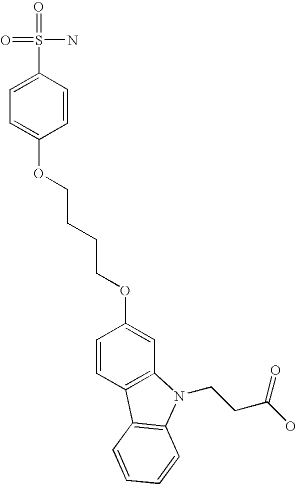 | 483 | 482 | 9.28 |
| 71 | 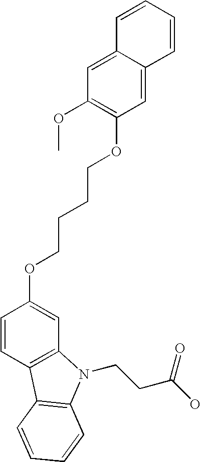 | 484 | 483 | 11.29 |
| 72 | 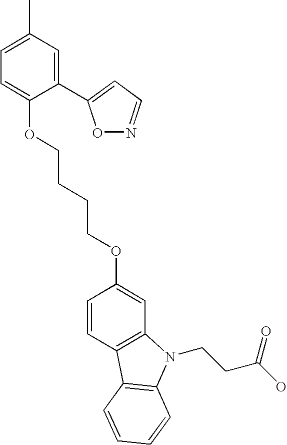 | 485 | 484 | 10.59 |
| 73 | 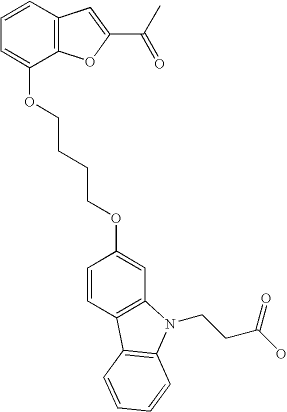 | 486 | 485 | 10.73 |
| 74 | 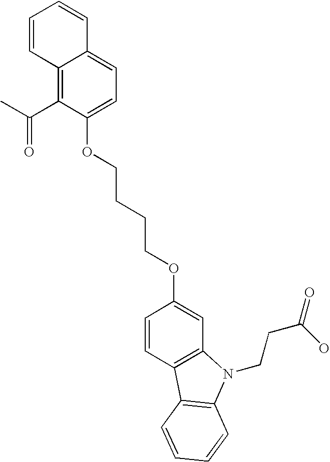 | 496 | 495 | 11.04 |
| 75 | 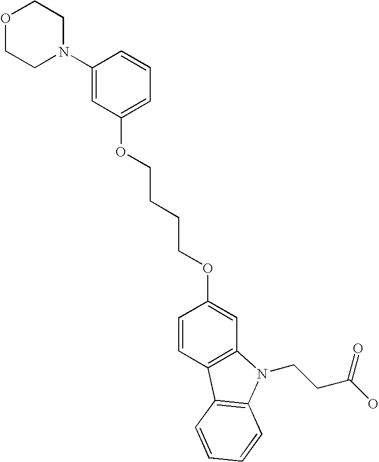 | 489 | 488 | 8.84 |
| 76 | 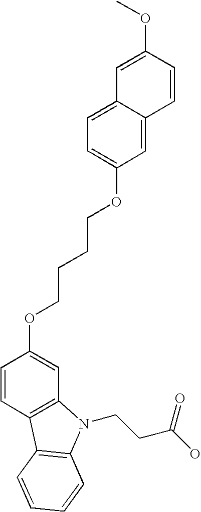 | 484 | 483 | 11.44 |
| 77 | 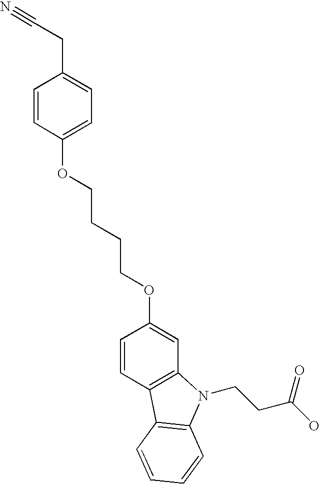 | 443 | 442 | 9.98 |
| 78 | 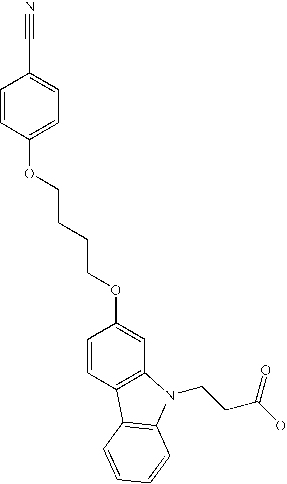 | 428 | 428 | 10.26 |
| 79 | 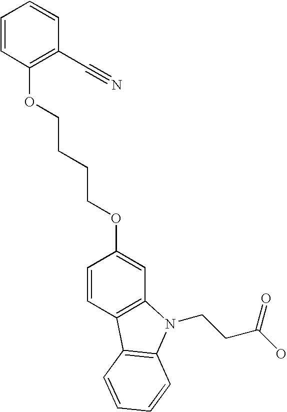 | 428 | 428 | 10.09 |
| 80 | 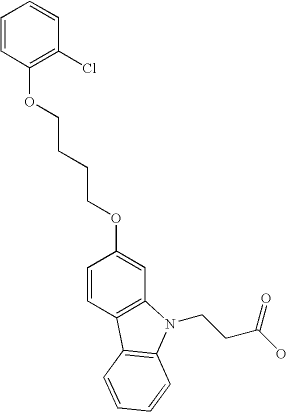 | 438 | 437 | 11.18 |
| 81 | 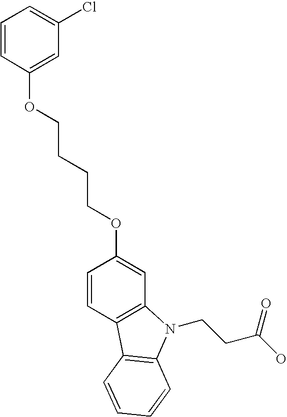 | 438 | 437 | 11.49 |
| 82 | 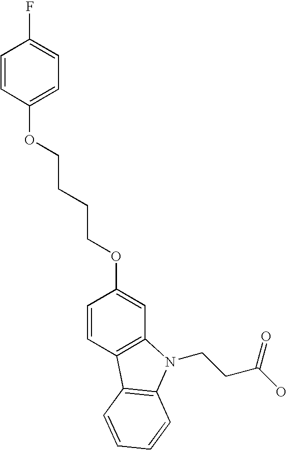 | 421 | 421 | 10.84 |
| 83 | 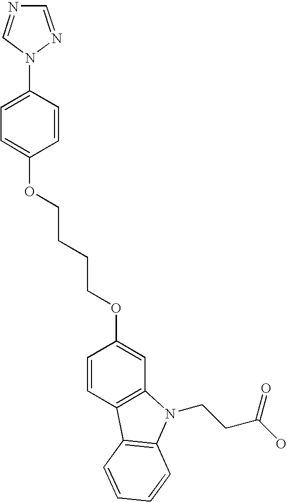 | 471 | 470 | 9.24 |
| 84 | 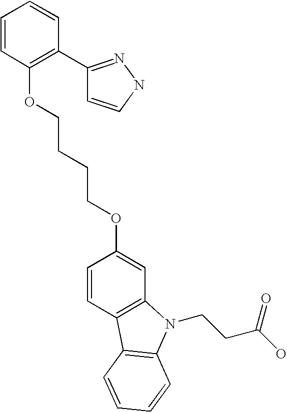 | 470 | 469 | 10.71 |
| 85 | 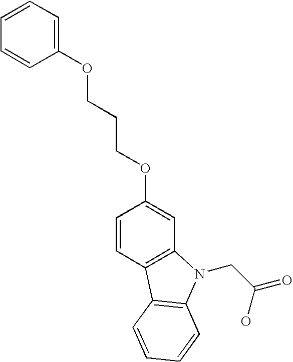 | 375 | 375 | 10.37 |
| 86 | 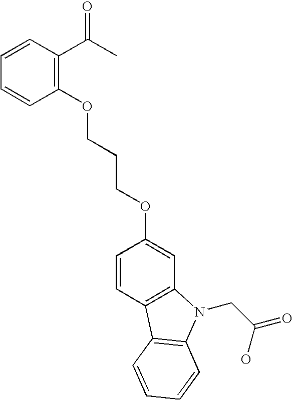 | 417 | 417 | 9.85 |
| 87 | 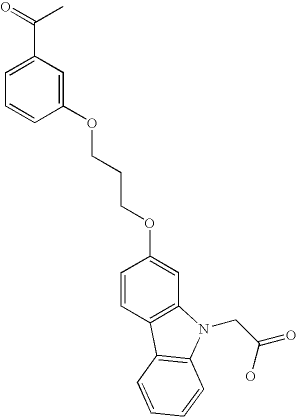 | 417 | 417 | 9.15 |
| 88 | 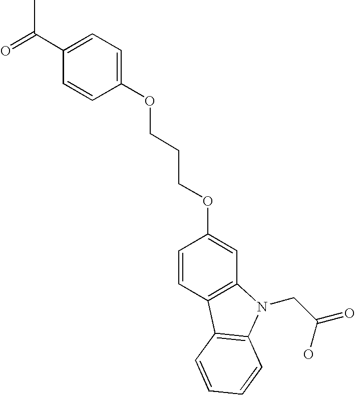 | 417 | 417 | 9.76 |
| 89 | 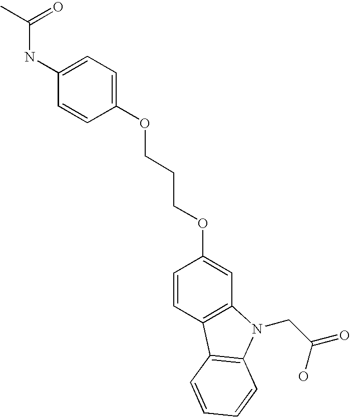 | 432 | 432 | 8.75 |
| 90 | 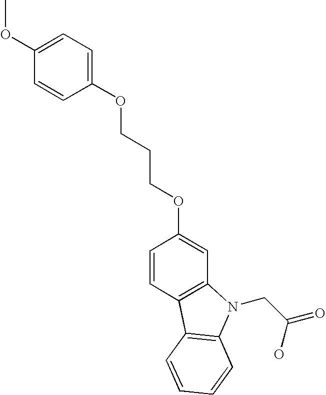 | 405 | 405 | 10.09 |
| 91 | 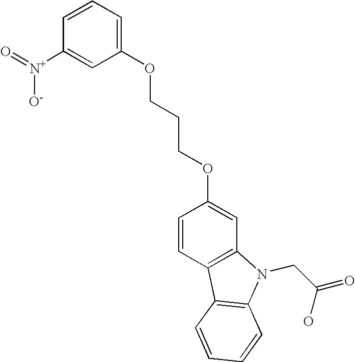 | 420 | 420 | 10.30 |
| 92 | 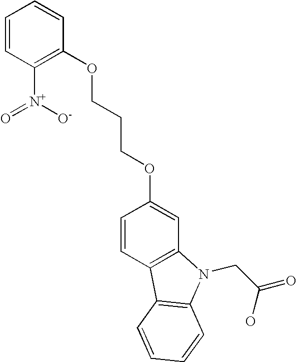 | 420 | 420 | 9.84 |
| 93 | 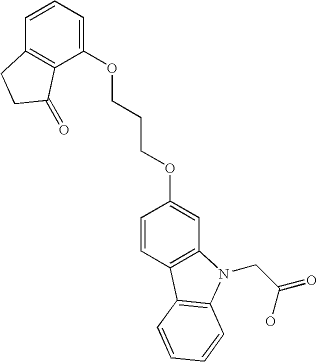 | 429 | 429 | 9.47 |
| 94 | 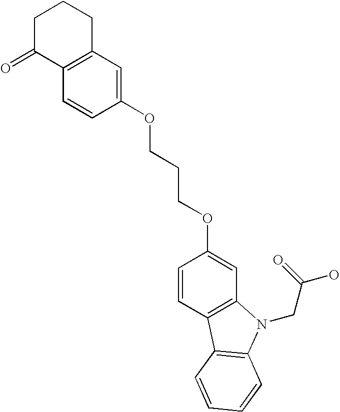 | 444 | 443 | 10.06 |
| 95 | 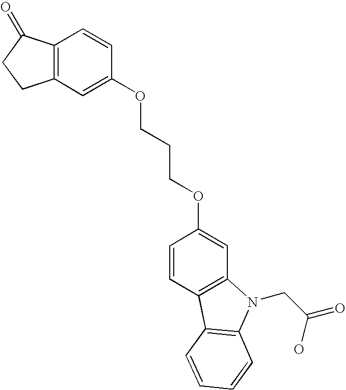 | 429 | 429 | 9.50 |
| 96 | 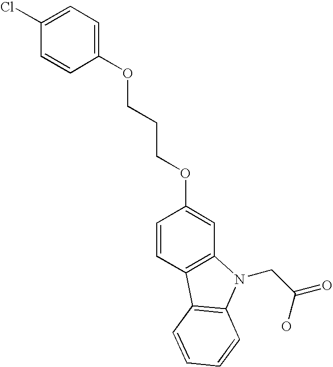 | 410 | 409 | 10.80 |
| 97 | 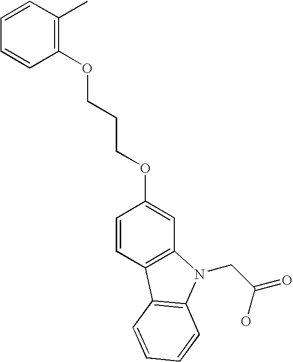 | 389 | 389 | 10.89 |
| 98 | 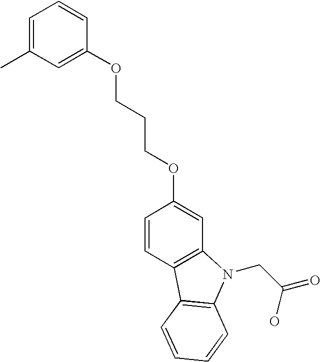 | 389 | 389 | 10.75 |
| 99 | 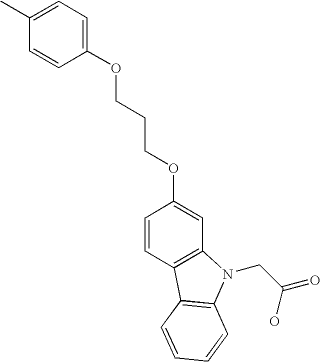 | 389 | 389 | 10.69 |
| 100 | 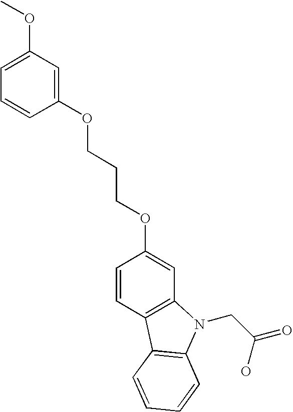 | 405 | 405 | 10.34 |
| 101 | 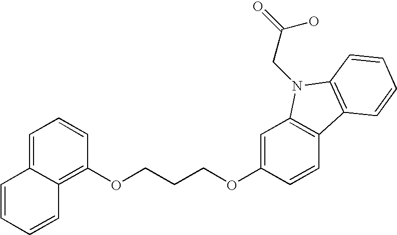 | 425 | 425 | 11.26 |
| 102 | 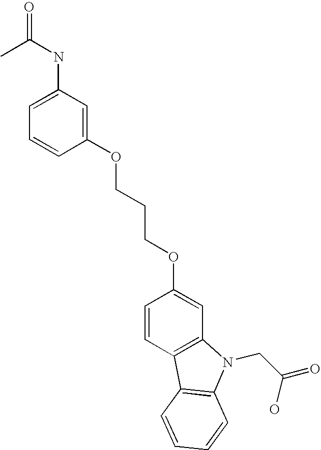 | 432 | 432 | 8.88 |
| 103 | 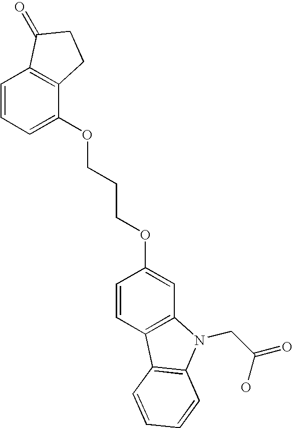 | 429 | 429 | 9.84 |
| 104 | 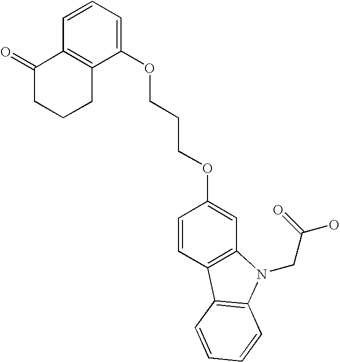 | 444 | 443 | 10.31 |
| 105 | 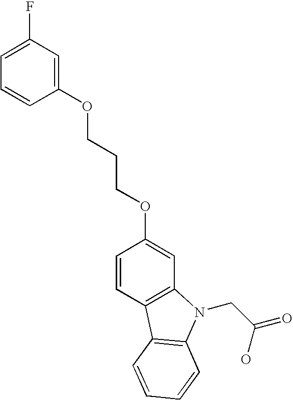 | 393 | 393 | 10.45 |
| 106 | 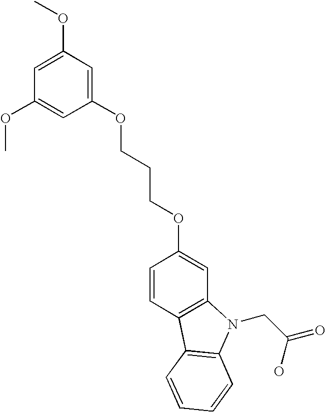 | 435 | 435 | 10.19 |
| 107 | 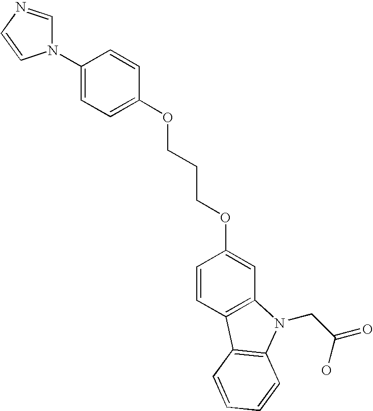 | 441 | 441 | 7.50 |
| 108 | 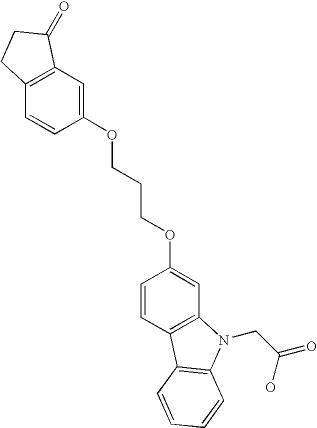 | 429 | 429 | 9.82 |
| 109 | 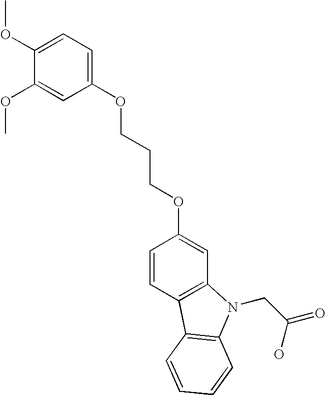 | 435 | 435 | 9.72 |
| 110 | 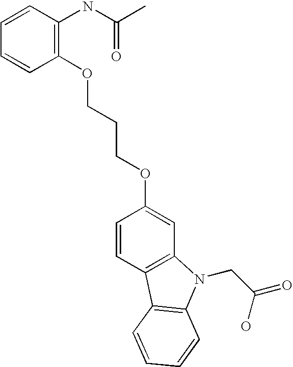 | 432 | 432 | 9.15 |
| 111 | 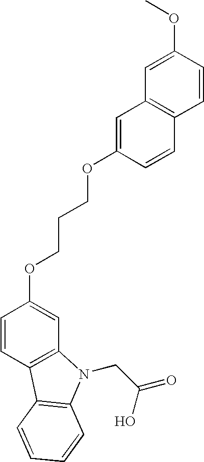 | 456 | 455 | 11.07 |
| 112 | 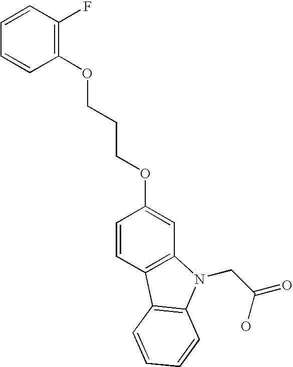 | 393 | 393 | 10.31 |
| 113 | 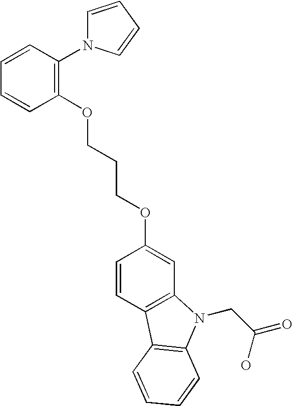 | 441 | 440 | 10.78 |
| 114 | 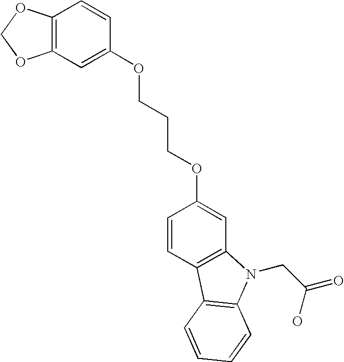 | 419 | 419 | 10.05 |
| 115 | 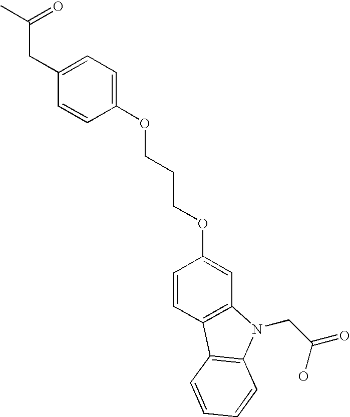 | 431 | 431 | 9.77 |
| 116 | 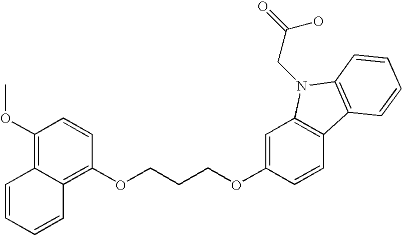 | 456 | 455 | 11.25 |
| 117 | 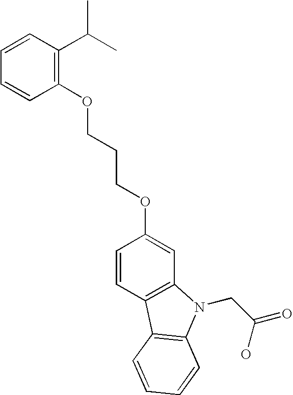 | 418 | 417 | 11.54 |
| 118 | 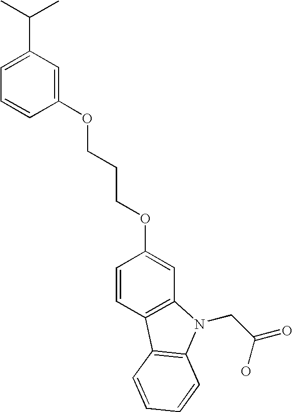 | 418 | 417 | 11.42 |
| 119 | 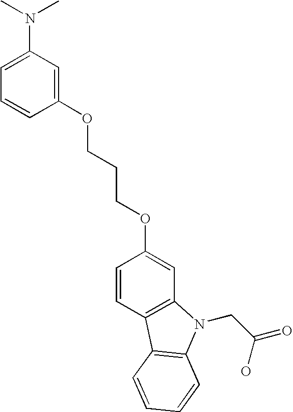 | 418 | 418 | 7.55 |
| 120 | 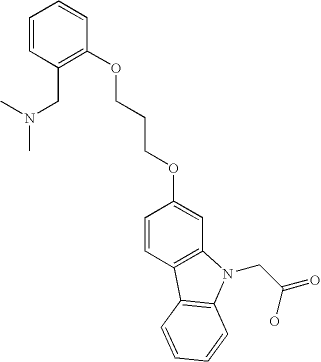 | 433 | 432 | 7.62 |
| 121 | 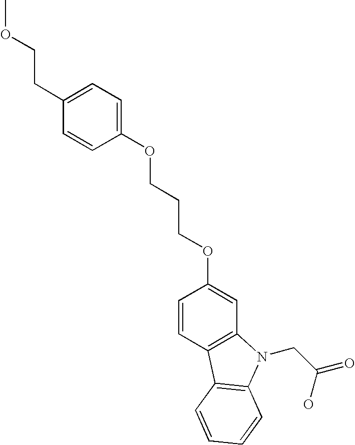 | 434 | 433 | 10.33 |
| 122 | 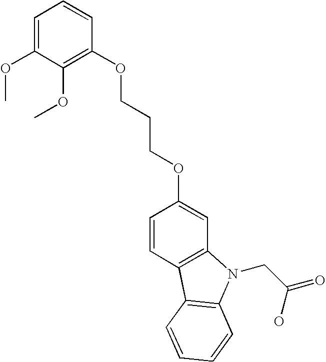 | 435 | 435 | 9.82 |
| 123 | 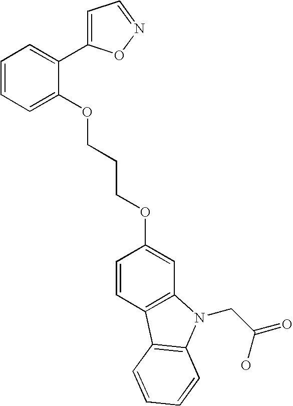 | 442 | 442 | 9.65 |
| 124 | 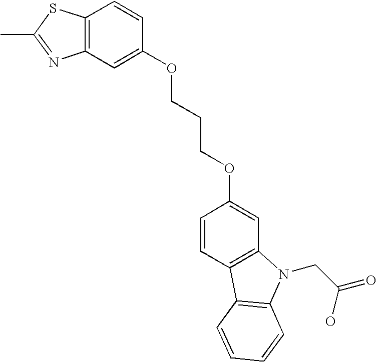 | 447 | 446 | 10.0 |
| 125 | 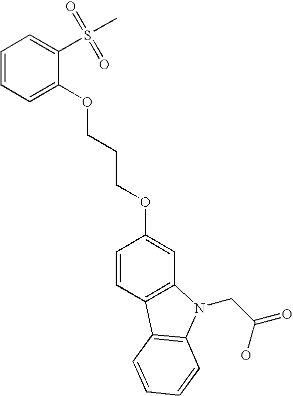 | 454 | 453 | 9.17 |
| 126 | 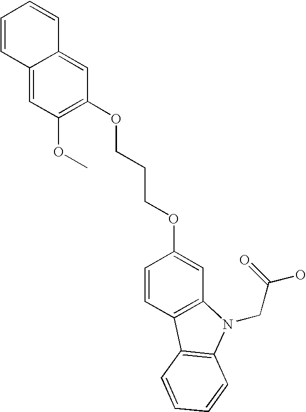 | 456 | 455 | 10.65 |
| 127 | 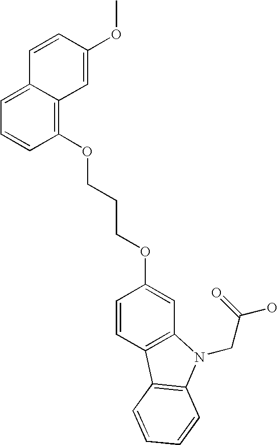 | 456 | 455 | 11.24 |
| 128 | 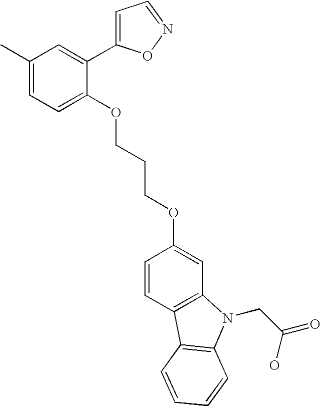 | 457 | 456 | 10.02 |
| 129 | 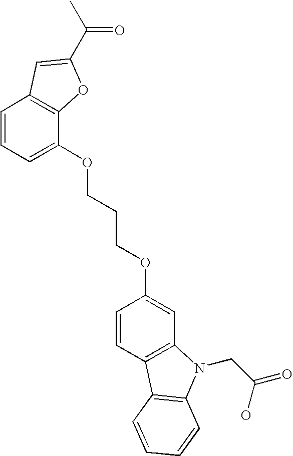 | 457 | 457 | 10.05 |
| 130 | 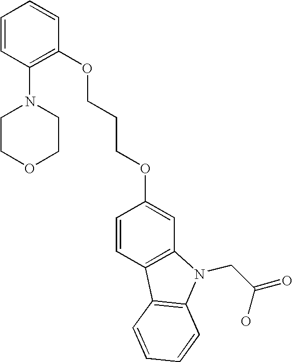 | 461 | 460 | 7.85 |
| 131 | 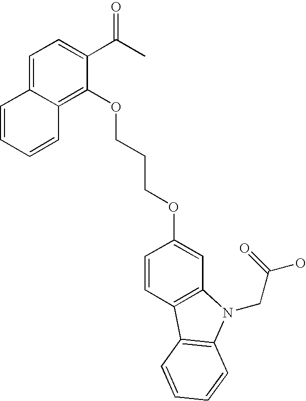 | 468 | 467 | 10.75 |
| 132 | 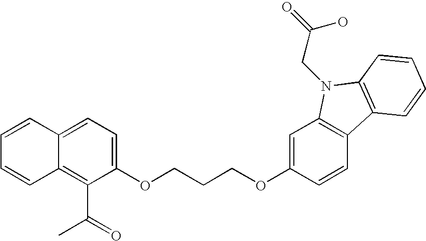 | 468 | 467 | 10.70 |
| 133 | 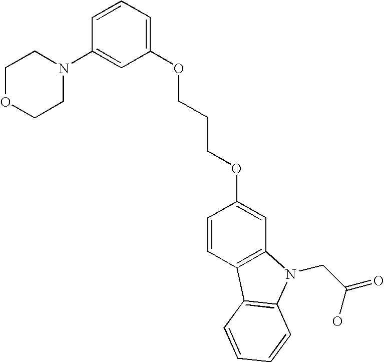 | 461 | 460 | 8.95 |
| 134 | 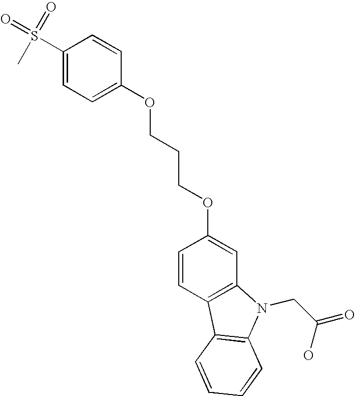 | 454 | 453 | 9.19 |
| 135 | 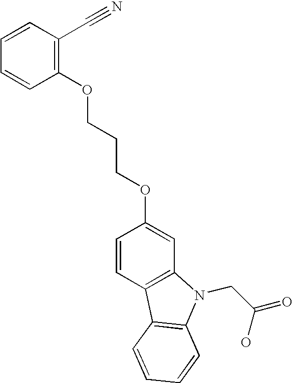 | 400 | 400 | 9.89 |
| 136 | 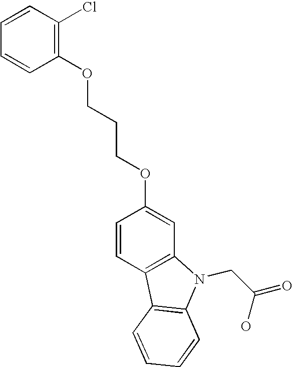 | 410 | 409 | 10.72 |
| 137 | 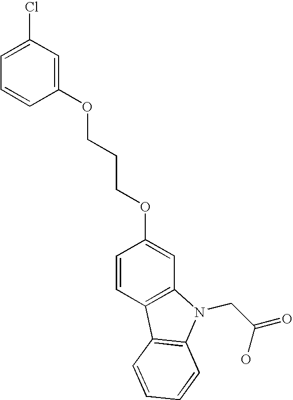 | 410 | 409 | 10.95 |
| 138 | 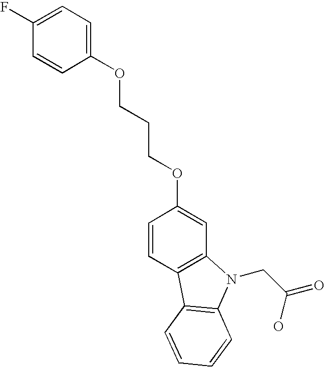 | 393 | 393 | 10.37 |
| 139 | 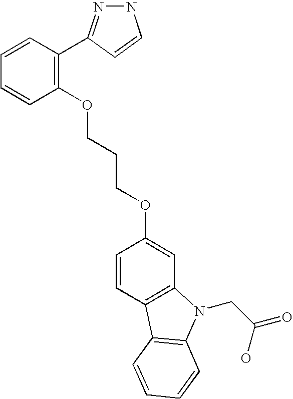 | 441 | 441 | 9.04 |
| 140 | 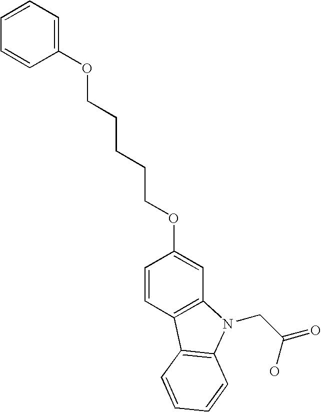 | 403 | 403 | 11.00 |
| 141 | 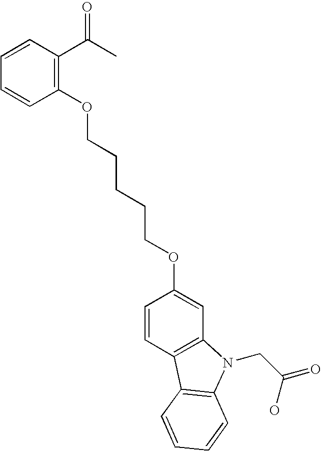 | 446 | 445 | 10.62 |
| 142 | 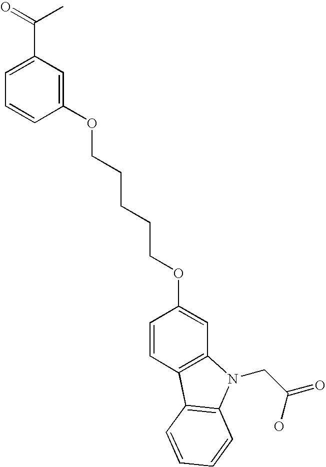 | 446 | 445 | 10.62 |
| 143 | 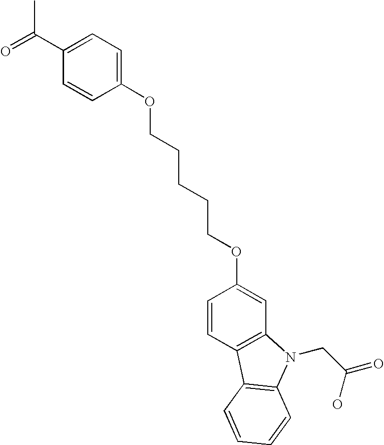 | 446 | 445 | 10.47 |
| 144 | 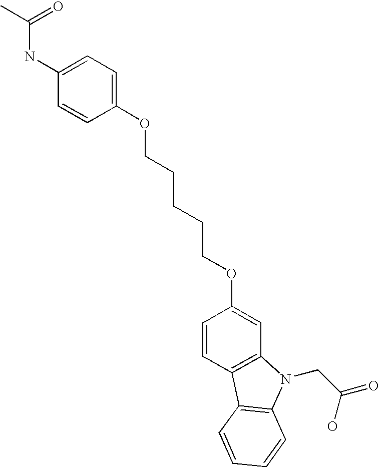 | 461 | 460 | 9.35 |
| 145 | 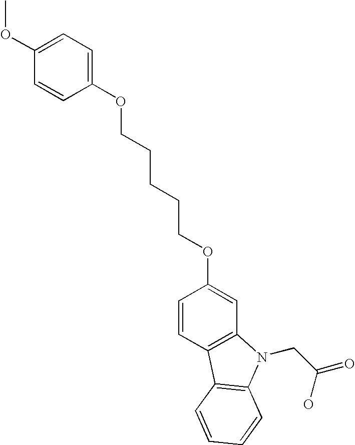 | 434 | 433 | 10.79 |
| 146 | 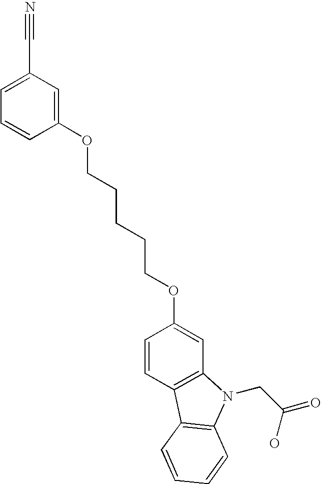 | 428 | 428 | 10.74 |
| 147 | 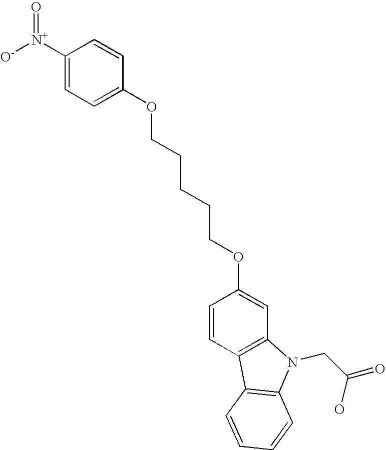 | 448 | 448 | 10.90 |
| 148 | 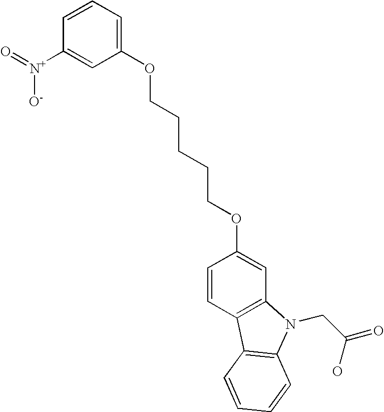 | 448 | 448 | 10.93 |
| 149 | 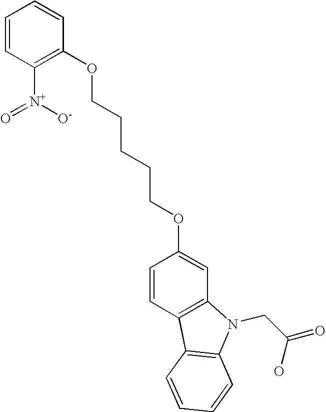 | 448 | 448 | 10.49 |
| 150 | 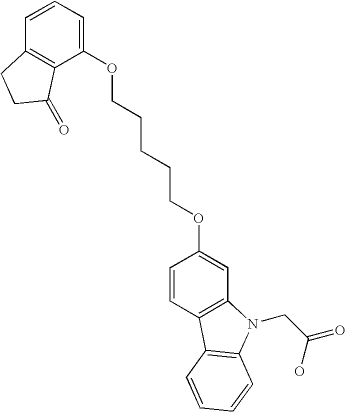 | 458 | 457 | 10.12 |
| 151 | 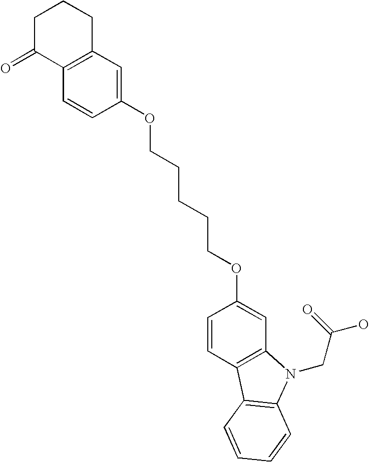 | 472 | 471 | 10.79 |
| 152 | 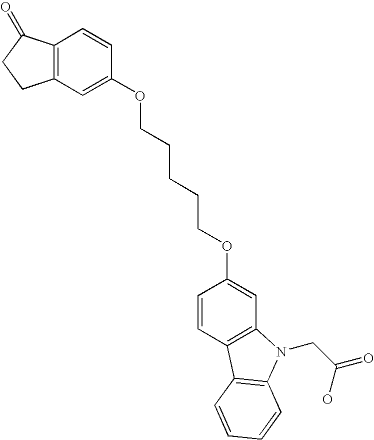 | 458 | 457 | 10.22 |
| 153 | 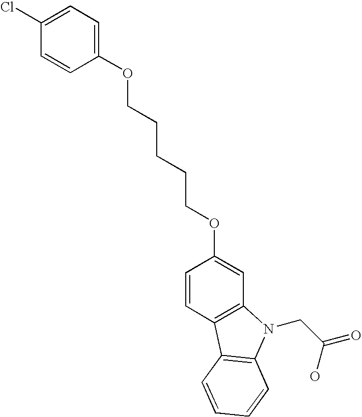 | 438 | 437 | 11.50 |
| 154 | 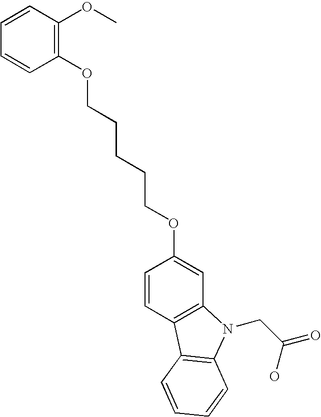 | 434 | 433 | 10.64 |
| 155 | 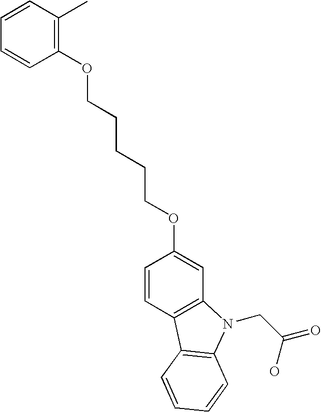 | 418 | 417 | 11.59 |
| 156 | 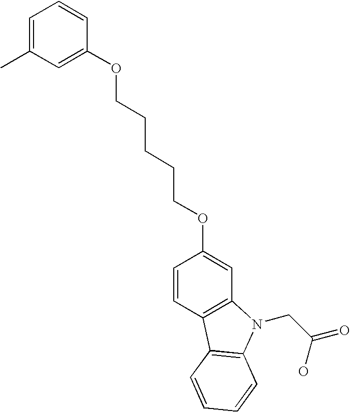 | 418 | 417 | 11.35 |
| 157 | 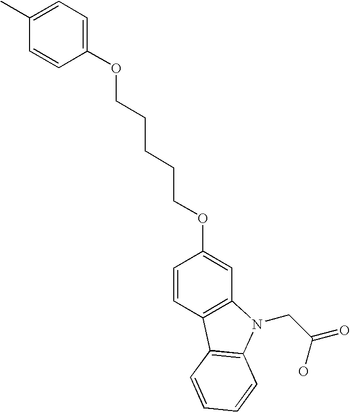 | 418 | 417 | 11.35 |
| 158 | 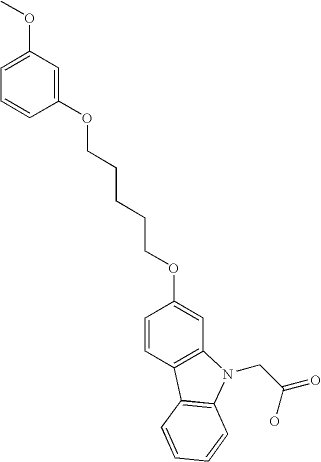 | 434 | 433 | 10.99 |
| 159 | 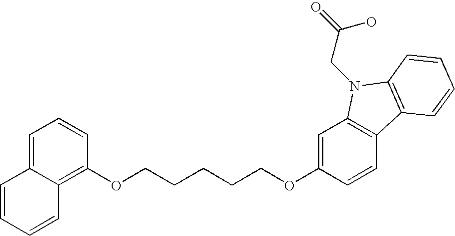 | 454 | 453 | 11.92 |
| 160 | 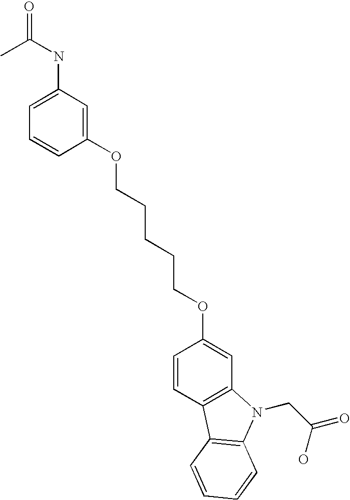 | 461 | 460 | 9.57 |
| 161 | 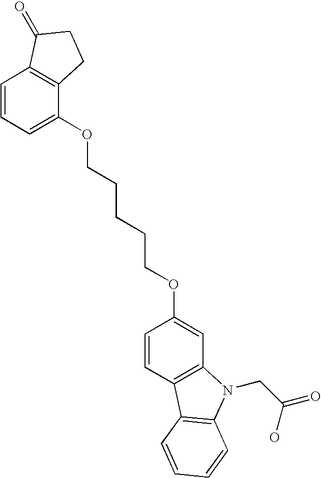 | 458 | 457 | 10.57 |
| 162 | 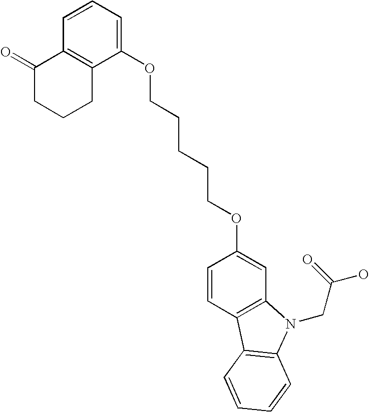 | 472 | 471 | 11.07 |
| 163 | 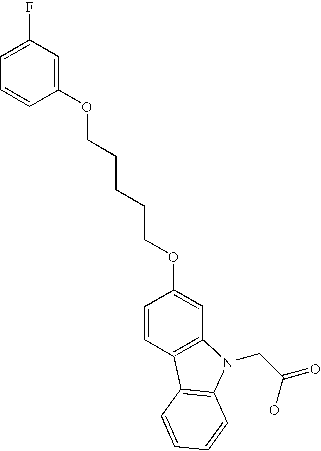 | 421 | 421 | 11.08 |
| 164 | 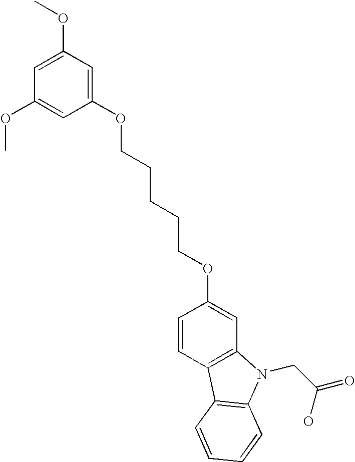 | 464 | 463 | 10.82 |
| 165 | 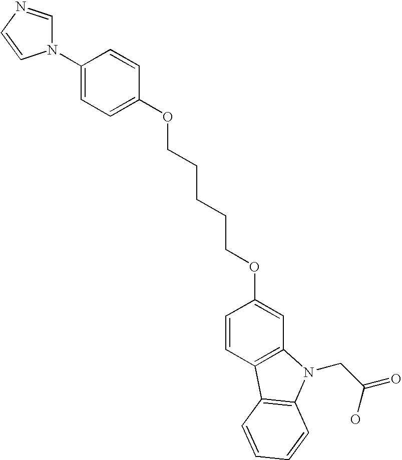 | 470 | 469 | 7.90 |
| 166 | 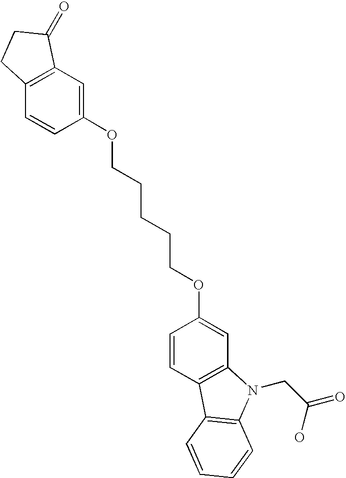 | 458 | 457 | 10.56 |
| 167 | 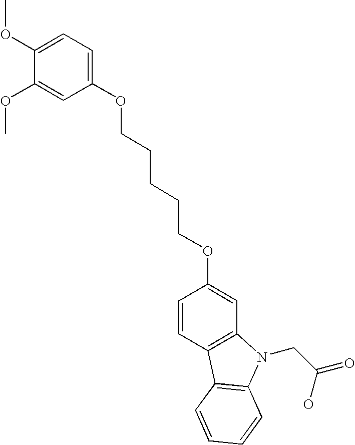 | 464 | 463 | 10.35 |
| 168 | 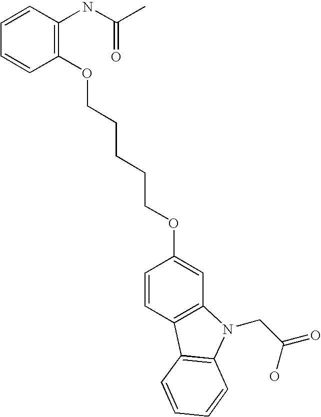 | 461 | 460 | 9.82 |
| 169 | 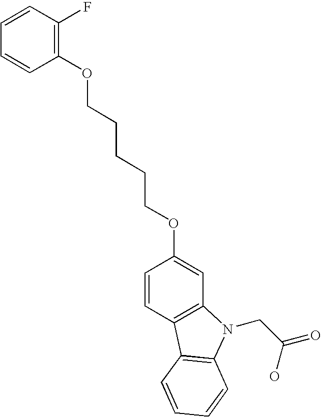 | 421 | 421 | 10.96 |
| 170 | 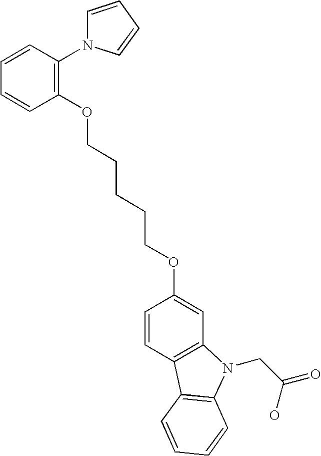 | 469 | 468 | 11.33 |
| 171 | 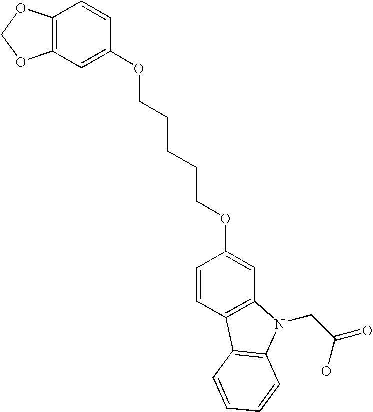 | 447 | 477 | 10.70 |
| 172 | 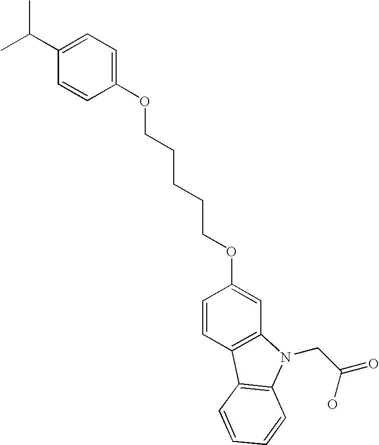 | 446 | 445 | 12.05 |
| 173 | 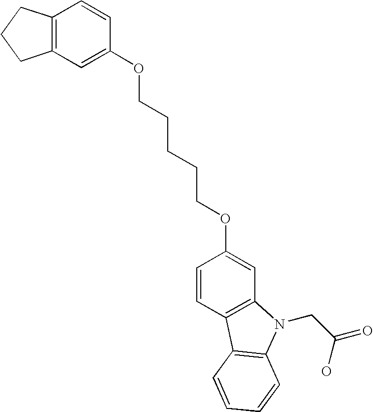 | 444 | 443 | 11.91 |
| 174 | 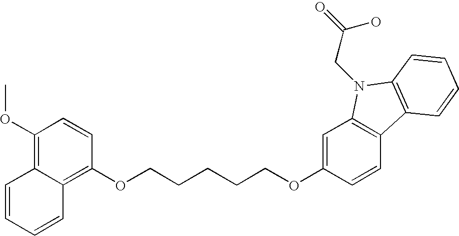 | 484 | 483 | 11.90 |
| 175 | 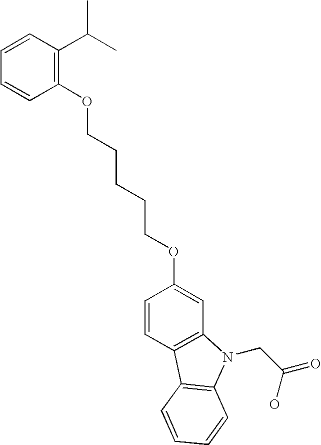 | 446 | 445 | 12.18 |
| 176 | 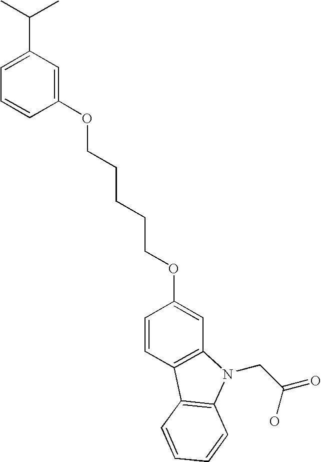 | 446 | 445 | 11.99 |
| 177 | 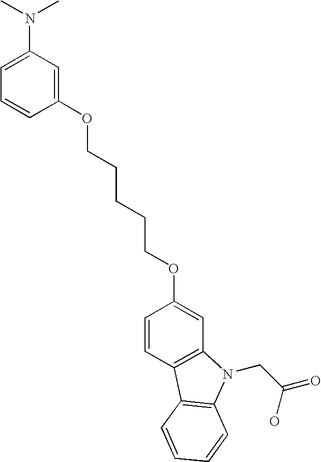 | 447 | 446 | 8.08 |
| 178 | 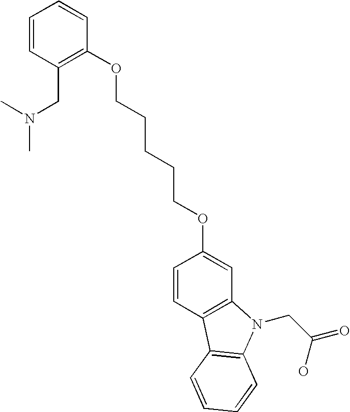 | 461 | 460 | 8.00 |
| 179 | 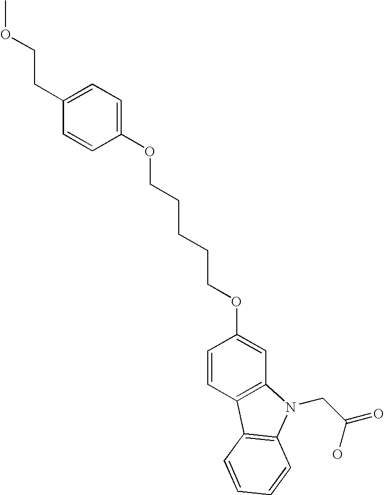 | 462 | 461 | 10.98 |
| 180 | 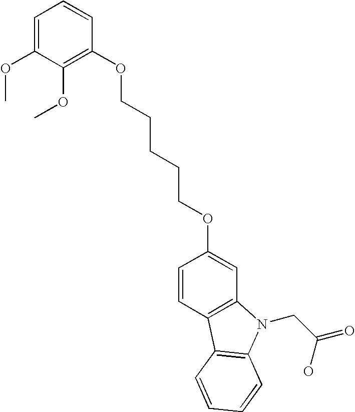 | 464 | 463 | 10.50 |
| 181 | 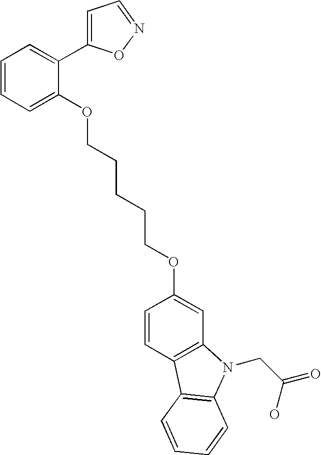 | 471 | 470 | 10.22 |
| 182 | 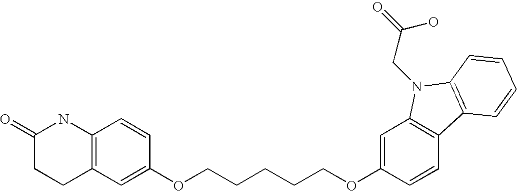 | 473 | 472 | 9.32 |
| 183 | 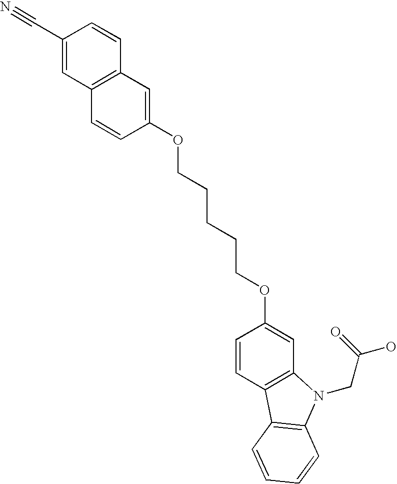 | 479 | 478 | 11.39 |
| 184 | 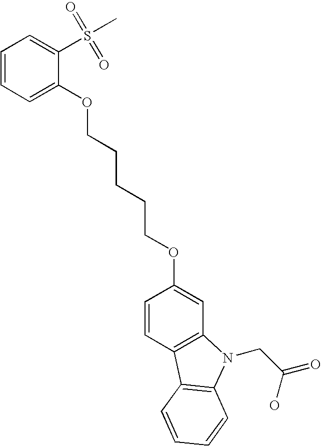 | 482 | 481 | 9.72 |
| 185 | 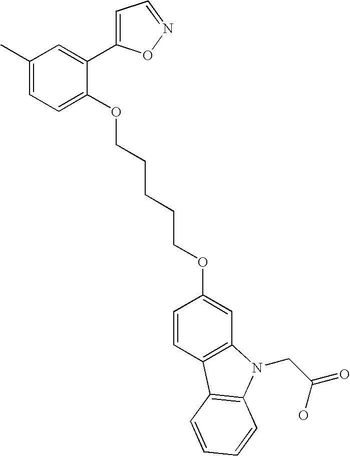 | 485 | 484 | 10.59 |
| 186 | 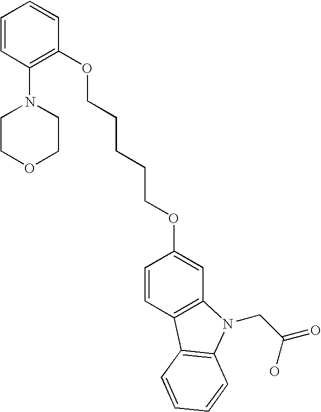 | 489 | 488 | 8.37 |
| 187 | 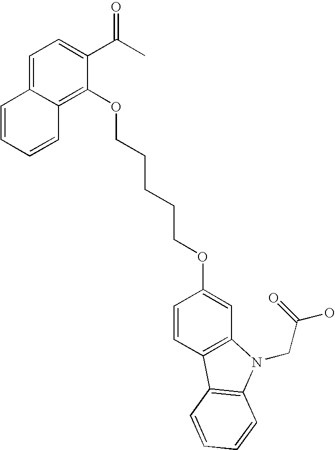 | 496 | 495 | 11.32 |
| 188 | 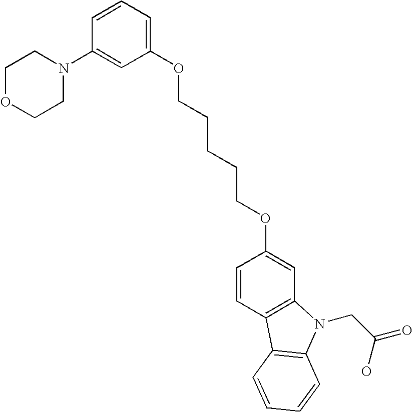 | 489 | 488 | 9.66 |
| 189 | 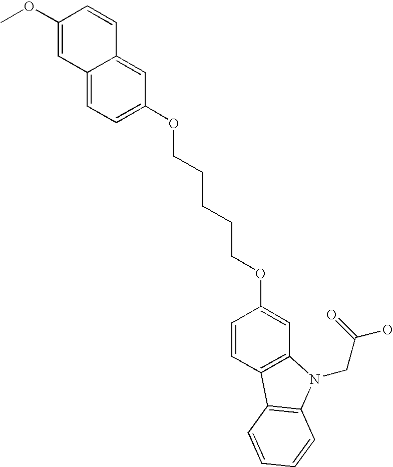 | 484 | 483 | 11.52 |
| 190 | 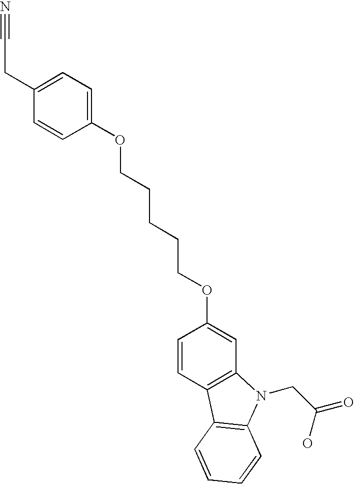 | 443 | 442 | 10.39 |
| 191 | 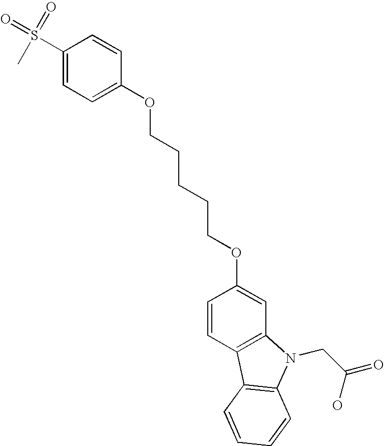 | 482 | 481 | 9.77 |
| 192 | 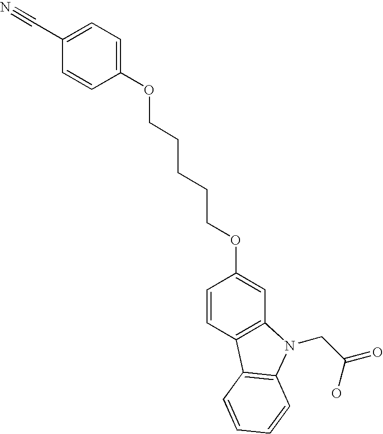 | 428 | 428 | 10.50 |
| 193 | 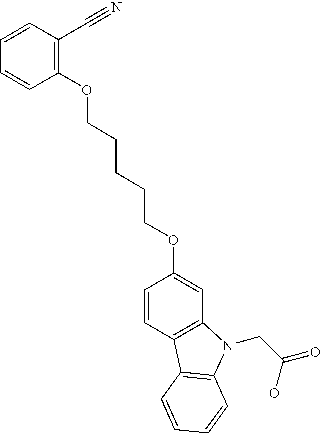 | 428 | 428 | 10.49 |
| 194 | 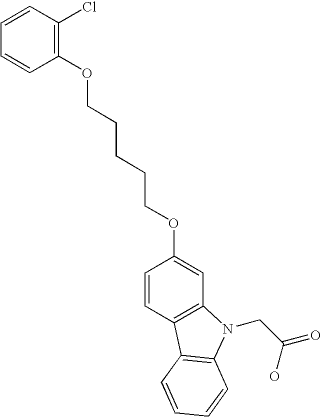 | 438 | 437 | 11.36 |
| 195 | 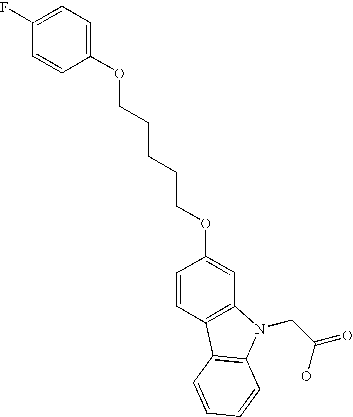 | 421 | 421 | 10.95 |
| 196 | 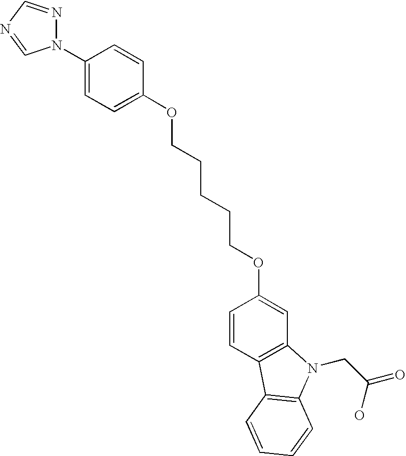 | 471 | 470 | 9.89 |
| 197 | 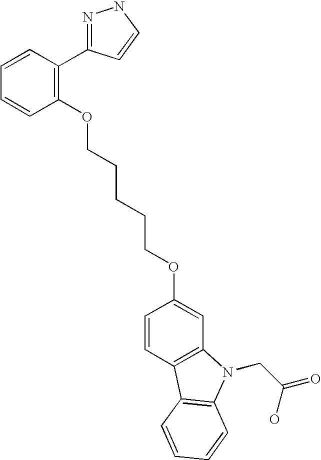 | 470 | 469 | 9.69 |

### BIOLOGICAL EXAMPLES:

1. Detection of the Antagonism of the Human Prostaglandin E₂ (Subtype EP₂) Receptor Signal

1.1 Principle of Detection

The binding of PGE2 to the EP2 subtype of the human PGE2 receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nM). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

1.2. Detection Method

1.2.1 Antagonism Assay (Data for Each Well of a 384-Well Plate):

The substance solutions (0.75 μl) introduced into an assay plate and 30% DMSO are dissolved in 16 μl of a KRSB+IBMX stimulation solution (1×Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After preincubation at room temperature (RT) for 30 minutes, 5 μl of a 4×PGE₂ solution (11 nM) are added, and incubation is carried out in the presence of the agonist at RT for a further 60 min (volume: ˜20 μl) before the reaction is then stopped by adding 5 μl of lysis buffer and incubated at RT for a further 20 min (volume: ˜25 μl). The cell lysate is then transferred into a measuring plate and measured in accordance with the manufacturer's information (cyclic AMP kit Cisbio International # 62AMPPEC).

1.2.2 Agonism Assay (Data for Each Well of a 384-Well Plate):

The substance solutions (0.75 μl) introduced into an assay plate and 30% DMSO are dissolved in 16 μl of a KRSB+IBMX stimulation solution (1×Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After incubation at room temperature (RT; volume: ˜15 μl) for 60 minutes, the reaction is then stopped by adding 5 μl of lysis buffer and incubated at RT for a further 20 min (volume: ˜20 μl). The cell lysate is then transferred into a measuring plate and measured in accordance with the manufacturer's information (cyclic AMP kit Cisbio International # 62AMPPEC).

2. The EP2 Subtype of the PGE2 Receptor and the Preovulatory Cumulus Expansion

2.1. Background:

In the preovulatory antral follicle, the oocyte is surrounded by cumulus cells which form a dense ring of cells around the oocyte. After the LH peak (lutenising hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140 (2):307-317). This cumulus expansion is an important component of the ovulatory process and of the subsequent possibility of fertilisation.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 August 31; 96 (18):10501-6.) show a markedly reduced cumulus expansion and severe subfertility, demonstrating the importance of the prostanoid EP₂ receptor for this process.

2.2 Cumulus Expansion Assay in Vitro

Folliculogenesis is induced in immature female mice (strain: CD1 (ICR) from Charles River) at an age of 14-18 days by a single dose (intraperitoneal) of 10 I.U. of PMSG (Pregnant Mare Serum Gonadotropin; Sigma G-4877, Lot 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E₂ (PGE₂) (1 μM), vehicle control (ethanol) or test substances for 20-24 hours. Medium: alpha-MEM medium with 0.1 mM IBMX, pyruvates (0.23 mM) glutamines (2 mM), pen/strep 100 IU/ml pen. and 100 μg/ml strep. and HSA (8 mg/ml). Cumulus expansion is then established through the division into four stages (according to Vanderhyden et al. Dev Biol. 1990 August; 140 (2):307-317).

TABLE 1
Example of the biological activity of the
inventive compounds (measured by means of the cAMP
antagonism test):

| Substance according | Agonism | Antagonism |
|---|---|---|
| to example | [ED₅₀, μm] | [IC₅₀, μm] |
| VI/3 | >19 | 6.4 |
| XIII/12 | >19 | 6.3 |
| XIII/22 | >19 | 1.2 |
| XIII/67 | >19 | 3.2 |
| XIII/85 | >19 | 6.7 |
| XIII/140 | >19 | 6.7 |
| AH6809 | >19 | >19 |
| XII/5 | 6.3 | >19 |
| VI/4 | 6.3 | >19 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102005062741.2, filed Dec. 22, 2005, and U.S. Provisional Application Ser. No. 60/754,186, filed Dec. 28, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the general formula D 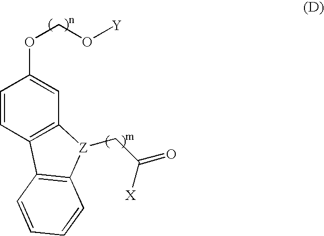
where
Y is a 5-12-membered, mono- or bicyclic aryl or heteroaryl radical which may optionally be mono- or polysubstituted by
a C₁-C₆-alkyl which may be straight or branched, saturated or unsaturated, and may optionally be mono- or polysubstituted by R¹ where R¹ is the —O—C₁-C₆-alkyl group, cyano, —N(C₁-C₆-alkyl)₂, —NH—C₁-C₆-alkyl, —C₄-C₈-cycloamine, —NH₂, —CO—CH₃—, —SO₂—NH₂, —C₁-C₆-alkyl, —SO₂—NH—CO—C₁-C₆-alkyl, —SO₂—NH—C₁-C₆-alkyl, —SO₂—C₁-C₆-alkyl, —NH—CO—C₁-C₆-alkyl, NO₂, —OH, —COOH or halogen,
R¹ where R¹ is as defined above,
a saturated or unsaturated 4-8-membered heterocycle which optionally bears 1-3 nitrogen or oxygen atoms and is optionally substituted by —OCH₃, —COCH₃, —C₁-C₆-alkyl, —C₁-C₂-alkyl-O—CH₃ or a keto group,
an —SO₂—NH₂, —SO₂-CH₃, —NH—CO—CH₃ group, NO₂, —OH, —COOH or halogen,
a fused 5-7-membered carbocycle optionally substituted by a keto group,
a 6-12-membered, mono- or bicyclic heterocycle which may be saturated or unsaturated, is interrupted once or more than once by nitrogen, oxygen or sulfur, and is optionally mono- or polysubstituted by
R¹ , where R¹ is as defined above,
an oxygen or a keto group,
Z is a carbon or nitrogen radical,
X is an —OH, —NH₂ group, an —O—C₁-C₆-alkyl, —N(C₁-C₆-alkyl)₂, —NH—C₁-C₆-alkyl, C₄-C₈-cycloamine, —NH—SO₂—C₁-C₆-alkyl or a saturated or unsaturated —C₃-C₈-cycloalkyl radical which may optionally be substituted,
n is 1-7,
m is 1-4 and their salts with physiologically acceptable bases and their cyclodextrin clathrates.

2. A compound as claimed in claim 1 where
Y is a 5-12-membered, mono- or bicyclic aryl or heteroaryl radical which may optionally be mono- or polysubstituted by
a C₁-C₆-alkyl which may be straight or branched, saturated or unsaturated, and may optionally be mono- or polysubstituted by R², where R² is the —O—C₁-C₃-alkyl group, cyano, —N(C₁-C₃-alkyl)₂, NH—C₁-C₃-alkyl, —NH₂, —CO—CH₃-, —C₁-C₃-alkyl, —SO₂—NH₂, —SO₂—NH—CO—C₁-C₃-alkyl, SO₂—NHC₁-C₃-alkyl, —SO₂—C₁-C₃-alkyl, —NH—CO—C₁-C₃-alkyl, NO₂, —OH, —COOH, —C₄-C₆-cycloamine or halogen,
R², where R² is as defined above,
a saturated or unsaturated 5-7-membered heterocycle which optionally bears 1-3 nitrogen or oxygen atoms and is optionally substituted by —OCH₃, —C₁-C₃-alkyl, —C₁-C₂-alkyl-OCH₃ or a keto group,
an —SO₂—NH₂, —SO₂-CH₃, —NH—CO—CH₃ group, —NO₂, —OH, —COOH or halogen,
a fused 5-7-membered carbocycle optionally substituted by a keto group,
a 6-12-membered, mono- or bicyclic heterocycle which may be saturated or unsaturated, is interrupted once or more than once by nitrogen, oxygen or sulfur, and is optionally mono- or polysubstituted by an —OCH₃, —CO—CH₃ group, —C₁-C₃-alkyl, cyano, oxygen or a keto group,
Z is a carbon or nitrogen radical,
X is an OH or NH₂ group,
an —O—C₁-C₆-alkyl, —N(C₁-C₆-alkyl)₂, —NH—C₁-C₆-alkyl, —C₄-C₈-cycloamine, —NH—SO₂—C₁-C₆-alkyl or a saturated or unsaturated —C₃-C₈-cycloalkyl radical which may optionally be substituted,
n is 2-5,
m is 1-3.

3. A compound as claimed in claim 1 where
Y is a 6-12-membered mono- or bicyclic aryl radical which may optionally be mono- or polysubstituted and which is optionally mono- or polysubstituted by
a C₁-C₆-alkyl which may be straight or branched, saturated or unsaturated, and may optionally be mono- or polysubstituted by R³, where R³ is the —OCH₃ group, —CO—CH₃, cyano, —NH₂, —N(CH₃)₂, —NHCH₃, —SO₂—NH₂, —SO₂—NH—CO—CH₃, —SO₂—NH—CH₃, —SO₂—CH₃, —NH—CO—CH₃, —NO₂, —OH, —COOH or halogen,
R³, where R³ is as defined above,
an —SO₂—NH₂, —SO₂—CH₃, —NH—CO—CH₃ group, NO₂, —OH, —COOH or halogen,
or the group 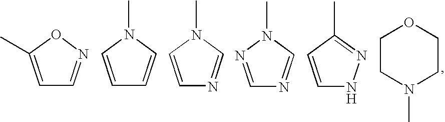
represent the group 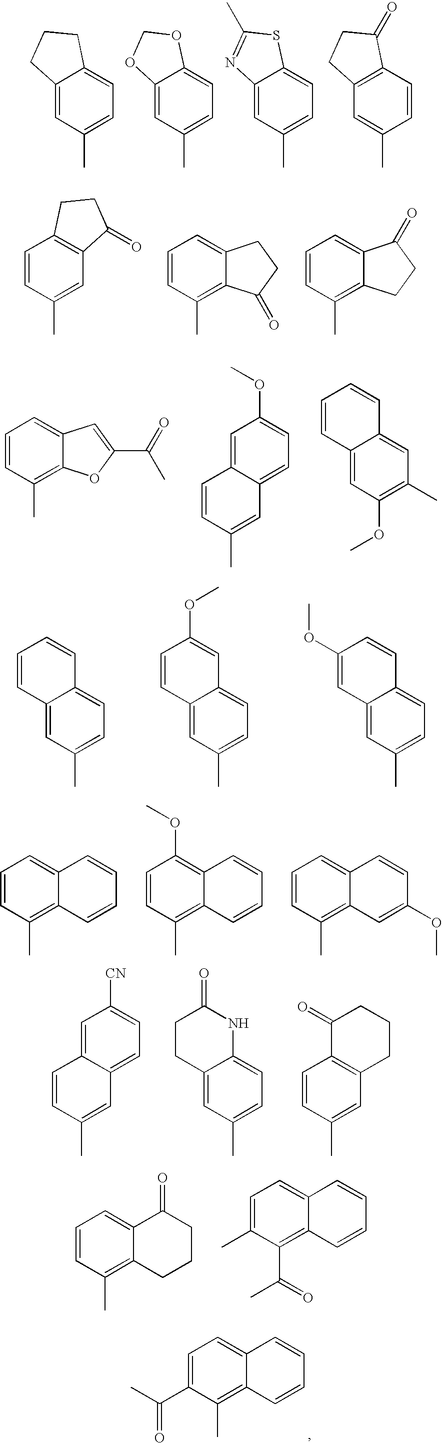
z is a carbon or a nitrogen radical,
x is a hydroxyl group, —N(CH₃)₂, —NH—CH₃, —NHSO₂—CH₃,
n is 3-5,
m is 1-2.

4. A compound as claimed in claim 1 selected from a group which comprises the following compounds:
(R/S)[2-(4-m-tolyloxybutoxy)-9H-fluoren-9-yl]acetic acid
(R/S) {2-[4-(3-hydroxy-5-methoxyphenoxy)butoxy]-9H-fluoren-9-yl}acetic acid
(R/S) {2-[4-(3-hydroxyphenoxy)butoxy]-9H-fluoren-9-yl}acetic acid
(R/S) {2-[4-(3-acetylaminophenoxy)butoxy]-9H-fluoren-9-yl}acetic acid
ethyl {2-[4-(3-hydroxyphenoxy)butoxy]carbazol-9-yl}acetate
ethyl [2-(4-m-tolyloxybutoxy)carbazol-9-yl]acetate
ethyl {2-[4-(3-acetylaminophenoxy)butoxy]carbazol-9-yl}acetate
ethyl {2-[4-(3-hydroxy-5-methoxyphenoxy)butoxy]carbazol-9-yl}acetate
methyl 3-[4-(9-ethoxycarbonylmethyl-9H-carbazol-2-yloxy)butoxy]benzoate
ethyl 3-{2-[4-(3-hydroxyphenoxy)butoxy]carbazol-9-yl}propionate
ethyl 3-[2-(4-m-tolyloxybutoxy)carbazol-9-yl]propionate
ethyl 3-{2-[4-(3-acetylaminophenoxy)butoxy]carbazol-9-yl}propionate
ethyl 3-{2-[4-(3-hydroxy-5-methoxyphenoxy)butoxy]carbazol-9-yl}propionate
methyl 3-{4-[9-(2-ethoxycarbonylethyl)-9H-carbazol-2-yloxy]butoxy}benzoate
{2-[4-(3-hydroxyphenoxy)butoxy]carbazol-9-yl}acetic acid
[2-(4-m-tolyloxybutoxy)carbazol-9-yl]acetic acid
{2-[4-(3-acetylaminophenoxy)butoxy]carbazol-9-yl}acetic acid
{2-[4-(3-hydroxy-5-methoxyphenoxy)butoxy]carbazol-9-yl}acetic acid
3-[4-(9-carboxymethyl-9H-carbazol-2-yloxy)butoxy]benzoic acid
3-{2-[4-(3-hydroxyphenoxy)butoxy]carbazol-9-yl}propionic acid
3-[2-(4-m-tolyloxybutoxy)carbazol-9-yl]propionic acid
3-{2-[4-(3-acetylaminophenoxy)butoxy]carbazol-9-yl}propionic acid
3-{2-[4-(3-hydroxy-5-methoxyphenoxy)butoxy]carbazol-9-yl}propionic acid
3-{4-[9-(2-carboxyethyl)-9H-carbazol-2-yloxy]butoxy}benzoic acid
{2-[4-(2-acetylphenoxy)butoxy]carbazol-9-yl}acetic acid
{2-[4-(3-acetylphenoxy)butoxy]carbazol-9-yl}acetic acid
{2-[4-(4-nitrophenoxy)butoxy]carbazol-9-yl}acetic acid
{2-[4-(3-nitrophenoxy)butoxy]carbazol-9-yl}acetic acid
{2-[4-(3-oxoindan-4-yloxy)butoxy]carbazol-9-yl}acetic acid
{2-[4-(4-chlorophenoxy)butoxy]carbazol-9-yl}acetic acid
{2-[4-(3-fluorophenoxy)butoxy]carbazol-9-yl}acetic acid
{2-[4-(3,5-dimethoxyphenoxy)butoxy]carbazol-9-yl}acetic acid
{2-[4-(2-fluorophenoxy)butoxy]carbazol-9-yl}acetic acid
{2-[4-(2-pyrrol-1-ylphenoxy)butoxy]carbazol-9-yl}acetic acid
{2-[4-(3-isopropylphenoxy)butoxy]carbazol-9-yl}acetic acid
{2-[4-(4-pyrrol-1-ylphenoxy)butoxy]carbazol-9-yl}acetic acid
{2-[4-(2-isoxazol-5-ylphenoxy)butoxy]carbazol-9-yl}acetic acid
{2-[4-(2-isoxazol-5-yl-4-methylphenoxy)butoxy]carbazol-9-yl}acetic acid
{2-[4-(2-acetylbenzofuran-7-yloxy)butoxy]carbazol-9-yl}acetic acid
{2-[4-(2-morpholin-4-ylphenoxy)butoxy]carbazol-9-yl}acetic acid
{2-[4-(6-methoxynaphthalen-2-yloxy)butoxy]carbazol-9-yl}acetic acid
{2-[4-(2-chlorophenoxy)butoxy]carbazol-9-yl}acetic acid
{2-[4-(3-chlorophenoxy)butoxy]carbazol-9-yl}acetic acid
{2-[4-(4-fluorophenoxy)butoxy]carbazol-9-yl}acetic acid
{2-[4-(4-[1,2,4]triazol-1-ylphenoxy)butoxy]carbazol-9-yl}acetic acid
(2-{4-[2-(1H-pyrazol-3-yl)phenoxy]butoxy}carbazol-9-yl)acetic acid
3-[2-(4-phenoxybutoxy)carbazol-9-yl]propionic acid
3-{2-[4-(2-acetylphenoxy)butoxy]carbazol-9-yl}propionic acid
3-{2-[4-(3-acetylphenoxy)butoxy]carbazol-9-yl}propionic acid
3-{2-[4-(4-acetylphenoxy)butoxy]carbazol-9-yl}propionic acid
3-{2-[4-(4-acetylaminophenoxy)butoxy]carbazol-9-yl}propionic acid
3-{2-[4-(4-methoxyphenoxy)butoxy]carbazol-9-yl}propionic acid
3-{2-[4-(4-nitrophenoxy)butoxy]carbazol-9-yl}propionic acid
3-{2-[4-(2-nitrophenoxy)butoxy]carbazol-9-yl}propionic acid
3-{2-[4-(3-oxoindan-4-yloxy)butoxy]carbazol-9-yl}propionic acid
3-{2-[4-(5-oxo-5,6,7,8-tetrahydronaphthalen-2-yloxy)butoxy]carbazol-9-yl}propionic acid
3-{2-[4-(1-oxoindan-5-yloxy)butoxy]carbazol-9-yl}propionic acid
3-{2-[4-(4-chlorophenoxy)butoxy]carbazol-9-yl}propionic acid
3-{2-[4-(2-methoxyphenoxy)butoxy]carbazol-9-yl}propionic acid
3-[2-(4-o-tolyloxybutoxy)carbazol-9-yl]propionic acid
3-[2-(4-p-tolyloxybutoxy)carbazol-9-yl]propionic acid
3-{2-[4-(3-methoxyphenoxy)butoxy]carbazol-9-yl}propionic acid
3-{2-[4-(naphthalen-2-yloxy)butoxy]carbazol-9-yl}propionic acid
3-{2-[4-(1-oxoindan-4-yloxy)butoxy]carbazol-9-yl}propionic acid
3-{2-[4-(5-oxo-5,6,7,8-tetrahydronaphthalen-1-yloxy)butoxy]carbazol-9-yl}propionic acid
3-{2-[4-(3-fluorophenoxy)butoxy]carbazol-9-yl}propionic acid
3-{2-[4-(3,5-dimethoxyphenoxy)butoxy]carbazol-9-yl}propionic acid
3-{2-[4-(4-imidazol-1-ylphenoxy)butoxy]carbazol-9-yl}propionic acid
3-{2-[4-(3-oxoindan-5-yloxy)butoxy]carbazol-9-yl}propionic acid
3-{2-[4-(3,4-dimethoxyphenoxy)butoxy]carbazol-9-yl}propionic acid
3-{2-[4-(2-fluorophenoxy)butoxy]carbazol-9-yl}propionic acid
3-{2-[4-(2-pyrrol-1-ylphenoxy)butoxy]carbazol-9-yl}propionic acid
3-{2-[4-(benzo[1,3]dioxol-5-yloxy)butoxy]carbazol-9-yl}propionic acid
3-(2-{4-[4-(2-oxopropyl)phenoxy]butoxy}carbazol-9-yl)propionic acid
3-{2-[4-(3-dimethylaminophenoxy)butoxy]carbazol-9-yl}propionic acid
3-{2-[4-(4-methoxymethylphenoxy)butoxy]carbazol-9-yl}propionic acid
3-{2-[4-(4-dimethylaminomethylphenoxy)butoxy]carbazol-9-yl}propionic acid
3-(2-{4-[4-(2-methoxyethyl)phenoxy]butoxy}carbazol-9-yl)propionic acid
3-{2-[4-(2,3-dimethoxyphenoxy)butoxy]carbazol-9-yl}propionic acid
3-{2-[4-(2-isoxazol-5-ylphenoxy)butoxy]carbazol-9-yl}propionic acid
3-{2-[4-(2-oxo-1,2,3,4-tetrahydroquinolin-6-yloxy)butoxy]carbazol-9-yl}propionic acid
3-{2-[4-(2-methylbenzothiazol-5-yloxy)butoxy]carbazol-9-yl}propionic acid
3-{2-[4-(2-methanesulfonylphenoxy)butoxy]carbazol-9-yl}propionic acid
3-{2-[4-(4-sulfamoylphenoxy)butoxy]carbazol-9-yl}propionic acid
3-{2-[4-(3-methoxynaphthalen-2-yloxy)butoxy]carbazol-9-yl}propionic acid
3-{2-[4-(2-isoxazol-5-yl-4-methylphenoxy)butoxy]carbazol-9-yl}propionic acid
3-{2-[4-(2-acetylbenzofuran-7-yloxy)butoxy]carbazol-9-yl}propionic acid
3-{2-[4-(1-acetylnaphthalen-2-yloxy)butoxy]carbazol-9-yl}propionic acid
3-{2-[4-(3-morpholin-4-ylphenoxy)butoxy]carbazol-9-yl}propionic acid
3-{2-[4-(6-methoxynaphthalen-2-yloxy)butoxy]carbazol-9-yl}propionic acid
3-{2-[4-(4-cyanomethylphenoxy)butoxy]carbazol-9-yl}propionic acid
3-{2-[4-(4-cyanophenoxy)butoxy]carbazol-9-yl}propionic acid
3-{2-[4-(2-cyanophenoxy)butoxy]carbazol-9-yl}propionic acid
3-{2-[4-(2-chlorophenoxy)butoxy]carbazol-9-yl}propionic acid
3-{2-[4-(3-chlorophenoxy)butoxy]carbazol-9-yl}propionic acid
3-{2-[4-(4-fluorophenoxy)butoxy]carbazol-9-yl}propionic acid
3-{2-[4-(4-[1 ,2,4]triazol-1-ylphenoxy)butoxy]carbazol-9-yl}propionic acid
3-(2-{4-[2-(1H-pyrazol-3-yl)phenoxy]butoxy}carbazol-9-yl)propionic acid
[2-(3-phenoxypropoxy)carbazol-9-yl]acetic acid
{2-[3-(2-acetylphenoxy)propoxy]carbazol-9-yl}acetic acid
{2-[3-(3-acetylphenoxy)propoxy]carbazol-9-yl}acetic acid
{2-[3-(4-acetylphenoxy)propoxy]carbazol-9-yl}acetic acid
{2-[3-(4-acetylaminophenoxy)propoxy]carbazol-9-yl}acetic acid
{2-[3-(4-methoxyphenoxy)propoxy]carbazol-9-yl}acetic acid
{2-[3-(3-nitrophenoxy)propoxy]carbazol-9-yl}acetic acid
{2-[3-(2-nitrophenoxy)propoxy]carbazol-9-yl}acetic acid
{2-[3-(3-oxoindan-4-yloxy)propoxy]carbazol-9-yl}acetic acid
{2-[3-(5-oxo-5,6,7,8-tetrahydronaphthalen-2-yloxy)propoxy]carbazol-9-yl}acetic acid
{2-[3-(1-oxoindan-5-yloxy)propoxy]carbazol-9-yl}acetic acid
{2-[3-(4-chlorophenoxy)propoxy]carbazol-9-yl}acetic acid
[2-(3-o-tolyloxypropoxy)carbazol-9-yl]acetic acid
[2-(3-m-tolyloxypropoxy)carbazol-9-yl]acetic acid
[2-(3-p-to1y1oxypropoxy)carbazo1-9-yl]acetic acid
{2-[3-(3-methoxyphenoxy)propoxy]carbazol-9-yl}acetic acid
{2-[3-(naphthalen-1-yloxy)propoxy]carbazol-9-yl}acetic acid
{2-[3-(3-acetylarninophenoxy)propoxy]carbazol-9-yl}acetic acid
{2-[3-(1-oxoindan-4-yloxy)propoxy]carbazol-9-yl}acetic acid
{2-[3-(5-oxo-5,6,7,8-tetrahydronaphthalen-1-yloxy)propoxy]carbazol-9-yl}acetic acid
{2-[3-(3-fluorophenoxy)propoxy]carbazol-9-yl }acetic acid
{2-[3-(3,5-dimethoxyphenoxy)propoxy]carbazol-9-yl}acetic acid
{2-[3-(4-imidazol-1-ylphenoxy)propoxy]carbazol-9-yl}acetic acid
{2-[3-(3-oxoindan-5-yloxy)propoxy]carbazol-9-yl}acetic acid
{2-[3-(3,4-dimethoxyphenoxy)propoxy]carbazol-9-yl}acetic acid
{2-[3-(2-acetylaminophenoxy)propoxy]carbazol-9-yl}acetic acid
{2-[3-(7-methoxynaphthalen-2-yloxy)propoxy]carbazol-9-yl}acetic acid
{2-[3-(2-fluorophenoxy)propoxy]carbazol-9-yl}acetic acid
{2-[3-(2-pyrrol-1-ylphenoxy)propoxy]carbazol-9-yl}acetic acid
{2-[3-(benzo[1,3]dioxol-5-yloxy)propoxy]carbazol-9-yl}acetic acid
(2-{3-[4-(2-oxopropyl)phenoxy]propoxy}carbazol-9-yl)acetic acid
{2-[3-(4-methoxynaphthalen-1-yloxy)propoxy]carbazol-9-yl}acetic acid
{2-[3-(2-isopropyiphenoxy)propoxy]carbazol-9-yl}acetic acid
{2-[3-(3-isopropylphenoxy)propoxy]carbazol-9-yl}acetic acid
{2-[3-(3-dimethylarninophenoxy)propoxy]carbazol-9-yl}acetic acid
{2-[3-(2-dimethylaminomethylphenoxy)propoxy]carbazol-9-yl}acetic acid
(2-{3-[4-(2-methoxyethyl)phenoxy]propoxy}carbazol-9-yl)acetic acid
{2-[3-(2,3-dimethoxyphenoxy)propoxy]carbazol-9-yl}acetic acid
{2-[3-(2-isoxazol-5-ylphenoxy)propoxy]carbazol-9-yl}acetic acid
{2-[3-(2-methylbenzothiazol-5-yloxy)propoxy]carbazol-9-yl}acetic acid
{2-[3-(2-methanesulfonylphenoxy)propoxy]carbazol-9-yl}acetic acid
{2-[3-(3-methoxynaphthalen-2-yloxy)propoxy]carbazol-9-yl}acetic acid
{2-[3-(7-methoxynaphthalen-1-yloxy)propoxy]carbazol-9-yl}acetic acid
{2-[3-(2-isoxazol-5-yl-4-methylphenoxy)propoxy]carbazol-9-yl}acetic acid
{2-[3-(2-acetylbenzofuran-7-yloxy)propoxy]carbazol-9-yl}acetic acid
{2-[3-(2-morpholin-4-ylphenoxy)propoxy]carbazol-9-yl}acetic acid
{2-[3-(2-acetylnaphthalen-1-yloxy)propoxy]carbazol-9-yl}acetic acid
{2-[3-(1-acetylnaphthalen-2-yloxy)propoxy]carbazol-9-yl}acetic acid
{2-[3-(3-morpholin-4-ylphenoxy)propoxy]carbazol-9-yl}acetic acid
{2-[3-(4-methanesulfonylphenoxy)propoxy]carbazol-9-yl}acetic acid
{2-[3-(2-cyanophenoxy)propoxy]carbazol-9-yl}acetic acid
{2-[3-(2-chlorophenoxy)propoxy]carbazol-9-yl}acetic acid
{2-[3-(3-chlorophenoxy)propoxy]carbazol-9-yl}acetic acid
{2-[3-(4-fluorophenoxy)propoxy]carbazol-9-yl}acetic acid
(2-{3-[2-(1H-pyrazol-3-yl)phenoxy]propoxy}carbazol-9-yl)acetic acid
[2-(5-phenoxypentyloxy)carbazol-9-yl]acetic acid
{2-[5-(2-acetylphenoxy)pentyloxy]carbazol-9-yl}acetic acid
{2-[5-(3-acetylphenoxy)pentyloxy]carbazol-9-yl}acetic acid
{2-[5-(4-acetylphenoxy)pentyloxy]carbazol-9-yl}acetic acid
{2-[5-(4-acetylarninophenoxy)pentyloxy]carbazol-9-yl}acetic acid
{2-[5-(4-methoxyphenoxy)pentyloxy]carbazol-9-yl}acetic acid
{2-[5-(3-cyanophenoxy)pentyloxy]carbazol-9-yl}acetic acid
{2-[5-(4-nitrophenoxy)pentyloxy]carbazol-9-yl}acetic acid
{2-[5-(3-nitrophenoxy)pentyloxy]carbazol-9-yl}acetic acid
{2-[5-(2-nitrophenoxy)pentyloxy]carbazol-9-yl}acetic acid
{2-[5-(3-oxoindan-4-yloxy)pentyloxy]carbazol-9-yl}acetic acid
{2-[5-(5-oxo-5,6,7,8-tetrahydronaphthalen-2-yloxy)pentyloxy]carbazol-9-yl}acetic acid
{2-[5-(1-oxoindan-5-yloxy)pentyloxy]carbazol-9-yl}acetic acid
{2-[5-(4-chlorophenoxy)pentyloxy]carbazol-9-yl}acetic acid
{2-[5-(2-methoxyphenoxy)pentyloxy]carbazol-9-yl}acetic acid
[2-(5-o-tolyloxypentyloxy)carbazol-9-yl]acetic acid
[2-(5-m-tolyloxypentyloxy)carbazol-9-yl]acetic acid
[2-(5-p-tolyloxypentyloxy)carbazol-9-yl]acetic acid
{2-[5-(3-methoxyphenoxy)pentyloxy]carbazol-9-yl}acetic acid
{2-[5-(naphthalen-1-yloxy)pentyloxy]carbazol-9-yl}acetic acid
{2-[5-(3-acetylaminophenoxy)pentyloxy]carbazol-9-yl}acetic acid
{2-[5-(1-oxoindan-4-yloxy)pentyloxy]carbazol-9-yl}acetic acid
{2-[5-(5-oxo-5,6,7,8-tetrahydronaphthalen-1-yloxy)pentyloxy]carbazol-9-yl}acetic acid
{2-[5-(3-fluorophenoxy)pentyloxy]carbazol-9-yl}acetic acid
{2-[5-(3,5-dimethoxyphenoxy)pentyloxy]carbazol-9-yl}acetic acid
{2-[5-(4-imidazol-1-ylphenoxy)pentyloxy]carbazol-9-yl}acetic acid
{2-[5-(3-oxoindan-5-yloxy)pentyloxy]carbazol-9-yl}acetic acid
{2-[5-(3,4-dimethoxyphenoxy)pentyloxy]carbazol-9-yl}acetic acid
{2-[5-(2-acetylaminophenoxy)pentyloxy]carbazol-9-yl}acetic acid
{2-[5-(2-fluorophenoxy)pentyloxy]carbazol-9-yl}acetic acid
{2-[5-(2-pyrrol-1-ylphenoxy)pentyloxy]carbazol-9-yl}acetic acid
{2-[5-(benzo[1,3]dioxol-5-yloxy)pentyloxy]carbazol-9-yl}acetic acid
{2-[5-(4-isopropylphenoxy)pentyloxy]carbazol-9-yl}acetic acid
{2-[5-(indan-5-yloxy)pentyloxy]carbazol-9-yl}acetic acid
{2-[5-(4-methoxynaphthalen-1-yloxy)pentyloxy]carbazol-9-yl}acetic acid
{2-[5-(2-isopropylphenoxy)pentyloxy]carbazol-9-yl}acetic acid
{2-[5-(3-isopropylphenoxy)pentyloxy]carbazol-9-yl}acetic acid
{2-[5-(3-dimethylaminophenoxy)pentyloxy]carbazol-9-yl}acetic acid
{2-[5-(2-dimethylaminomethylphenoxy)pentyloxy]carbazol-9-yl}acetic acid
(2-{5-[4-(2-methoxyethyl)phenoxy]pentyloxy}carbazol-9-yl)acetic acid
{2-[5-(2,3-dimethoxyphenoxy)pentyloxy]carbazol-9-yl}acetic acid
{2-[5-(2-isoxazol-5-ylphenoxy)pentyloxy]carbazol-9-yl}acetic acid
{2-[5-(2-oxo-1,2,3 ,4-tetrahydroquinolin-6-yloxy)pentyloxy]carbazol-9-yl }acetic acid
{2-[5-(6-cyanonaphthalen-2-yloxy)pentyloxy]carbazol-9-yl}acetic acid
{2-[5-(2-methanesulfonylphenoxy)pentyloxy]carbazol-9-yl}acetic acid
{2-[5-(2-isoxazol-5-yl-4-methylphenoxy)pentyloxy]carbazol-9-yl}acetic acid
{2-[5-(2-morpholin-4-ylphenoxy)pentyloxy]carbazol-9-yl}acetic acid
{2-[5-(2-acetylnaphthalen-1-yloxy)pentyloxy]carbazol-9-yl}acetic acid
{2-[5-(3-morpholin-4-ylphenoxy)pentyloxy]carbazol-9-yl}acetic acid
{2-[5-(6-methoxynaphthalen-2-yloxy)pentyloxy]carbazol-9-yl}acetic acid
{2-[5-(4-cyanomethylphenoxy)pentyloxy]carbazol-9-yl}acetic acid
{2-[5-(4-methanesulfonylphenoxy)pentyloxy]carbazol-9-yl}acetic acid
{2-[5-(4-cyanophenoxy)pentyloxy]carbazol-9-yl}acetic acid
{2-[5-(2-cyanophenoxy)pentyloxy]carbazol-9-yl}acetic acid
{2-[5-(2-chlorophenoxy)pentyloxy]carbazol-9-yl}acetic acid
{2-[5-(4-fluorophenoxy)pentyloxy]carbazol-9-yl}acetic acid
{2-[5-(4-[1 ,2,4]triazol-1-ylphenoxy)pentyloxy]carbazol-9-yl}acetic acid
(2-{5-[2-(1H-pyrazol-3-yl)phenoxy]pentyloxy}carbazol-9-yl)acetic acid
3-[2-(4-m-tolyloxybutoxy)carbazol-9-yl]propionic acid
3-{2-[4-(3-acetylaminophenoxy)butoxy]carbazol-9-yl}propionic acid
N-ethyl-2-[2-(4-m-tolyloxybutoxy)carbazol-9-yl]acetamide
N,N-diethyl-2-[2-(4-m-tolyloxybutoxy)carbazol-9-yl]acetamide
N-{2-[2-(4-m-tolyloxybutoxy)carbazol-9-yl]acetyl}methanesulfonamide.

5. The use of the compounds as claimed in claim 1 for producing medicaments which comprise at least one of the compounds of the formula D.

6. The medicament as claimed in claim 5 comprising suitable formulation and carrier substances.

7. The use of the medicament as claimed in claim 5, characterized in that the medicament is used for the treatment and prophylaxis of disorders.

8. The use as claimed in claim 7 for the treatment and prophylaxis of disorders connected to the EP₂ receptor.

9. The use as claimed in claim 7 for the treatment and prophylaxis of fertility disorders.

10. The use as claimed in claim 7 for the treatment and prophylaxis of menstrual complaints.

11. The use as claimed in claim 7 for the treatment and prophylaxis of endometriosis.

12. The use of the compounds as claimed in claim 1 for modulating the EP₂ receptor.

13. The use as claimed in claim 7 for the treatment and prophylaxis of pain.

14. The use as claimed in claim 7 for the treatment and prophylaxis of a disorder which is caused by elevated intraocular pressure.

15. A method for fertility control comprising administering a compound as claimed in claim 1.

16. The use as claimed in claim 7 for the treatment and prophylaxis of osteoporosis.

17. The use as claimed in claim 7 for the treatment and prophylaxis of inflammatory disorders.

18. The use as claimed in claim 17, characterized in that the inflammatory disorder is multiple sclerosis.

19. The use of the compounds of the general formula D as claimed in claim 1 in the form of a pharmaceutical preparation for enteral, parenteral, vaginal and oral administration.

20. A process for preparing the compounds as claimed in claim 1, characterized in that
a bromoalkyl side chain is inserted in a hydroxyfluorene or hydroxycarbazole derivative of type A in step 1,
an aryl ether is introduced into compounds of type B in step 2, and
an ester hydrolysis to give the end compounds D is effected in step 3.

21. An intermediate of the general formula A, B and C.
